SEQUENCE LISTING (1 sequence)

SEQ ID NO: 1 - RNA, 23 nt, Homo sapiens
agggaugguguccacuucaaauu

(19) United States
(12) Patent Application Publication
(10) Pub. No.: US 2008/0299123 A1
(43) Pub. Date: Dec. 4, 2008

(54) TREATMENT OF CHEMOTHERAPY- OR RADIOTHERAPY-RESISTANT TUMORS

(75) Inventors: Peter Altevogt, Neckardgemund (DE); Alexander Stoeck, Losheim am See (DE); Daniela Gast, Biehgheim-Bissingen (DE); Susanne Sebens Muerkoster, Kiel (DE); Heiner Schafer, Kiel (DE)

Correspondence Address: FOLEY AND LARDNER LLP;SUITE 500, 3000 K STREET NW, WASHINGTON, DC, 20007, US

(73) Assignee: Deutsches Krebsforschungszentrum Stiftung des offentlichen Rechts

(21) Appl. No.: 11/868,278

(22) Filed: Oct. 5, 2007

Related U.S. Application Data
(60) Provisional application No. 60/851,749, filed on Oct. 16, 2006.
(60) Provisional application No. 60/854,679, filed on Oct. 27, 2006.

(30) Foreign Application Priority Data
Apr. 5, 2007 (EP) PCT/EP07/03105

(51) Int. Cl. A61K 39/395; C07K 16/18; C07H 21/02; A61K 31/711; A61K 33/24; A61K 31/7105; C07H 21/04

(52) U.S. Cl. 424/137.1; 530/387.5; 536/24.5; 536/23.1; 424/649

(57) ABSTRACT

The present invention relates to the use of L1 interfering molecules, especially anti-L1 antibodies, in tumor treatment. Especially, the present invention relates to the use of said L1 interfering molecules in sensitizing tumor cells for the treatment with chemotherapeutic drugs of with radiotherapy and to the combined administration of L1 interfering molecules with chemotherapeutic drugs or with radiotherapy.

The present application claims priority of U.S. 60/851,749, filed on Oct. 16, 2007, U.S. 60/854,679, filed on Oct. 27, 2007 and of PCT/EP2007/003105, all incorporated herein by reference.

The present invention relates to the treatment of tumors and especially the treatment of tumors at least partially resistant to the treatment with chemotherapeutic drug or to radio-therapy.

The acquisition of apoptosis resistance is a hallmark of cancer progression and is frequently observed e.g. in ovarian carcinoma. The standard treatment of advanced cancer is often chemotherapy or radiotherapy. However, despite initial response to therapy, it is often observed that different carcinomas acquire resistance to chemotherapeutic drugs or radiotherapy leading to tumor recurrence and frequent death of the patients. Often, it is then decided to switch to another chemotherapeutic drug or to higher dosages. However, often no improvement of the clinical situation is observed.

L1 is a type I membrane glycoprotein of 200 to 230 kDa structurally belonging to the Ig superfamily (3, the numbering of the references corresponds to the list of example 1). L1 plays a crucial role in axon guidance and cell migration in developing nervous system (4, 5). Recent studies have also implicated L1 expression in the progression of human carcinomas. L1 expression was found on different tumors including lung cancer (6), gliomas (7), melanomas (8, 9), renal carcinoma (10, 11), colon carcinoma (12) and carcinomas of the uterine corps, cervix and urinary tract (Huszar M, Moldenhauer G, Gschwend V, Ben-Arie A, Altevogt P, Fogel M. (2006) Expression profile analysis in multiple human tumors identifies L1 (CD171) as a molecular marker for differential diagnosis and targeted therapy. Hum Pathol. 2006 August; 37(8):1000-8). Furthermore, it is known in the art that L1 is overexpressed in ovarian and endometrial carcinomas in a stage-dependent manner (13).

In the art, it has been suggested to use anti-L1 antibodies for the treatment of ovarian and endometrial tumors (cf. WO 02/04952 and WO 06/013051 and reference (35)). Furthermore, the use of anti-L1 antibodies for the treatment of breast cancer, colon cancer, cervical cancer, melanoma, neuroblastoma, small cell lung cancer, lymphoma has been suggested by Primiano et al. (WO2004037198).

Despite enormous efforts, it is still very difficult if not impossible to treat tumors resistant to chemotherapy or radiotherapy.

In a first aspect, the present invention relates to the use of an L1 interfering molecule for the preparation of a medicament for sensitizing tumor cells in a patient for the treatment with a chemotherapeutic drug or with radiotherapy.

Furthermore, the invention relates to an L1 interfering molecule for use in a method for sensitizing tumor cells in a patient for the treatment with a chemotherapeutic drug or with radiotherapy.

In the context of the present invention, it has been surprisingly found that with the help of L1 interfering molecules, e.g. siRNA or anti-L1 antibodies, it is possible to sensitize tumor cells for the treatment with a chemotherapeutic drug or with radiotherapy. Consequently, the present invention provides means for overcoming the resistance of tumor cells against these drugs.

Therefore, in a preferred embodiment of the invention, the cells to be sensitized are at least partially resistant to the treatment with said chemotherapeutic drug or to radiotherapy.

However, it is equally preferred that the cells to be sensitized are not or not yet resistant to the treatment with said chemotherapeutic drug or to radiotherapy. One consequence of said sensitization could be that the cells are rendered more susceptible to said treatment or that said resistance or partial resistance is prevented.

Without being bound to any theory, the rationale being said sensitization of the cells might be that, it has been shown in Example 1 that cells which do not express L1, or which express L1 only in a low amount before the treatment with a chemotherapeutic drug, strongly express L1 after a treatment period of only 3 weeks with a chemotherapeutic agent. Usually the administration of a chemotherapeutic for the treatment of cancer is repeated over a period of several weeks or months. Thus, L1 expression and the related resistance may at least partially be induced early during the treatment with a given chemotherapeutic or radiotherapy. Therefore, according to the invention cancer cells may be treated with an L1 interfering molecule in combination with a chemotherapeutic drug or with radiotherapy even if a resistance against said chemotherapeutic drug or with radiotherapy has not been determined before.

In the context of the present invention, the term “sensitizing” is to be understood that after the treatment with the L1 interfering molecule, the tumor cells are more susceptible to the treatment with a chemotherapeutic drug or with radiotherapy than before the treatment with an L1 interfering molecule.

Furthermore, the term “sensitizing” can be understood that due to the treatment with the L1 interfering molecule, preferably during the treatment with the L1 interfering molecule, the tumor cells are or become more susceptible to the treatment with a chemotherapeutic drug or with radiotherapy than before the treatment with an L1 interfering molecule.

This can e.g. be tested by isolating tumor cells from the patient and testing in vitro whether the treatment with an L1 interfering molecule results in a sensitization of the cells. This test can be performed as described in Example 1.

In a preferred embodiment, the cells, before the administration of the L1-interfering molecule, were not susceptible to the treatment or only susceptible to an extend that the treatment with a chemotherapeutic drug or with radiotherapy would not result in the desired therapeutic effect.

In another preferred embodiment, the tumor cells are capable of expressing L1 and are known to acquire a resistance against the respective chemotherapeutic drug or radiotherapy when treated, preferably repeatedly treated with said chemotherapeutic drug or radiotherapy.

Preferably, with the help of the L1 interfering molecule, the susceptibility is increased by at least 20%, more preferably by at least 40% and even more preferably by at least 100%, preferably as compared to cells not treated with the L1 interfering molecule.

An overview over chemotherapeutic drugs and radiotherapy is e.g. given in Remmington's Pharmaceutical Sciences, 5^{th} ed., chapter 33, in particular pages 624 to 652.

Any of numerous chemotherapeutic drugs can be used in the methods and uses of the invention. These compounds fall into several different categories, including, for example, alkylating agents, antineoplastic antibiotics, antimetabolites, and natural source derivatives.

Examples of alkylating agents that can be used in the invention include busulfan, carboplatin, carmustine, chlorambucil, cisplatin, oxaliplatin, cyclophosphamide (i.e., cytoxan), dacarbazine, ifosfamide, lomustine, mecholarethamine, melphalan, procarbazine, streptozocin, and thiotepa.

Examples of antineoplastic antibiotics include bleomycin, dactinomycin, daunorubicin, doxorubicin, idarubicin, mitomycin (e.g., mitomycin C), mitoxantrone, pentostatin, and plicamycin.

Examples of antimetabolites include fluorodeoxyuridine, cladribine, cytarabine, floxuridine, fludarabine, fluorouracil (e.g., 5-fluorouracil (5FU)), gemcitabine, hydroxyurea, mercaptopurine, methotrexate, thioguanine, folic acid derivatives (e.g. 5-formyl tetrahydrofolic acid), and capecitabine.

Examples of natural source derivatives include docetaxel, etoposide, irinotecan, taxanes (e.g. paclitaxel or docetaxel), teniposide, topotecan, vinblastine, vincristine, vinorelbine, prednisone, and tamoxifen.

Additional examples of chemotherapeutic agents that can be used in the invention include asparaginase and mitotane.

Furthermore, also C2 ceramide can be used.

In an especially preferred embodiment, the chemotherapeutic drug is selected from the group consisting of actinomycin-D, mitomycin C, cisplatin, doxorubicin, etoposide, gemcitabine, verapamil, podophyllotoxin, 5-FU, taxans such as paclitaxel, carboplatin, cyclophosphamide, vinorelbine, oxaliplatin, capecitabine, doxorubicin, and ifosfamide.

According to a further preferred embodiment of the invention, the term “chemotherapeutic drug” also includes an antibody or fragment thereof being capable of inducing apoptosis in the cell. Examples of such antibodies include antibodies binding to tyrosin kinases, e.g. the EGF receptor.

According to the invention, the term “radiotherapy” refers to each radiation therapy which is commonly used to treat tumors cells. In a preferred embodiment, this therapy include γ-rays, X-rays, microwaves, UV radiation as well as the direct delivery of radio-isotopes to or next to tumor cells (brachytherapy).

As mentioned above, the object of this aspect of the invention is to sensitize tumor cells for the treatment with a chemotherapeutic drug or with radiotherapy. Consequently, in a preferred embodiment, after or during the sensitization with the L1 interfering molecule, the patient is further treated with said chemotherapeutic drug or with said radiotherapy. The regimen for treatment with a chemotherapeutic drug or with radiotherapy is known in the art.

According to the invention, the term “L1 interfering molecule” may relate to a molecule which binds to L1 (i.e. an L1 binding molecule). In this context, the L1 interfering molecule binding to L1 can bind to L1 extracellularly (e.g. an antibody or an anticalin) or intracellularly (e.g. a low molecular weight molecule).

Methods for determining whether a given molecule binds to L1 are known in the art and include e.g. ELISA, Western-Blotting, immunohistochemistry and FACS staining.

Furthermore, the term “L1 interfering molecule” may relate to a nucleic acid in the tumor cell encoding or being complementary to L1 coding sequences, e.g. L1 encoding DNA or mRNA or parts thereof and when entering a tumor cell modulates, preferably inhibits L1 expression in the tumor cell. Such molecules are discussed below with reference to siRNA, antisense molecules and ribozymes.

According to the invention, such inhibition may be completely or partially, e.g. the expression may be reduced by at least 50% or by at least 80%.

Furthermore, this term also relates to molecules which act downstream in the activity cascade of L1. This includes e.g. molecules binding to protein kinases activated upon binding of a ligand to L1.

According to this aspect of the invention, the L1 interfering molecule is used to sensitize tumor cells to the treatment with a chemotherapeutic drug or with radiotherapy. Examples 1 and 3 provide an experimental test system for testing whether a given L1 interfering molecule is capable of sensitizing tumor cells. Example 1 demonstrates that siRNA directed against L1 is able to abolish chemoresistance in cell culture, while Example 3 demonstrates the same fact for anti-L1 antibodies.

Consequently, in a preferred embodiment, an L1 interfering molecule according to this aspect of the invention is a molecule as defined above which is capable of sensitizing tumor cells for the treatment with a chemotherapeutic drug or with radiotherapy.

Furthermore, it would be possible to evaluate whether a given molecule is capable of sensitizing tumor cells by performing appropriate clinical studies and determining whether the given compound has a statistical significant effect.

Preferably, said L1 interfering molecule is selected from the group consisting of anti-L1 antibodies, antibody fragments thereof, siRNA, antisense RNA or DNA, ribozymes, low molecular weight molecules, soluble L1, L1-binding scaffolds such as anticalins, and L1 ligands or parts thereof.

In an especially preferred embodiment, the L1 interfering molecule is an anti-L1 antibody or an antibody fragment thereof.

In the context of the present invention it has been found that anti-L1 antibodies and siRNA can be used for sensitizing tumor cells. The experiments provided in Example 3 demonstrate that anti L1 antibodies are able to abolish chemoresistance in cell culture. Furthermore, the experiments provided in Example 4 demonstrate that pretreatment of cultured cells with anti-L1 antibodies leads to a sensibilization towards apoptosis induced by chemotherapeutics.

Without being bound to any theory, it is believed that the mode of action of antibodies is different to that of siRNA. Especially, siRNA acts by blocking expression of the L1 molecule, while for the activity of anti-L1 antibodies, it is important that the L1 molecule itself is present. Furthermore, as shown in example 2, anti-L1 antibodies apparently mediate a signal through the L1 molecule, because binding of anti-L1 antibodies results in a change in the expression of genes related to apoptosis. Therefore, since in the context of the present invention, it has been found that although anti-L1 antibodies and siRNA have different modes of action, both agents are capable of sensitizing tumor cells to the treatment with a chemotherapeutic drug, these findings allow a generalization to all L1 interfering molecules.

According to the present invention the term antibody or antibody fragment is understood as meaning antibodies (e.g. polyclonal or monoclonal antibodies as well as recombinantly produced antibodies) or antigen-binding parts thereof, which may have been prepared by immortalizing B-cells and/or recombinantly and, where appropriate, modified, such as chimeric antibodies, humanized antibodies, multifunctional antibodies, bispecific or oligospecific antibodies, single-stranded antibodies and F(ab) or F(ab)₂ fragments (see, for example, EP-B1-0 368 684, U.S. Pat. No. 4,816,567, U.S. Pat. No. 4,816,397, WO 88/01649, WO 93/06213 or WO 98/24884), preferably produced with the help of a FAB expression library.

According to the invention, the antibody or antibody fragment binds to the extracellular portion of L1.

Preferably, according to the invention, monoclonal antibodies are used, although it is equally envisaged to use polyclonal antibodies. These antibodies can be produced according to standard methods as described above and are also commercially available from e.g. Santa Cruz Biotechnology, R&D Systems, Abeam or Signet.

Methods for the preparation of antibodies and antibody fragments are well known in the art and are e.g. described in Antibodies—a Laboratory manual, E. Harlow et al, Cold Spring Harbor Laboratory Press, 1998.

As an alternative to the classical antibodies it is also possible, for example, to use protein scaffolds against L1, e.g. anticalins which are based on lipocalin (Beste et al. (1999) Proc. Natl. Acad. Sci. USA, 96, 1898-1903). The natural ligand-binding sites of the lipocalins, for example the retinol-binding protein or the bilin-binding protein, can be altered, for example by means of a “combinatorial protein design” approach, in such a way that they bind to selected haptens, here to L1 (Skerra, 2000, Biochim. Biophys. Acta, 1482, 337-50). Other known protein scaffolds are known as being alternatives to antibodies for molecular recognition (Skerra (2000) J. Mol. Recognit., 13, 167-187).

The procedure for preparing an antibody or antibody fragment is effected in accordance with methods which are well known to the skilled person, e.g. by immunizing a mammal, for example a rabbit, with L1 or fragments thereof, where appropriate in the presence of, for example, Freund's adjuvant and/or aluminium hydroxide gels (see, for example, Diamond, B. A. et al. (1981) The New England Journal of Medicine: 1344-1349). The polyclonal antibodies which are formed in the animal as a result of an immunological reaction can subsequently be isolated from the blood using well known methods and, for example, purified by means of column chromatography. Monoclonal antibodies can, for example, be prepared in accordance with the known method of Winter & Milstein (Winter, G. & Milstein, C. (1991) Nature, 349, 293-299). An alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089.) Such chimeric and humanized monoclonal antibodies can be produced by recombinant DNA techniques known in the art.

Antibody fragments that contain the idiotypes of the protein can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments.

In a preferred embodiment of the invention, the L1 interfering molecule, preferable an anti-L1 antibody, binds both soluble and membrane-bound L1. In a preferred embodiment, the L1 interfering molecule is capable of preventing soluble L1 from binding to cell surface receptors including integrins or L1. Assays for determining whether a given molecule has this capacity are known in the art and include functional assays measuring a reduction of motility or of invasive capacity.

The production and use of siRNAs as tools for RNA interference in the process to down regulate or to switch off gene expression, here L1 gene expression, is e.g. described in Elbashir, S. M. et al. (2001) Genes Dev., 15, 188 or Elbashir, S. M. et al. (2001) Nature, 411, 494. Preferably, siRNAs exhibit a length of less than 30 nucleotides, wherein the identity stretch of the sense strang of the siRNA is preferably at least 19 nucleotides.

An “antisense” nucleic acid as used herein refers to a nucleic acid capable of hybridizing to a sequence-specific portion of a component protein RNA (preferably mRNA) by virtue of some sequence complementarity. The antisense nucleic acid may be complementary to a coding and/or noncoding region of a component protein mRNA.

The antisense nucleic acids are of at least six nucleotides and are preferably oligonucleotides, ranging from 6 to about 200 nucleotides. In specific aspects, the oligonucleotide is at least 10 nucleotides, at least 15 nucleotides, at least 100 nucleotides, or at least 200 nucleotides.

Ribozymes are also suitable tools to inhibit the translation of nucleic acids, here the Eph receptor gene, because they are able to specifically bind and cut the mRNAs. They are e.g. described in Amarzguioui et al. (1998) Cell. Mol. Life. Sci., 54, 1175-202; Vaish et al. (1998) Nucleic Acids Res., 26, 5237-42; Persidis (1997) Nat. Biotechnol., 15, 921-2 or Couture and Stinchcomb (1996) Trends Genet., 12, 510-5.

LMW molecules (low molecular weight molecules) are molecules which are not proteins, peptides, antibodies or nucleic acids, and which exhibit a molecular weight of less than 5000 Da, preferably less than 2000 Da, more preferably less than 1000 Da, most preferably less than 500 Da. Such LMWs may be identified in High-Through-Put procedures starting from libraries.

In the context of the present invention, it is envisaged to sensitize tumor cells of any cell type. Preferably, the tumor cells are of a type selected from the group consisting of astrocytoma, oligodendroglioma, meningioma, neurofibroma, glioblastoma, ependymoma, Schwannoma, neurofibrosarcoma, medulloblastoma, melanoma cells (e.g. malignant melanoma), pancreatic cancer cells, prostate carcinoma cells, head and neck cancer cells, breast cancer cells, lung cancer cells (e.g. small cancer, non-small cancer), colon cancer cells (e.g. adenocarcinoma of the colon), colorectal cancer cells, gastrointestinal stromal tumor cells, ovarian cancer cells, endometrial cancer cells, renal cancer cells, neuroblastomas, squamous cell carcinomas, medulloblastomas, hepatoma cells and mesothelioma, epidermoid carcinoma, clear cell adenocarcinoma cells and serous adenocarcinoma of the uterine corps cells, cervix carcinoma cells, urinary tract adenocarcinoma cells, Pheochromocytoma cells, neuroma cells, neurillemoma cells, and paranganglioma cells.

Furthermore, it is preferred that the tumor cells are epithelial tumor cells, preferably ovarian cancer cells, endometrial cancer cells, adenocarcinoma of the colon, pancreatic carcinoma cells or small cell lung cancer cells.

In another preferred embodiment the tumor cells are melanoma cells.

Throughout the invention, if “tumor cells” are addressed, this means either the plural (“tumor cells”) or singular (“tumor cell”). However, even if it is contemplated within the present invention that only one tumor cell is treated or sensitized, the skilled person will appreciate that in most case more than one tumor cell (i.e. tumor cells) is treated or sensitized.

As discussed above, the L1 interfering molecules are used for the preparation of a pharmaceutical composition or medicament. Throughout the invention, the terms “pharmaceutical composition” and “medicament” are used interchangeable.

In general, the pharmaceutical compositions of the present invention comprise a therapeutically effective amount of a therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the therapeutic of the invention, which will be effective in the treatment of a particular disorder or condition, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. In general, suppositories may contain active ingredient in the range of 0.5% to 10% by weight; oral formulations preferably contain 10% to 95% active ingredient.

Various delivery systems are known and can be used to administer a therapeutic of the invention, e.g., encapsulation in liposomes, microparticles, and microcapsules: use of recombinant cells capable of expressing the therapeutic, use of receptor-mediated endocytosis (e.g., Wu and Wu, 1987, J. Biol. Chem. 262:4429-4432); construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the therapeutic can be delivered in a vesicle, in particular a liposome (Langer, 1990, Science 249:1527-1533), more particular a cationic liposome (WO 98/40052).

In yet another embodiment, the therapeutic can be delivered via a controlled release system. In one embodiment, a pump may be used (Langer, supra). In yet another embodiment, a controlled release system can be placed in proximity of the therapeutic target, thus requiring only a fraction of the systemic dose.

Within the context of this aspect of the invention, the invention also includes a method for sensitizing tumor cells in a patient for the treatment with a chemotherapeutic drug or with radiotherapy, comprising administering to the patient an efficient amount of an L1 interfering molecule. All embodiments described above also apply to this method of the invention.

Throughout the invention, the term “effective amount” means that a given molecule or compound is administered in an amount sufficient to obtain a desired therapeutic effect. In case that, throughout the invention, two compounds are administered in a therapeutic effective amount, this includes that one or each of the compounds may be administered in a subtherapeutic amount, i.e. that the amount of each compound on its own is not sufficient to provide a therapeutic effect, but that the combination of the compounds results in the desired therapeutic effect. However, it is also included within the present invention that each of the compounds on its own is administered in a therapeutically effective amount.

In a second aspect of the invention, the invention relates to the use of an L1 interfering molecule for the preparation of a medicament for the treatment of tumor cells in a patient previously treated with a chemotherapeutic drug or with radiotherapy.

Furthermore, the present invention relates to an L1 interfering molecule for use in a method for the treatment of tumor cells in a patient previously treated with a chemotherapeutic drug or with radiotherapy.

In the context of this aspect of the invention, it has been surprisingly found that tumor cells treated with a chemotherapeutic drug or with radiotherapy exhibit an increased L1 expression in comparison to not treated tumor cells. This finding leads to a novel indication for L1 interfering molecules in the context of tumor therapy, namely to the treatment of tumor cells in patients previously treated with a chemotherapeutic drug or with radiotherapy because in these patients the L1 interfering molecule should be especially efficient.

The treatment of tumor cells with L1 interfering molecules has already been described in WO 02/04952 and WO 06/013051, incorporated herein by reference.

In the context of the present invention, the term “previously treated” may include patients which have already been treated with a chemotherapeutic drug or with radiotherapy in the course of a separated regimen which has taken place e.g. within the last six or eight months. It also includes patients that already have been treated with the respective chemotherapeutic drug or with radiotherapy in a way that the tumor cells have been become at least partially resistant to said treatment.

In the course of tumor treatment with chemotherapeutic drugs or radiotherapy it is in most to cases observed that after an initial response of the tumor to such therapy (tumor mass reduction or stabilization of the disease) the tumors start to progress again. Such progression usually starts upon weeks or months after such therapy. Typically these tumors are then resistant to further treatment with the previously applied chemotherapeutic drug and other treatment modalities are wanted. As described above it has been found that such resistant tumors express L1 and therefore become a target for L1 interfering molecules.

Therefore, according to this embodiment of the invention, the term “previously treated” preferably means that the patient previously received such treatment, such treatment showed an initial effect and at the time of therapy with the L1 interfering molecule the tumor is progressing again.

Furthermore, the term “previously treated” may also be seen in a context where the L1 interfering molecule and the chemotherapeutic drug or radiotherapy are used within the same regimen, meaning that the treatments are given within one treatment schedule. In this context “in one treatment schedule” means that the treatment are applied at the same time, one after another or intermittently, but—in contrast to above—time distances between the individual treatments are short (within one week or within 2-4 days) and, if a treatment success is seen, one does not wait for tumor progression before the next treatment is applied.

Preferably, in this context, the invention includes the case where a patient is treated with a chemotherapeutic drug or with radiotherapy and subsequently, preferably within one week or less and more preferably within 2-4 days, a treatment with an L1 interfering molecule is started. In a further preferred embodiment several cycles of chemotherapy or radiotherapy on one side and treatment with an L1 interfering molecule are made, with intervals of preferably one week or less and more preferably within 2-4 days.

In a preferred embodiment, the patient is at least partially resistant to the treatment with said chemotherapeutic drug or with radiotherapy, an effect often observed in the course of said treatment types (see above).

In a further aspect, the invention relates to the use of an L1 interfering molecule for the preparation of a medicament for the treatment of tumor cells in a patient at least partially resistant to treatment with a given chemotherapeutic drug or with radiotherapy.

Furthermore, the present invention relates to an L1 interfering molecule for use in a method for the treatment of tumor cells in a patient at least partially resistant to the treatment with a given chemotherapeutic drug or with a given chemotherapeutic drug or with radiotherapy.

In the context of the present invention, the term “resistant to treatment” means that the respective tumor cell does not react to the treatment with a chemotherapeutic drug or with radiotherapy in a complete manner. This means preferably that rather, with respect to this tumor cell, treatment with said chemotherapeutic drug or radiotherapy is rather ineffective or even shows no effects. According to the invention, the term “partially” means that the respective effect is not complete.

In a further aspect of the invention, the invention relates to the use of an L1 interfering molecule for the preparation of a medicament for the treatment of tumor cells in a patient, wherein the L1 interfering molecule is administered in combination with a chemotherapeutic drug or with radiotherapy, preferably wherein the chemotherapeutic drug or the radiotherapy is administered prior to the L1 interfering molecule.

Furthermore, the present invention relates to an L1 interfering molecule for use in a method for the treatment of tumor cells in a patient, wherein the L1 interfering molecule is administered in combination with a chemotherapeutic drug or with radiotherapy, preferably wherein the chemotherapeutic drug or the radiotherapy is administered prior to the L1 interfering molecule.

According to the invention, the term “treatment of tumor cells” includes both the killing of tumor cells, the reduction of the proliferation of tumor cells (e.g. by at least 30%, at least 50% or at least 90%) as well as the complete inhibition of the proliferation of tumor cells. Therefore, this term also relates to the treatment of the respective tumorigenic disease, especially to the treatment of a solid tumor formed by said tumor cells or to the treatment of tumorigenic diseases Furthermore, this term includes the prevention of a tumorigenic disease, e.g. by killing of cells that may or are prone to become a tumor cell in the future

According to the invention, the term “in combination with” includes any combined administration of the L1 interfering molecule and the chemotherapeutic drug or radiotherapy. This may include the simultaneous application of the drugs or radiotherapy or, preferably, a separate administration. In case that a separate administration is envisaged, one would preferably ensure that a significant period of time would not expire between the time of delivery, such that the L1 interfering molecule and the chemotherapeutic drug or radiotherapy would still be able to exert an advantageously combined effect on the cell. In such instances, it is preferred that one would contact the cell with both agents within about one week, preferably within about 4 days, more preferably within about 12-36 hours of each other.

The rational behind this aspect of the invention is that the administration of chemo-therapeutic drugs or the treatment with radiotherapy may lead to an increase of L1 expression on the surface of the tumor cells which in turn makes the tumor cells a better target for the L1 interfering molecule. Furthermore, it is shown in example 3 and 4 of the application that the treatment with an L1 interfering molecule (eg. siRNA or an anti-L1 antibody) increases apoptosis in cancer cells treated with a chemotherapeutic agent.

Therefore, this aspect of the invention also encompasses treatment regimens where an L1 interfering molecule is administered in combination with the chemotherapeutic drug or radiotherapy in various treatment cycles wherein each cycle may be separated by a period of time without treatment which may last e.g. for two weeks and wherein each cycle may involve the repeated administration of the L1 interfering molecule and/or the chemotherapeutic drug or radiotherapy. For example such treatment cycle may encompass the treatment with a chemotherapeutic drug or with radiotherapy, followed by e.g. the twice application of the L1 interfering molecule within 2 days.

Throughout the invention, the skilled person will understand that the individual therapy to be applied will depend on the e.g. physical conditions of the patient or on the severity of the disease and will therefore have to be adjusted on a case to case basis.

Especially in the course of such repeated treatment cycles, it is also envisaged within the present invention that the L1 interfering molecule is administered prior to the chemotherapeutic drug or the radiotherapy.

In these aspects of the invention, the L1 interfering molecule is used to treat tumor cells. The publication Arlt et al. (number (35) in the reference list to Example 1) as well as Example 2 demonstrate an assay for the killing of tumor cells with an L1 interfering molecule, here an anti-L1 antibody. Consequently, in a preferred embodiment, according to this aspect of the invention, an L1 interfering molecule is a molecule as defined above which is capable of treating tumor cells.

For the above aspects of the invention, preferably the definition of the L1 interfering molecule is as explained above. Preferably, the L1 interfering molecule is selected from the group consisting of anti-L1 antibodies, siRNA, antisense RNA or DNA, ribozymes, low molecular weight molecules, soluble L1, anticalins, and L1 ligands.

Especially with respect to the anti-L1 antibodies, the same applies as discussed above.

In a further preferred embodiment, the L1 interfering molecule is further linked to a toxin, with the consequence that upon binding of the anti-L1 antibody to L1, the toxin exerts its effects on the tumor cell with the result that the tumor cell is treated. Preferably, as already mentioned above, the L1 interfering molecule is a L1 binding molecule, more preferably an anti-L1 antibody.

According to the present invention, the term “treatment” refers to all sorts of treatment of tumor cells including killing the tumor cells or stopping the growth of tumor cells. Furthermore, the term also includes the prevention of tumor formation, especially of formation of metastases.

With respect to the treatment of tumor cells, the tumor cells might be of the same type as explained above, namely of a type selected from the group consisting of astrocytoma, oligodendroglioma, meningioma, neurofibroma, glioblastoma, ependymoma, Schwannoma, neurofibrosarcoma, medulloblastoma, melanoma cells (e.g. malignant melanoma), pancreatic cancer cells, prostate carcinoma cells, head and neck cancer cells, breast cancer cells, lung cancer cells (e.g. small cancer, non-small cancer), colon cancer cells (e.g. adenocarcinoma of the colon), colorectal cancer cells, gastrointestinal stromal tumor cells, ovarian cancer cells, endometrial cancer cells, renal cancer cells, neuroblastomas, squamous cell carcinomas, medulloblastomas, hepatoma cells and mesothelioma, epidermoid carcinoma, clear cell adenocarcinoma cells and serous adenocarcinoma of the uterine corps cells, cervix carcinoma cells, urinary tract adenocarcinoma cells, Pheochromocytoma cells, neuroma cells, neurillemoma cells, and paranganglioma cells.

Furthermore, it is preferred that the tumor cells are epithelial tumor cells, preferably ovarian cancer cells, endometrial cancer cells, adenocarcinoma of the colon, pancreatic carcinoma cells or small cell lung cancer cells.

In another preferred embodiment the tumor cells are melanoma cells.

In the context of the above aspects of the invention, the invention also relates to a method for treating tumor cells in a patient previously treated with a chemotherapeutic drug or with radiotherapy, comprising administering to the patient a therapeutically effective amount of an L1 interfering molecule. Furthermore, the invention relates to a method for treating tumor cells in a patient at least partially resistant to treatment with a given chemotherapeutic drug or with radiotherapy, comprising administering to the patient a therapeutically effective amount of an L1 interfering molecule. Furthermore, the invention relates to a method for treating tumor cells in a patient, comprising administering to the patient a therapeutically effective amount of an L1 interfering molecule in combination with a chemotherapeutic drug or with radiotherapy.

With respect to these methods of the invention, all embodiments described above for the other uses or methods of the invention also apply.

With respect to the relation between L1 and apoptosis, is has already been mentioned above that Examples 3 and 4 demonstrate that the treatment with an L1 interfering molecule promotes apoptosis in cancer cells induced by chemotherapeutic agents. Within the context of the current application “promote apoptosis” means to increase cellular events that are related to apoptotic cell death (e.g. increase caspase-3/-7 activity in the cell). Furthermore, Example 1 demonstrates that the induction of apoptosis by chemotherapeutic agents is inhibited in cells expressing L1 in comparison to non-L1 expressing cells.

Consequently, in a preferred embodiment of the uses and methods of the invention, the L1 interfering molecule promotes apoptosis in said tumor cell or cells, preferably in tumor cells which have been treated, are treated or are to be treated with a chemotherapeutic drug.

Thus, in another aspect, the present invention also relates to a method of promoting chemotherapeutic drug or radiotherapy induced apoptosis in a eukaryotic cancer cell by treating said cell with an L1 interfering molecule. Furthermore, the present invention also relates to a method of promoting chemotherapeutic drug induced apoptosis in the tumor cells of a patient by administering an L1 interfering molecule to said patient. In a preferred embodiment, apoptosis is induced by a chemotherapeutic drug.

With respect to these methods of the invention, all embodiments described above for the other uses or methods of the invention also apply.

The invention is further illustrated by the following figures and examples which are not intended to limit the scope of the invention.

### SHORT LEGENDS TO THE FIGURES

FIG. 1. Role of L1 in apoptosis resistance studied in HEK 293 and CHO cells

(A) FACS analysis of HEK293 and HEK293-hL1 cells. Cells were analysed by cytofluorographic analysis using mAb L1-11A to L1 followed by PE-conjugated anti-mouse IgG antibody (B and C). Induction of apoptosis by the indicated compounds and Nicoletti staining. The percentage in region Ml of the histogram indicates the percentage of living cells that is graphically depicted in (C). (D) FACS analysis of CHO and CHO-hL1 cells. Cells were analysed as described in (A). (E and F) Induction of apoptosis by the indicated compounds and the indicated length of time. The rate of apoptosis was determined by Nicoletti staining and the percentage of living cells after treatment is depicted.

FIG. 2. Analysis of L1 dependent signaling in HEK 293 cells

(A) Phosphorylation of ERK1/2, FAK, and PAK 1 was analyzed in HEK293 and HEK293-hL1 grown in serum. Relative band intensities as revealed by densitometric scanning are shown. (B) Analysis of Bcl-2 expression. Relative band intensities were determined by densitometric scanning and were normalized using the β-actin loading control and are graphically depicted.

FIG. 3. Soluble L1 can partially rescue HEK293 cells from apoptosis

(A) HEK293 and HEK293-hL1 cells were treated with staurosporine for the indicated length of time in the presence or absence of purified soluble L1 (10 μg/ml). Cell survival was determined by Nicoletti staining. (B) Analysis of FAK phosphorylation in HEK293 cells after the addition of soluble L1.

FIG. 4. Effect of L1-depletion on apoptosis resistance in OVMz ovarian carcinoma cells

(A) OVMz cells were transfected with L1-specific siRNA or control siRNA. After 48 hrs, cells were stained with mAb L1-11A to L1 followed by PE-conjugated anti-mouse IgG antibody and subjected to FACS analysis. (B) Cell lysates were analyzed by Western blot analysis using antibodies to the L1 ectodomain (mAb L1-11A) or the cytoplasmic portion (pcytL1). The L1-32 fragment is the ADAM10-mediated ectodomain cleavage fragment [14].

(C) Phosphorylation of ERK1/2, FAK, and PAK 1 was analyzed in L1-siRNA depleted cell. (D and E) analysis of apoptosis by Nicoletti staining.

FIG. 5. Cisplatin treatment augments L1 expression in m130 cells

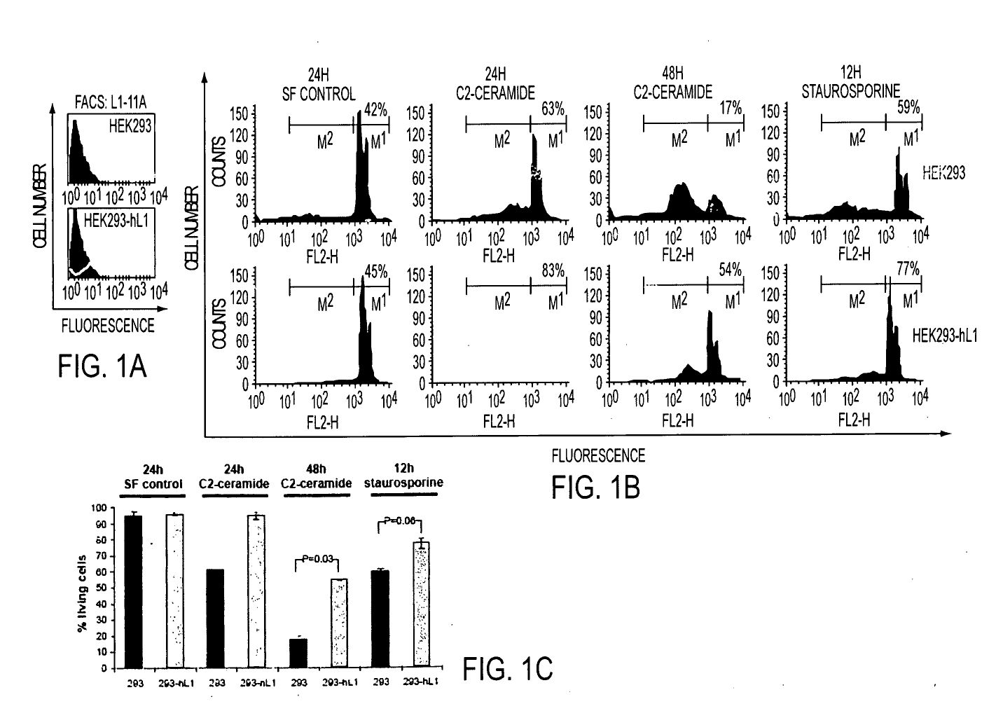

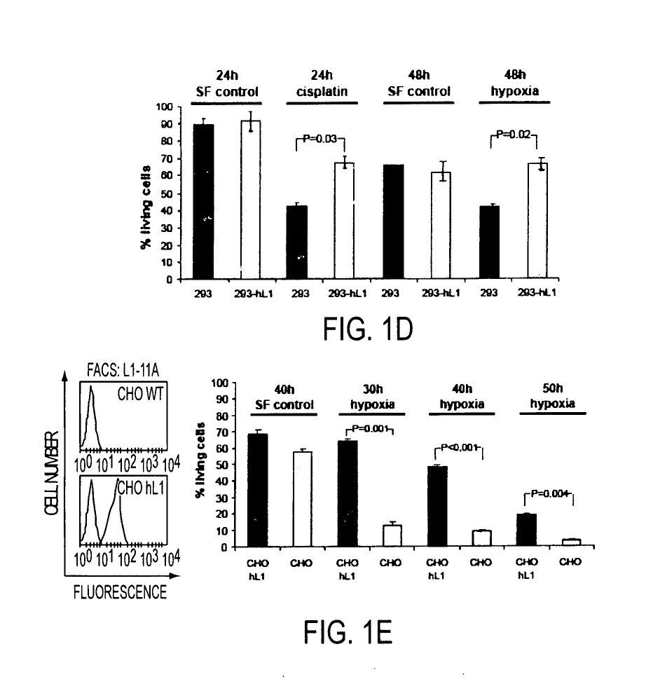

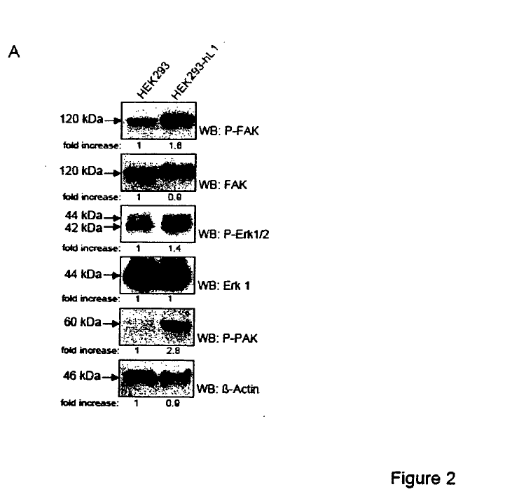

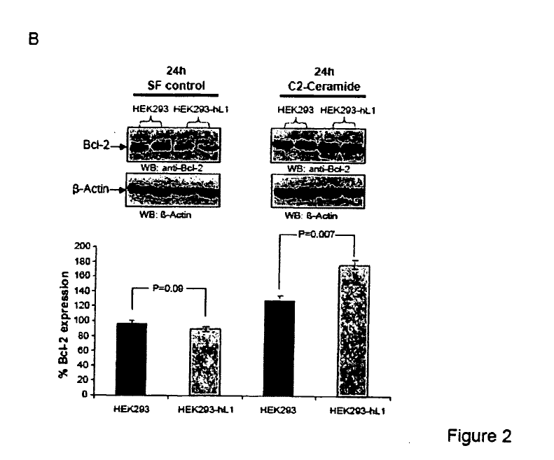

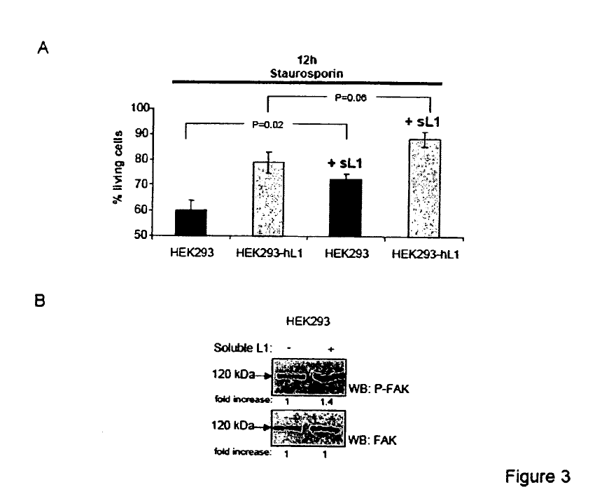

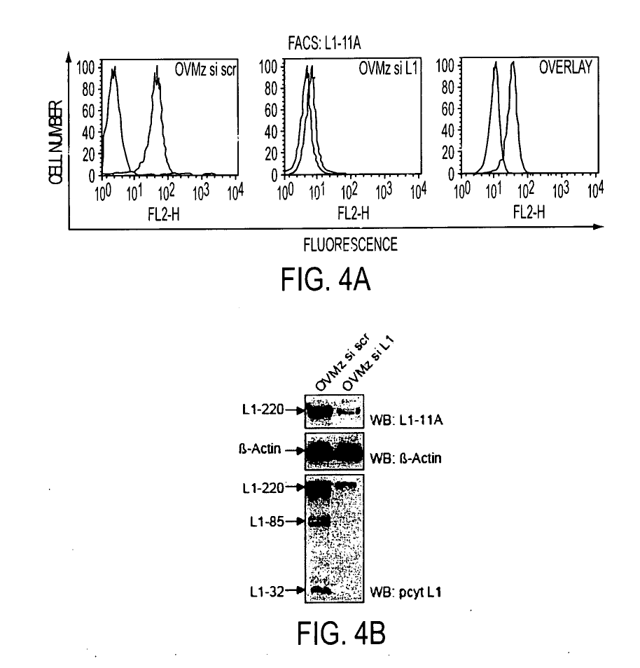

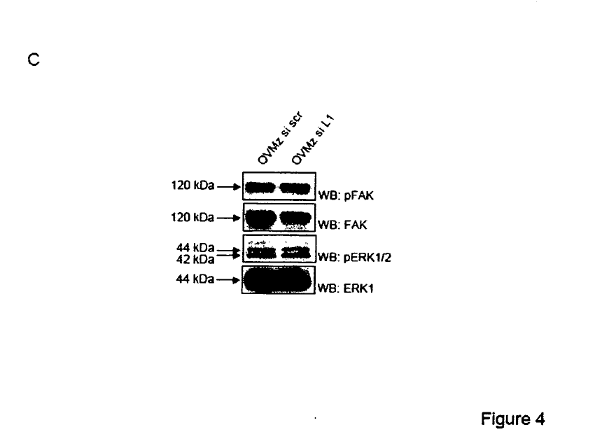

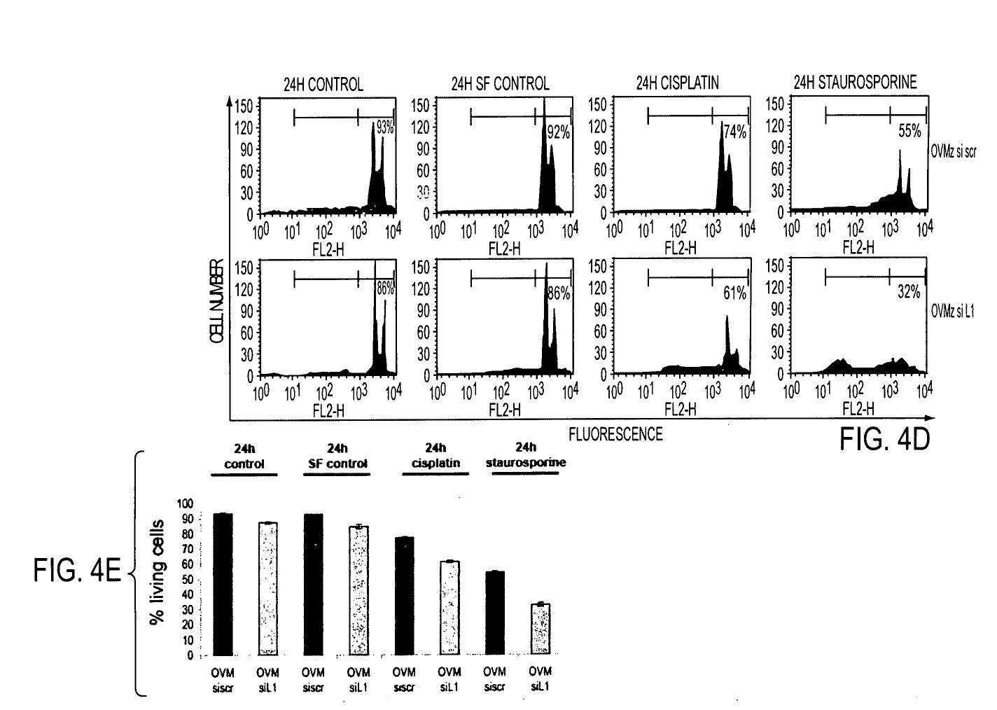

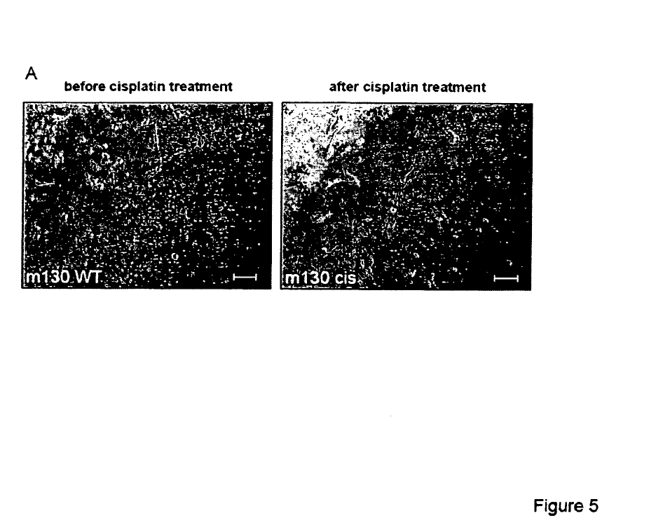

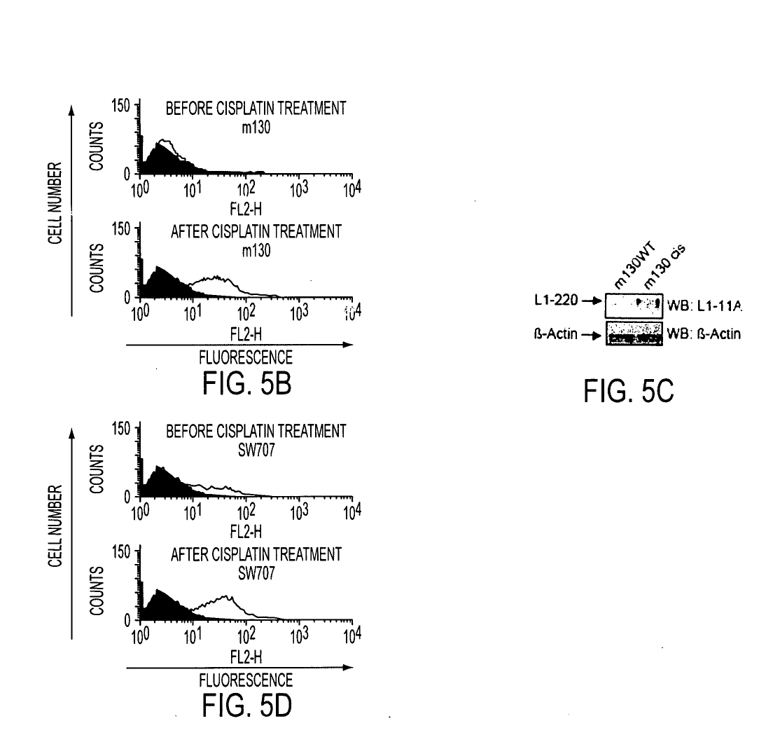

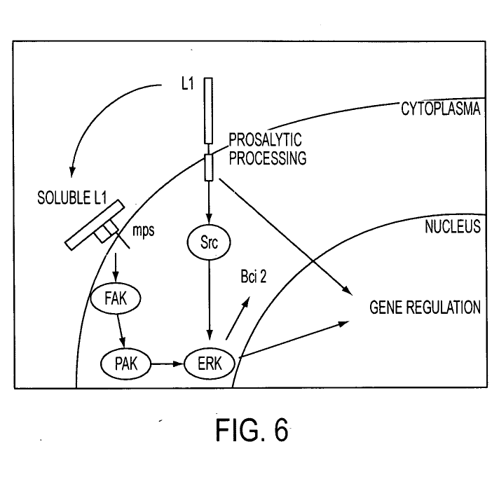

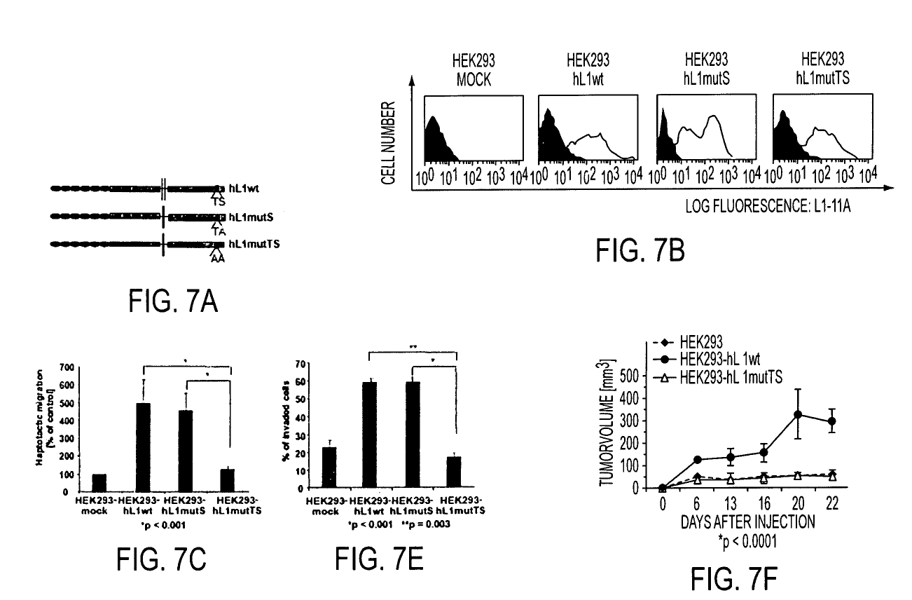

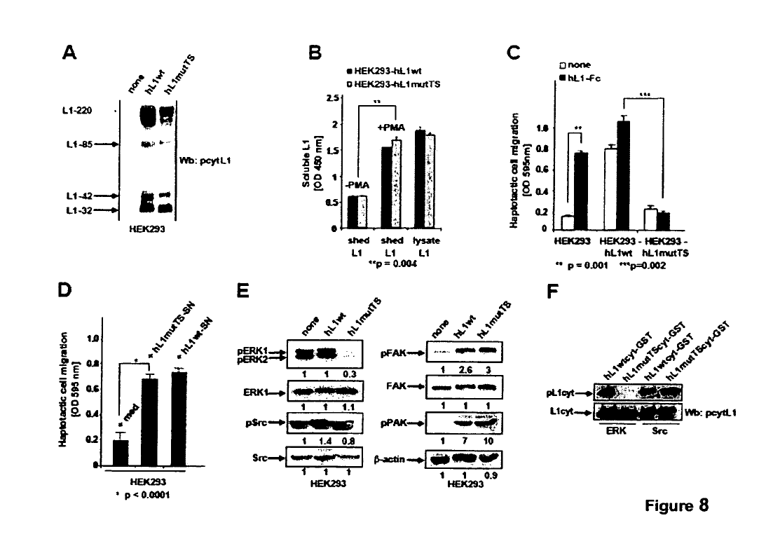

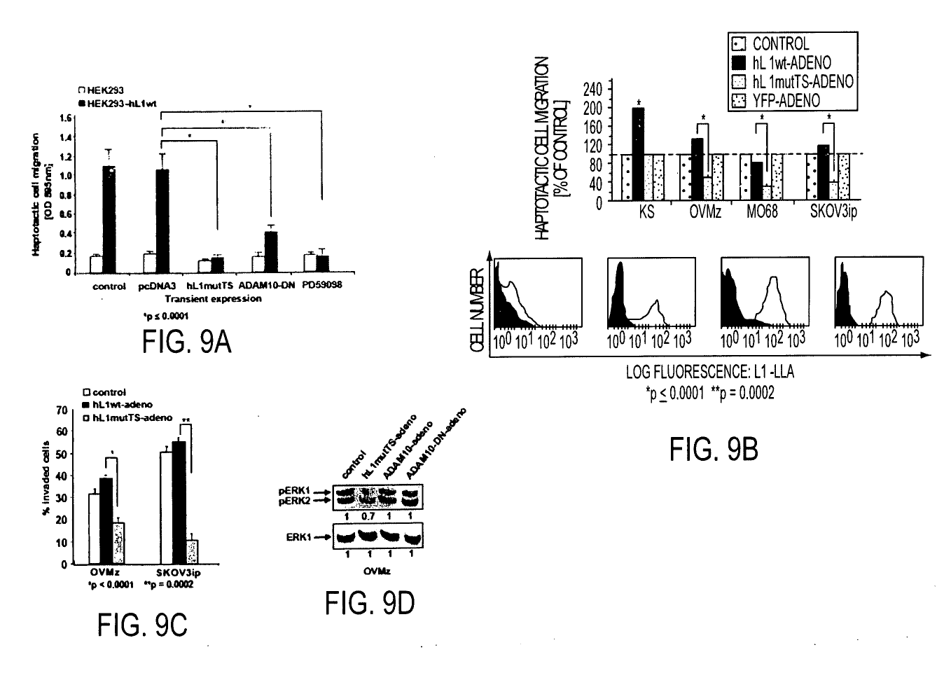

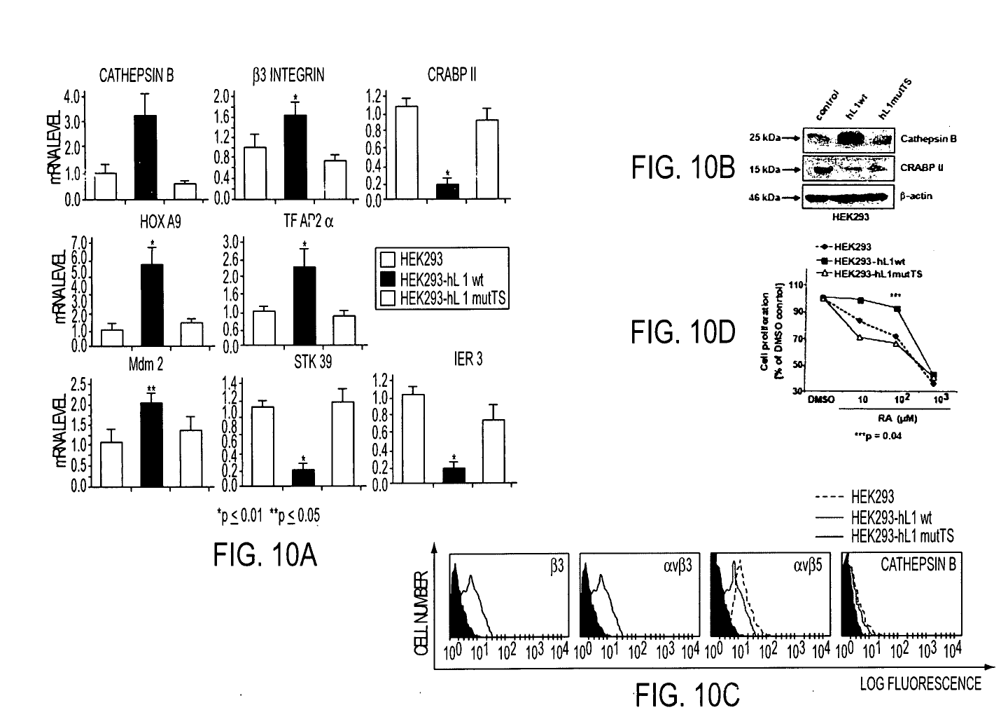

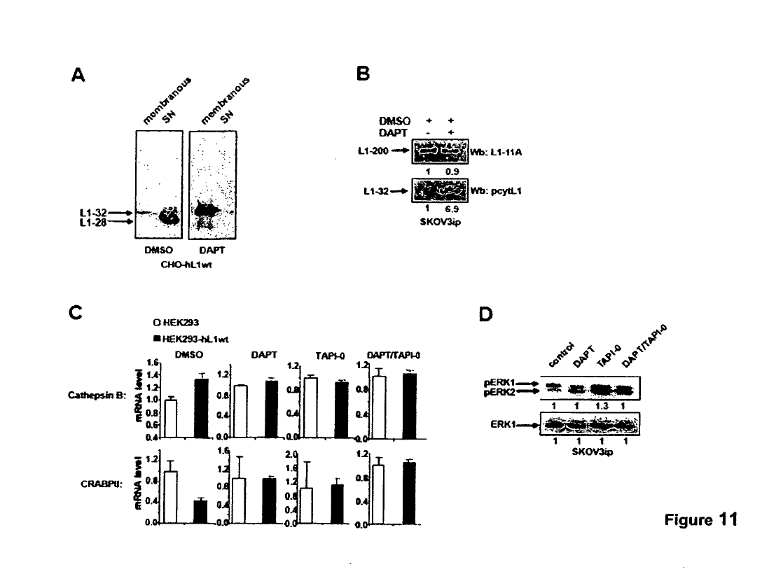

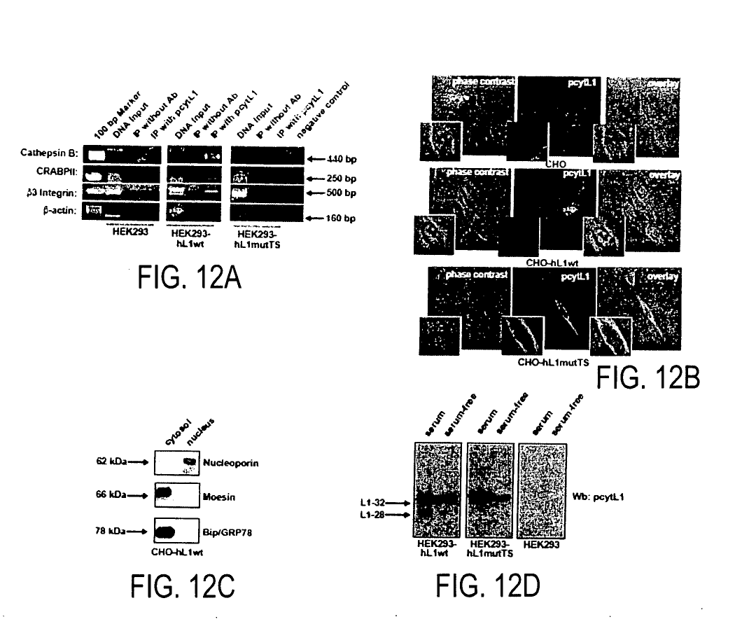

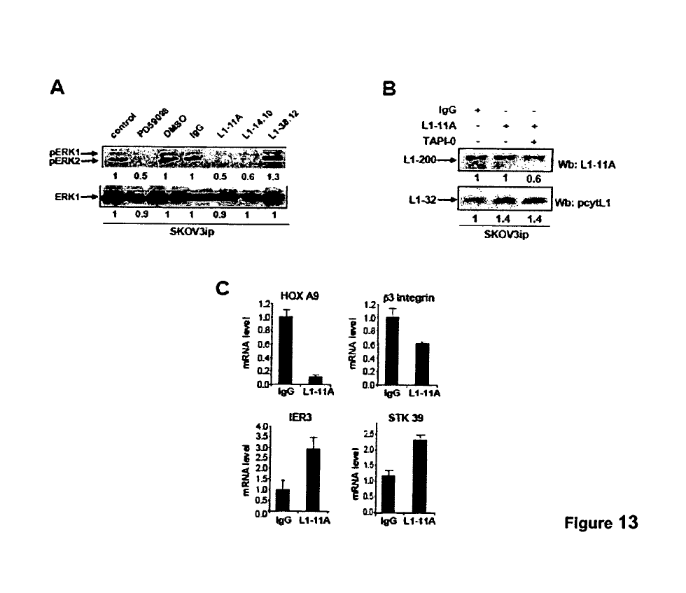

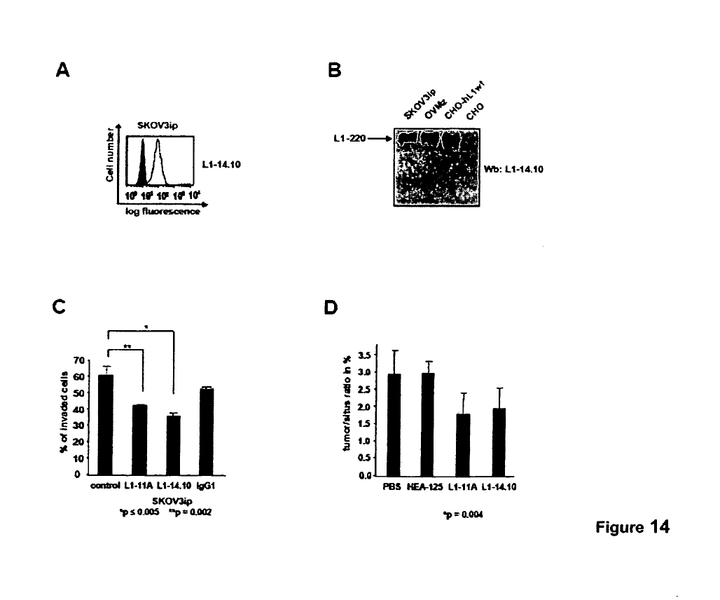

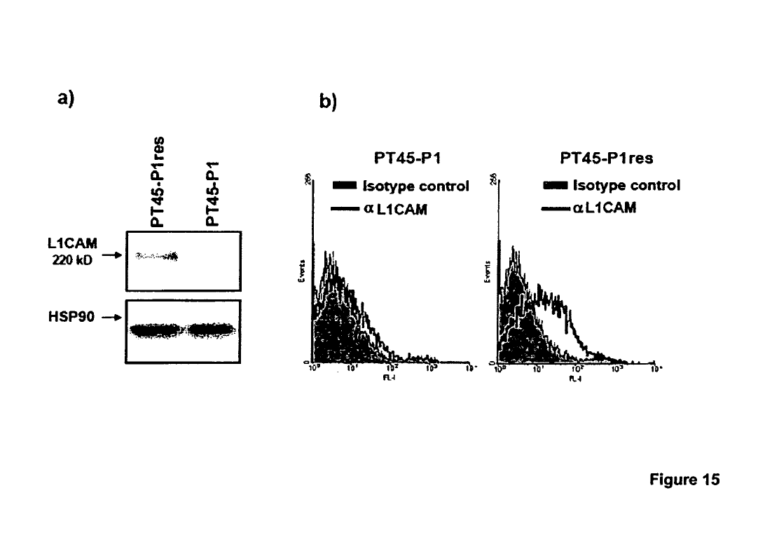

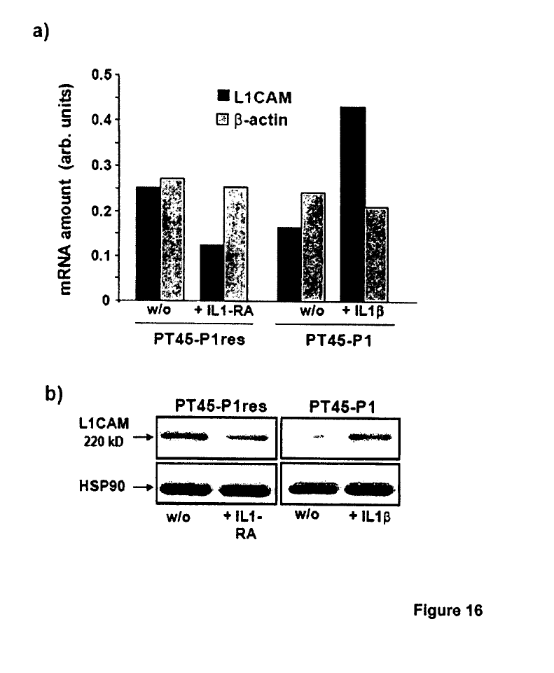

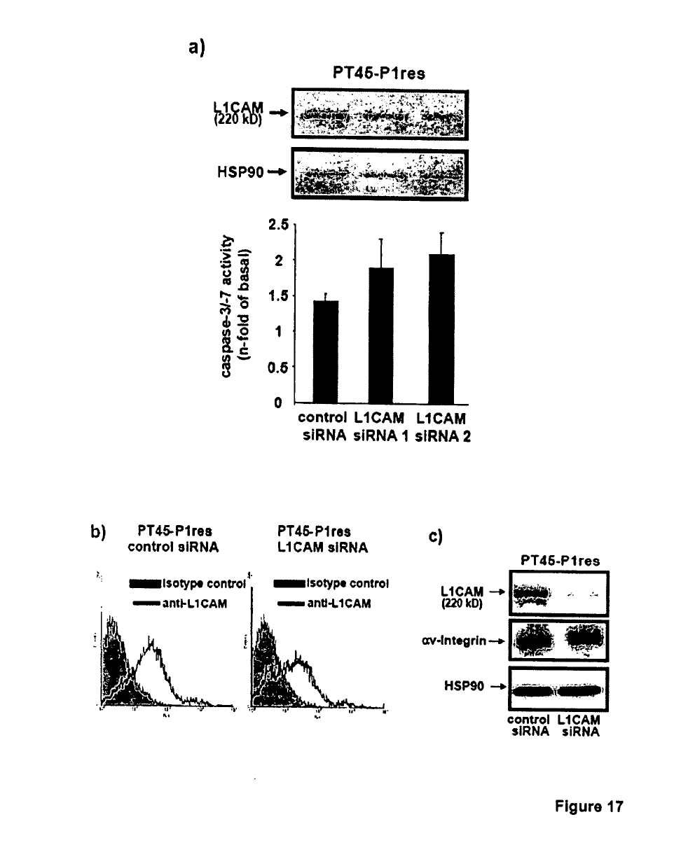

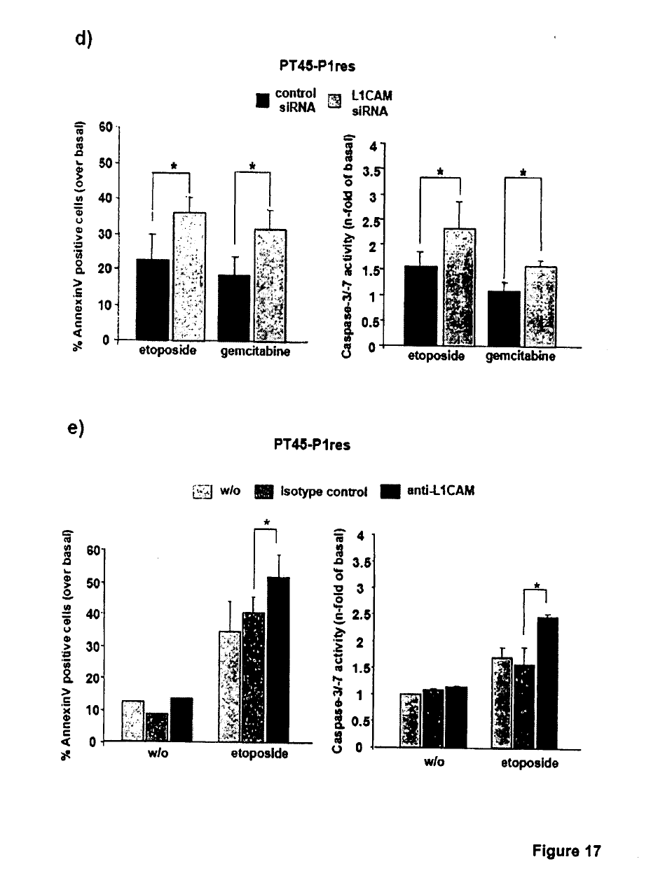

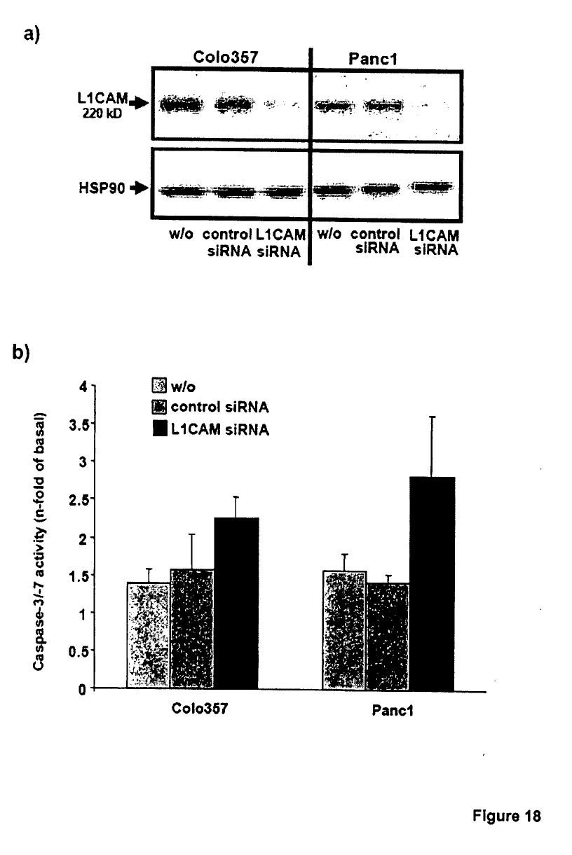

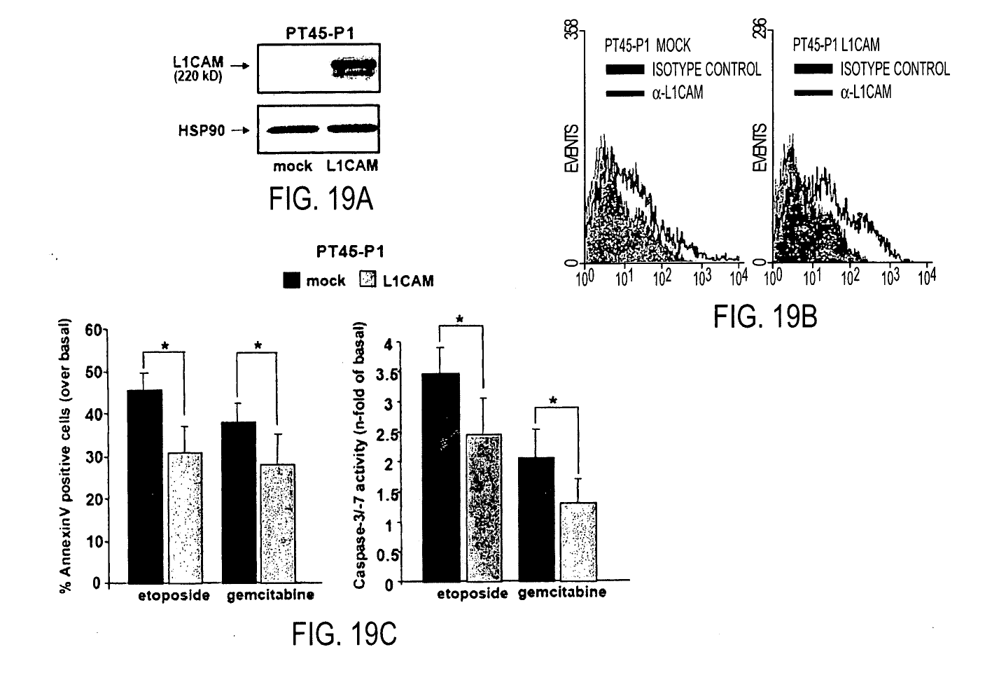

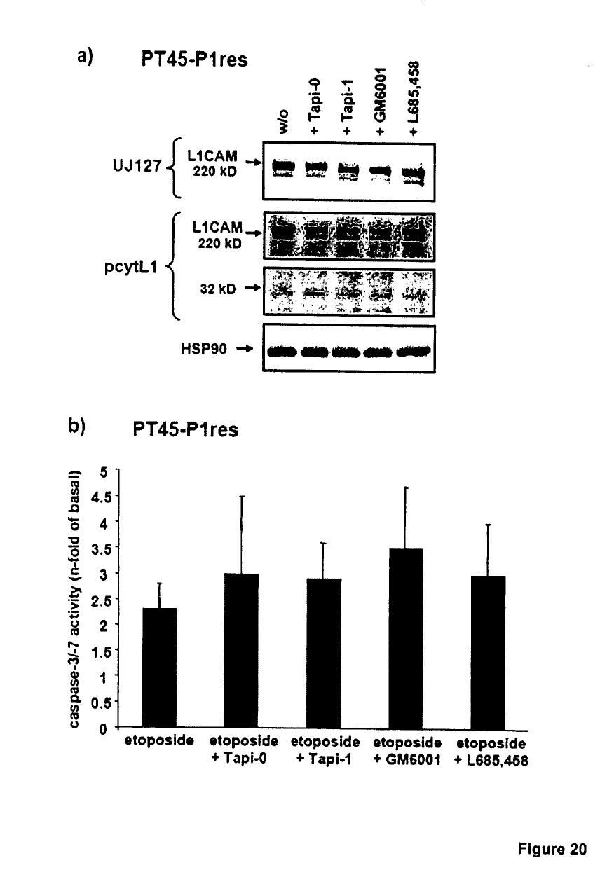

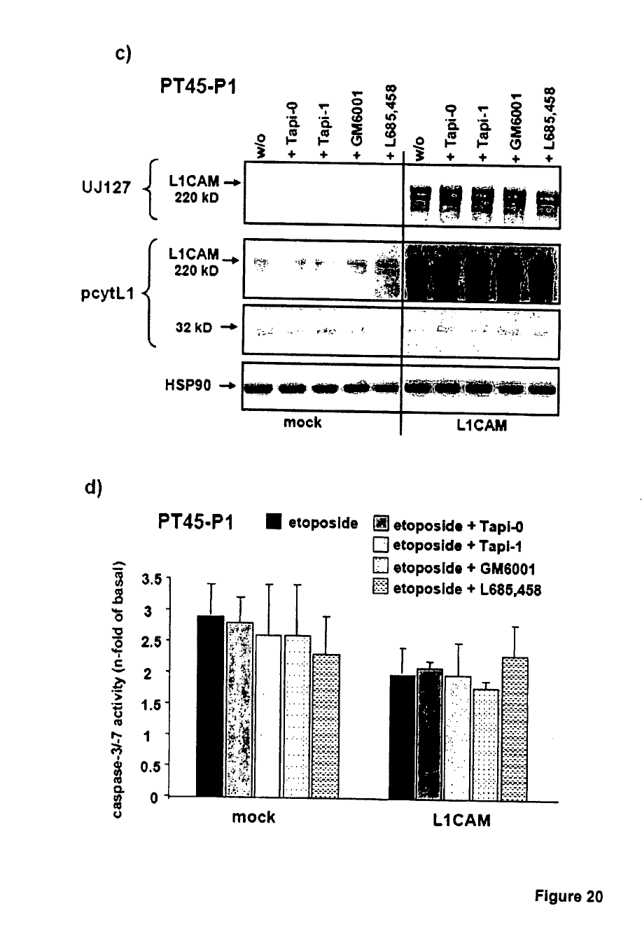

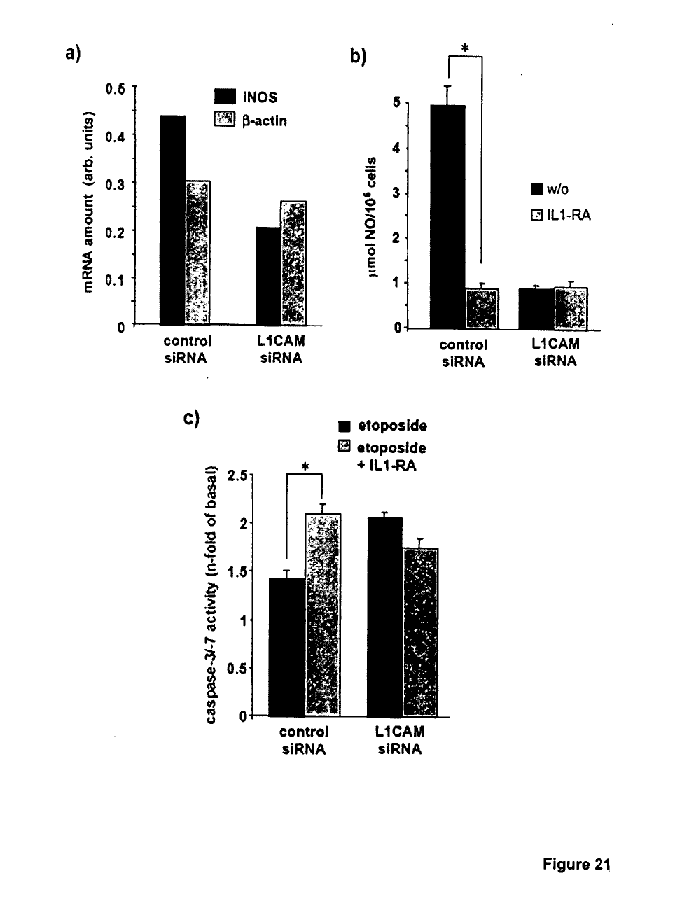

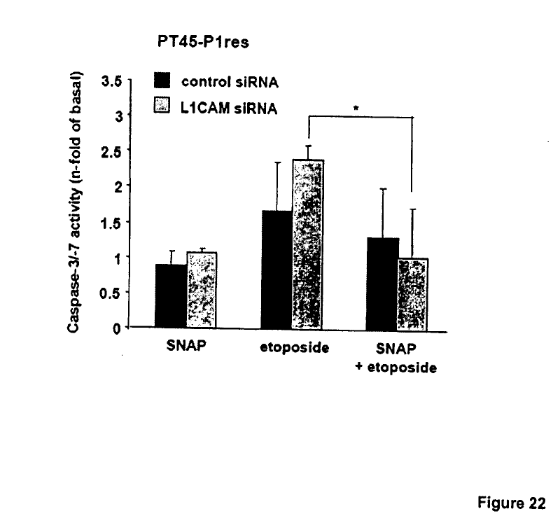

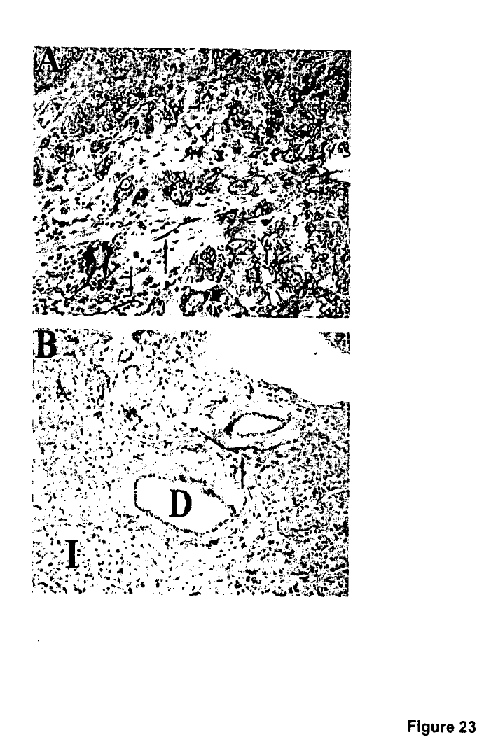

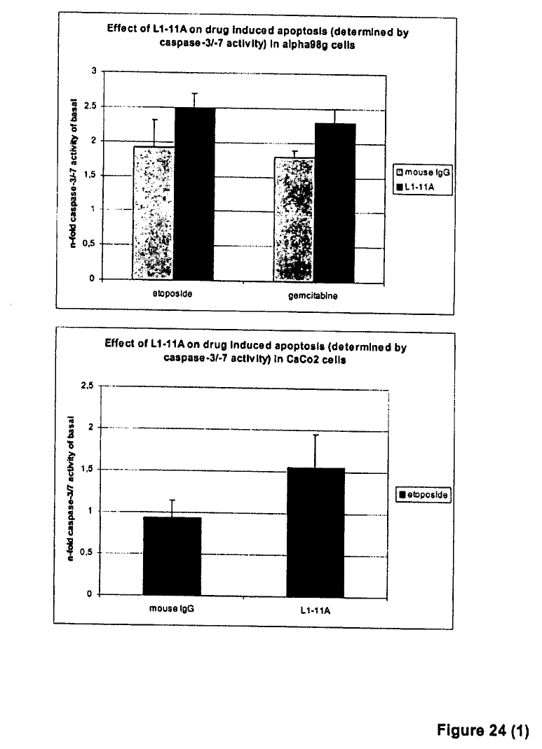

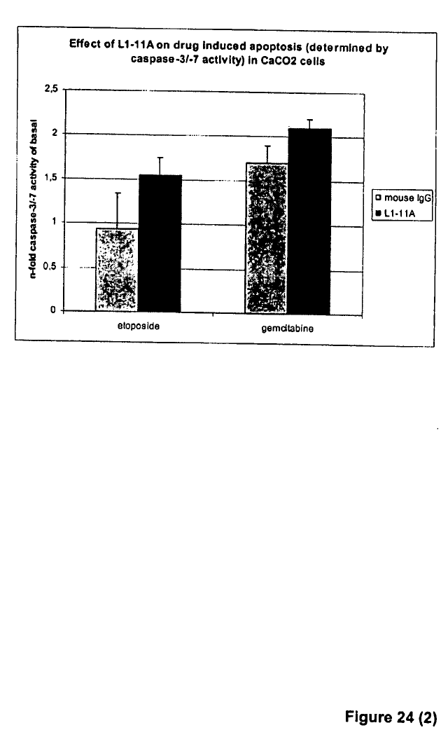

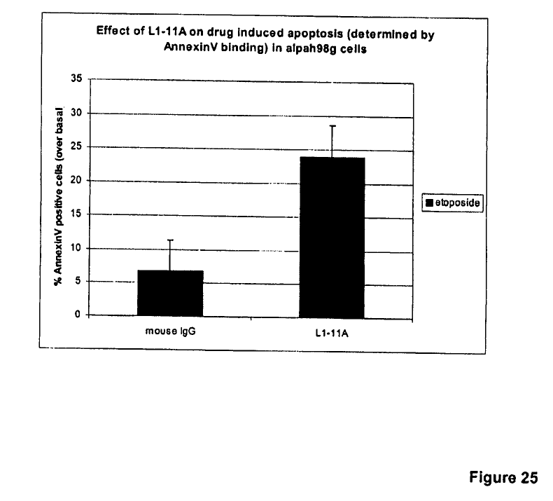

(A) Light microscopy of cisplatin treated and no-treated m130 cells. Note the more elongated morphology after treatment for 3 weeks. The bar represents 10 μm. (B) FACS analysis of cisplatin treated m130 cells with mAb L1-11A against human L1-CAM. The analysis was carried out as described in FIG. 1. (C) Western blot analysis of cell lysate from cisplatin treated on non-treated m130 cells. (D) FACS analysis of cisplatin treated SW707 colon carcinoma cells with mAb L1-11A against human L1-CAM.

FIG. 6. A role for L1 in gene regulation and apoptosis resistance

L1 expression in carcinomas leads to the production of soluble L1 due to metalloprotease-mediated cleavage by ADAM10 and ADAM17 [14,20,21]. Soluble L1 can bind to integrins such as α5β1 and αvβ5 and trigger ERK activation [23] leading to upregulation of Bcl-2. L1 expression itself can activate ERK via Src and is involved in transcriptional regulation including apoptosis-related genes [16,18]. L1-mediated gene regulation is dependent on ERK-activation [16,18] and L1 proteolytic processing by ADAMs and γ-secretase with subsequent nuclear translocation of the C-terminal fragment [18].

FIG. 7. Functional characterization of HEK293 cells expressing hL1wt and mutant L1

(A) A schematic view of the structure of L1. Mutant L1 forms containing changes in T1247A and S1248A site in the cytoplasmic portion is shown. (B) FACS analysis of stably transfected HEK293 cells. (C) Analysis of haptotactic cell migration. Fibronectin or BSA for control were coated onto the backside of Transwell chambers. The indicated stably transfected HEK293 cells were seeded into the top chamber and allowed to transmigrate. The migration of empty vector transfected cells (HEK293-mock) was set to 100%. (D) Analysis of matrigel cell invasion. Stably transfected HEK293 cells were seeded into a 6-well plate and allowed to invade into matrigel. (E) Tumor growth in mice. 10⁷ transfected HEK293 cells were injected into the left or right flanks of 6-week-old NOD/SCID mice, respectively. Tumor growth was monitored for 22 days, at which point the experiment was terminated. Sizes of tumors were measured with callipers and tumor volume was calculated. Results shown represent mean tumor volume for n=8 animals.

FIG. 8. Biochemical analysis of hL1wt or hL1mutTS expressing cells

(A) L1 processing and cleavage in transfected HEK293 cells. The cell lysates were analyzed by Western blot with pcyt-L1 recognizing the cytoplasmic portion of L1. The nomenclature of L1-cleavage fragments is according to a previous publication (Mechtersheimer et al, 2001). (B) ELISA analysis. Soluble L1 levels in the medium of HEK293-hL1wt or HEK293-hL1mutTS cells treated with or without PMA stimulation for 1 h at 37° C. was analyzed. Lysates from both cell lines were used as positive controls. (C) Stimulation of cell migration on fibronectin by recombinant L1-Fc. The fusion protein was added at the final concentration of 0.6 μg/ml. (D) Stimulation of cell migration by HEK293-hL1mutTS or HEK293-hL1wt supernatant containing soluble L1. Conditioned medium was concentrated ten-fold and used to stimulate the haptotactic cell migration of HEK293. (E) Phosphorylation of ERK1/2, FAK, PAK 1 and Src was analyzed in HEK293, HEK293-hL1wt and HEK293-hL1mutTS cells grown in serum. Relative band intensities as revealed by densitometric scanning are shown. (F) In vitro phosphorylation of GST-fusion proteins. The indicated fusion proteins were incubated with recombinant kinases and ³²P labeled γ-ATP. Labelled proteins were detected by autoradiography.

FIG. 9. hL1mutTS-mediated suppression of cell migration and invasion

(A) HEK293 cells or HEK293-hL1wt cells were transfected transiently with plasmids (10 μg DNA) encoding hL1mut, dominant-negative ADAM10 (ADAM10-DN) or empty pcDNA3 vector. Control transfection with EGFP-plasmid showed >50% transfection efficiency. 48 h after transfection, cells were analyzed for haptotactic cell migration on fibronectin. Each determination was done in quadruplicates. The MEK specific inhibitor PD59098 was used at a final concentration of 20 μM. (B) Adenoviral transduction of KS carcinoma cells with hL1wt and hL1mutTS. Cells were infected with a predetermined amounts of adenovirus (opu/cell). 48 h after transduction with hL1wt or hL1mutTS adenovirus or YFP-TM adenovirus for control, KS cells or the L1 positive ovarian carcinoma cells OVMz, SKOV3ip and MO68 were analyzed for haptotactic cell migration on fibronectin as described in the legend to FIG. 1. (C) The ovarian carcinoma cell lines OVMz and SKOV3ip were transduced with adenovirus as described above and analyzed for matrigel invasion. (D) ERK1/2 phosphorylation in OVMz cells was analyzed 48 h after transduction with the indicated adenoviral vectors. Relative band intensities as revealed by densitometric scanning are shown.

FIG. 10. L1-dependent gene regulation analysed by quantitative PCR

(A) Differential gene expression in HEK293, HEK293-hL1wt or HEK293-hL1mutTS cells. mRNAs from cells grown in serum were isolated, transcribed to cDNA and used as template for qPCR (SYBRgreen analysis). The indicated target genes were selected after initial gene chip analysis. Identification of differentially expressed proteins (B) by Western blot analysis using antibodies to cathepsin B and CRABPII with actin as loading control and (C) by FACS analysis with antibodies to the β3 integrin subunit, the αvβ3 integrin and cathepsin B. Note that αvβ5 expression is unaltered and that only small amounts of cathepsin B are detectable at the cell surface. (D) RA inhibits in vitro growth of HEK293 and HEK293-hL1mutTS but not HEK293-hL1wt cells.

FIG. 11. Requirement of metalloprotease and presenilin cleavage in L1-mediated gene regulation

(A) Analysis of L1-32 cleavage by γ-secretase. Cells were treated for 48 h at 37° C. with presenilin inhibitor IX (DAPT) or for control with DMSO. Isolated membranes were incubated for 2 h at 37° C. and then separated into pellet or supernatant (SN) fractions by ultracentrifugation. Lanes 1 to 2 show cells treated with DMSO (vehicle). Lanes 3 and 4 show cells preincubated with DAPT. (B) SKOV3ip cells were treated with DAPT either in the presence or absence of the metalloprotease inhibitor TAPI-0 for 24 hr. Cells were lysed in BOG lysis buffer and analyzed by Western blot analysis. The cell supernatant was analyzed for soluble L1 using mAb L1-11A and the cell lysate was examined for L1-32 using peytL1. (C) HEK293 or HEK293-hL1wt cells were treated with DMSO, DAPT, TAPI-0 or both inhibitors for 96 h. mRNA was transcribed to cDNA and analyzed by qPCR for the genes CRABPII and cathepsin B. (D) Analysis of ERK phosphorylation in SKOV3ip cells after treatment with the indicated compounds.

FIG. 12. Nuclear translocation of L1-CTF

(A) Analysis of L1-nuclear translocation by ChIP assay. Soluble chromatin was prepared from the indicated cell lines and immunoprecipitated with pcytL1. The final DNA extractions were amplified by PCR using pairs of primers that cover the promoter region of the indicated genes. An aliquot of extracted DNA was used as input control. (B) Nuclear localization of L1-CTF in CHO-hL1wt cells (middle row) and CHO-hL1mutTS cells (bottom row) L1 negative CHO cells were used as control (top row). Cells were fixed with 3% paraformaldehyde, permeabilised with methanol (−20° C.) and stained with pcytL1 and Alexa488-conjugated anti rabbit IgG. (C) Purity of isolated nuclei as revealed by marker protein analysis. (D) Presence of L1-CTF in the nucleus. HEK293, HEK-hL1wt or HEK-hL1mutTS cells were cultivated in the presence of 10% FCS or in serum free medium for 24 hr and nuclei were prepared and nuclear fragments were analyzed with pcyt-L1 and Western blot.

FIG. 13. Analysis of L1 antibody effects in vitro

(A) Effect of L1-antibodies on ERK phosphorylation in SKOV3ip cells. The cells were incubated for 24 hr at 37° C. with the indicated purified antibodies to L1 (10 μg/ml) or isotype control IgG. The mAb L1-38.12 recognizes only the neural form of human L1 but not the tumor form. Cells were also treated with DMSO (vehicle), or the ERK-specific inhibitor PD59098. Cell lysates were examined for phosphorylation of ERK. (B) Cells were treated with mAb L1-11A or isotype control IgG in the absence or presence of TAPI-0 as described above. Soluble L1 in the supernant and L1-32 in the cell lysate were analyzed by Western blot. (C) mRNA was isolated from antibody treated SKOV3ip cells, transcribed to cDNA and analyzed by qPCR for the indicated genes.

FIG. 14. Analysis of L1 antibody effects on invasion and tumor growth in mice

Characterization of the novel L1 mAb L1-14.10. (A) Fluorescence staining of SKOV3ip cells and FACS analysis. (B) Western blot analysis of cellular lysates from CHO, CHO-hL1wt, SKOV3ip and OVMz cells under reducing conditions. Full-length L1-220 is indicated. (C) SKOV3ip cells in the presence of the indicated L1 mAb (10 μg/ml) were examined in matrigel invasion assay. (D) Tumor growth in nude mice. LacZ-tagged SKOV3ip cells were injected i.p. into nude mice and after tumor implantation animals were treated with the indicated L1 mAbs or control mAb to EpCAM (HEA-125). After 30 days the tumor volume was determined and is given as the ratio between X-Gal stained tumor mass and the total sinus. 6 animals were analyzed per group.

FIG. 15. L1CAM expression in PT45-P1res and PT45-P1 cells

(a) Cellular lysates from PT45-P1res and PT45-P1 cells were subjected to western blotting using an antibody for the detection of full-length L1CAM (clone UJ127 from Acris) or of HSP90 as control for equal protein load. (b) Representative histograms from L1CAM surface staining (using the L1-11A antibody) or from isotype control staining of PT45-P1res and PT45-P1 cells determined by fluorescence flow cytometry.

FIG. 16. L1 CAM expression in chemoresistant PT45-P1res cells is IL1β dependent

(a) PT45-P1res cells were either left untreated (w/o) or treated with 250 ng/mL IL1-RA for 6 hours. In parallel, PT45-P1 cells were either left untreated (w/o) or treated with 20 ng/mL IL1β for 6 hours. L1CAM mRNA levels were analysed by real-time PCR and compared with β-actin used as control. Data from duplicate measurements are expressed as amount of mRNA in arbitrary units. Results from one representative out of three experiments are shown. (b) Cellular lysates from PT45-P1res and PT45-P1 cells were subjected to western blotting using an antibody for the detection of full-length L1CAM. PT45-P1res cells were either left untreated (w/o) or treated with 250 ng/mL IL1-RA for 24 hours. In parallel, PT45-P1 cells were either left untreated (w/o) or treated with 20 ng/mL IL1β for 24 hours. In all experiments, a HSP90 antibody was used as a control for equal protein load.

FIG. 17. L1 CAM is involved in the mediation of chemoresistance in PT45-P1res cells

(a) PT45-P1 res cells were transfected with control siRNA or with two L1CAM specific siRNAs. Western blotting for the detection of full-length L1CAM or of HSP90 as a control for equal protein load was performed (upper panel). In parallel, siRNA transfected PT45-P1res cells were treated with 20 μg/mL etoposide or not for 24 hours and caspase-3/-7 activity was determined. (b) siRNA transfected PT45-P1res cells were subjected to L1CAM immunostaining (L1-11A antibody) or staining with an isotype matched control antibody followed by flow cytometry. One representative histogram is shown. (c) siRNA transfected PT45-P1res cells were analysed by western blotting for the detection of full-length L1 CAM, αv-integrin or HSP90 (d) After overnight siRNA transfection, cells were either left untreated or were either treated with 20 μg/mL etoposide or with 5 μg/mL gemcitabine for 24 hours, followed by either AnnexinV/PI staining and flow cytometry (AnnexinV positive cells over basal) or by caspase-3/-7 assay (n-fold induced caspase-3/-7 activity of basal). (e) PT45-P1res cells were either left untreated (w/o) or were treated with 20 μg/mL etoposide in the absence (w/o) or presence of either 5 μg/mL anti L1CAM antibody (Clone L1-11A) or 5 μg/mL isotype matched control antibody. After 24 hours, cells were analysed by AnnexinV/PI staining or by caspase-3/-7 assay. Means±SD from three independent experiments are shown. * indicates p<0.05.

FIG. 18. Knock down of L1CAM abolished chemoresistance in Colo357 and Panc1 cells

Colo357 and Panel cells were either left untransfected (w/o) or were transfected with control siRNA or with L1CAM specific siRNA. a) Western blotting for the detection of full-length L1CAM or of HSP90 as control was performed. b) Untransfected (w/o) or siRNA-transfected Colo357 and Panel cells were either left untreated or treated with 20 μg/mL etoposide for 24 hours followed by the analysis of caspase-3/-7 activity (expressed as n-fold induced caspase-3/-7 activity of basal). Means±SD from three independent experiments are shown.

FIG. 19. L1 CAM expression induced a chemoresistant phenotype in PT45-P1 cells

PT45-P1 cells were either transfected with an empty vector (mock) or with L1CAM. (a) Western blotting for the detection of full-length L1CAM or of HSP90 as control was performed. b) Transfected PT45-P1 cells were subjected to L1CAM immunostaining (L1-11A antibody) or staining with an isotype matched control antibody followed by flow cytometry. One representative histogram is shown (c) After overnight transfection, cells were either left untreated or were either treated with 20 μg/mL etoposide or with 5 μg/mL gemcitabine for 24 hours followed by either AnnexinV/PI staining and flow cytometry (AnnexinV positive cells over basal) or by caspase-3/-7 assay (n-fold induced caspase-3/-7 activity of basal). Means±SD from three independent experiments are shown. * indicates p<0.05.

FIG. 20. L1 CAM cleavage is dispensable for induction of chemoresistance in PT45-P1 cells

PT45-P1res cells (a,b) or PT45-P1 cells transfected with L1CAM or an empty control vector (mock) (c,d) were left untreated (w/o) or were either treated with Tapi-0, Tapi-1, GM6001 or L685,458 (each 10 μmol/L) for 24 hours. (a,c) Cellular lysates were subjected to western blotting using either the antibody clone UJ127 from Acris detecting only full-length L1CAM or the pcytL1 antibody detecting also the cytoplasmic part of L1CAM. HSP90 was detected as a control for equal protein load. (b,d) Thirty minutes after treatment with the respective inhibitor, cells were either left untreated or treated with 20 μg/mL etoposide for 24 hours. Then, cells were analysed for caspase-3/-7 activity expressed as n-fold induction of caspase-3/-7 activity of basal. Means±SD from three independent experiments are shown. * indicates p<0.05.

FIG. 21. L1 CAM mediates iNOS induction and NO release in PT45-P1res cells

PT45-P1res cells were transfected with a control siRNA or with a L1CAM specific siRNA. (a) RNA from transfected PT45-P1res cells was subjected to RT and subsequent Real-time PCR using primers specific for iNOS. In parallel, a Real-time PCR was conducted for β-actin, which was used as a control. Results from one representative out of three experiments are shown. Data are expressed as amount of mRNA in arbitrary units. Each sample was measured in duplicates. (b) siRNA transfected cells (16 h) were either left untreated or were treated with 250 ng/mL IL1RA for 24 hours. Then, supernatants were cleared and subjected to a commercial NO assay. The amount of NO was normalized to equal cell number which was determined in parallel (expressed as μmol NO/10⁵ cells). (c) siRNA transfected cells (16 h) were either left untreated or were treated with 250 ng/mL IL1-RA and 20 μg/mL etoposide, either alone or in combination for 24 hours. Then, cells were analysed for caspase-3/-7 activity expressed as n-fold induced caspase-3/-7 activity of basal. Means±SD from three independent experiments are shown. * indicates p<0.05.

FIG. 22. Chemoresistance of PT45-P1res cells depends on L1CAM mediated NO secretion

PT45-P1res cells were transfected with a control siRNA or with a L1CAM specific siRNA. After overnight transfection, cells were either left untreated or were treated with 200 μmol/L SNAP, 20 μg/mL etoposide or with a combination of both. After 24 hours, cells were analysed for caspase-3/-7 activity expressed as n-fold induced caspase-3/-7 activity of basal. Means±SD from three independent experiments are shown. * indicates p<0.05.

FIG. 23. L1 CAM expression in pancreatic ductal adenocarcinoma

(a) Representative picture of moderate cytoplasmic and membrane-bound staining of L1 CAM in a poorly differentiated ductal adenocarcinoma. Small nerves served as internal positive control (arrows). (b) Representative picture of normal peritumoral pancreas tissue to in which only small nerves (arrows) express L1CAM (1-islet, D-duct).

FIG. 24. Effect of L1-11A on drug induced apoptosis (determined by caspase-3/-7 activity) in alpha98g cells and in CaCo2 cells

a98g and CaCO2 cells, respectively, were either left untreated or were treated with 20 μg/mL etoposide or with 5 μg/ml gemcitabine in the presence of either 5 μg/mL isotype matched control antibody (mouse IgG) or 5 μg/mL anti L1CAM antibody (Clone L1-11A). After 24 hours, cells were analysed by caspase-3/-7 assay. Data are expressed as n-fold caspase-3/-7 activity of basal. Means±SD from three independent experiments are shown. * indicates p<0.05 when comparing mouse IgG treated versus L1-11A treated cells.

FIG. 25. Effect of L1-11A on drug induced apoptosis (determined by AnnexinV binding) in alpha98g cells

a98g cells, respectively, were either left untreated or were treated with 20 μg/mL etoposide in the presence of either 5 μg/mL isotype matched control antibody (mouse IgG) or 5 μg/mL anti L1CAM antibody (Clone L1-11A). After 24 hours, cells were analysed by AnnexinV/PI staining and flow cytometry (expressed as % AnnexinV positive cells over basal).

### EXAMPLE 1

### Abstract

Objective. Apoptosis resistance is a hallmark of cancer progression, a phenomenon frequently observed in ovarian carcinoma. We reported previously, that L1 adhesion molecule (CD171) is overexpressed in ovarian and endometrial carcinomas and that L1 expression is a predictor of poor outcome. We investigated a possible role of L1 in apoptosis resistance.

Methods. We used L1 transfectants and ovarian carcinoma cell lines and induced apoptosis by different stimuli such as C2-Ceramide, staurosporine, cisplatin or hypoxia.

Results. We found that cells expressing L1 are more resistant against apoptosis. In HEK293 cells, L1-expression lead to a sustained ERK, FAK and PAK phosphorylation. Soluble L1 only partially rescued HEK293 cells from apoptosis. Treatment with apoptotic stimuli upregulated the anti-apoptotic molecule Bcl-2 to a greater extend in HEK293 cells expressing L1. In the ovarian carcinoma cell line OVMz, the depletion of L1 by RNA interference sensitized cells for apoptosis induction. No changes in activation of ERK or FAK were observed after L1 knockdown. The selection of m130 ovarian carcinoma or SW707 colon carcinoma cells with cisplatin lead to upregulated expression of L1.

Conclusions. Our results suggest a link between L1 expression and chemoresistance of ovarian carcinomas. Upregulation of L1 after cisplatin treatment might indicate a more malignant tumor phenotype given the established role of L1 in cell motility and invasion.

### Introduction

The acquisition of apoptosis resistance is a hallmark of cancer progression. In ovarian carcinoma, this is frequently observed. Chemotherapy is important in controlling residual disease following cyto-reductive surgery and as neo-adjuvant therapy in patients with advanced disease [1]. The standard chemotherapy for advanced ovarian cancer is currently paclitaxel-carboplatin or paclitaxel-cisplatin which is routinely given together with dexamethasone, a synthetic corticoid [2]. Despite initial response to therapy, ovarian carcinomas often aquire resistance to chemotherapeutic drugs leading to tumor recurrance and frequent death of the patients [1,2]. A better understanding of molecular mechanisms underlying chemoresistance is urgently needed. L1 is a type I membrane glycoprotein of 200-220 kDa structurally belonging to the Ig-superfamily [3]. L1 plays a crucial role in axon guidance and cell migration in the developing nervous system [4,5]. Recent studies have also implicated L1 expression in the progression of human carcinomas. L1 expression was found on different tumors including lung cancer [6], gliomas [7], melanomas [8,9], renal carcinoma [10,11], and colon carcinoma [12]. We reported before that L1 is overexpressed in ovarian and endometrial carcinomas in a stage-dependent manner and that L1 expression was a predictor of poor outcome [13]. A clear mechanism by which L1 expression could contribute to the progression of human tumors is still missing. However, several recent studied have shown that over-expression of L1 can augment cell motility of carcinoma cells on extracellular matrix proteins [14-16], and invasiveness in matrigel invasion assays [12,17,18]. L1 expression was also found to enhance tumor growth in NOD/SCID mice [12,19] and was found to induce L1-dependent gene expression [16,18]. We demonstrated before that L1 is released from the cell membrane by the metalloproteases ADAM10 [14,20] and ADAM17 [21,22]. The soluble L1 ectodomain, as a product of L1 cleavage, is detectable in serum and ascites from ovarian carcinoma patients [13]. Soluble L1 from ascites is a potent inducer of cell migration [23]. Other functions, for instance in apoptosis protection, have not been investigated. In the present communication, we have addressed the question if expression of L1 and/or soluble L1 has an influence on apoptosis of ovarian carcinoma cells. We used cell lines stably transfected with L1 and ovarian carcinoma cell lines to study the influence of L1 expression on apoptosis induced through different apoptotic stimuli including C2-Ceramide, staurosporine, cisplatin or hypoxic conditions. Our results show that expression of L1 affected apoptosis sensitivity and suggests a link between L1 expression and chemoresistance of ovarian carcinomas.

### Material and Methods

### Cells

The ovarian carcinoma cell lines OVMz and m130 have been described before [19,20]. The human epithelial kidney cell line HEK293 and the chinese hamster ovary (CHO) cell line stably expressing human L1 (hL1) were established by transfection with superfect (Stratagene, Heidelberg, Germany) and selection for L1 expression with mAb L1-11A and magnetic beads (Miltenyi Biotec, Bergisch Gladbach, Germany) or sorting by FACS as described before [19,20]. All cells were cultivated in DMEM supplemented with 10% FBS at 37° C., 5% CO2 and 100% humidity. Experiments with human material were approved by the Ethical committee of the University of Heidelberg.

### Chemicals and Antibodies

Antibodies to the ectodomain (mAb L1-11A, subclone of mAb UJ 127.11) or cytoplasmic domain (pcytL1) of human L1 have been described (10). Antibodies to ERK1, phospho-ERK1/2, FAK and phosphor-FAK (p125) were purchased from BD-Transduction (Heidelberg, Germany). The antibody to phospho-PAK1 was purchased from Cell Signaling (New England Biolabs, Frankfurt, Germany). The antibody against Bcl-2 was from Santa Cruz (Heidelberg, Germany). Secondary antibodies were obtained from Dianova (Hamburg, Germany). Cell permeable C2-ceramide, staurosporine and cis-Diammineplatinum(II) dichloride (cisplatin) were purchased from Sigma (Taufkirchen, Germany). Triton X-100 was from Gerbu (Gaiberg, Germany).

Induction of Apoptosis in Cell Cultures

Cells (1×10⁵ cells per well) were cultured in duplicate in 6 well culture plates and, after washing once with PBS, incubated for the indicated timepoints with or without apoptotic stimuli in serum-free medium. Staurosporine (2 μM), C2-ceramide (40 μM) or cisplatin (17 μM) were used as apoptotic reagents. For hypoxia studies, cells were incubated for different time points under hypoxic conditions (1% O2) using a Reming Bioinstruments chamber and oxygen regulator (Reming Bioinstruments, Redfield, N.Y.).

### Assessment of Apoptotic Cell Death and FA CS Analysis

For quantification of DNA fragmentation, supernatants were centrifuged at 200×g. The cell pellet was washed once in PBS (pH 7.4) and lysed in a hypotonic lysis buffer (0.1% sodium citrate, 0.1% Triton X-100, 50 μg/ml propidiumiodite) at 4° C. overnight. The nuclei were then analyzed for DNA content by flow cytometry [24]. The staining of cells for flow cytometry with mAbs and PE-conjugated secondary antibodies has been described [14]. Cells were analysed with a FACScan using Cellquest or FlowJo software from Becton Dickinson (Heidelberg, Germany).

### Biochemical Analysis and Isolation of Soluble L1

SDS-PAGE under reducing conditions and transfer of separated proteins to Immobilon membranes using semi-dry blotting was described before [25]. After blocking with 5% skim milk in TBS, the blots were developed with the respective primary antibody followed by peroxidase conjugated secondary antibody and ECL detection. The isolation and characterization of soluble L1 from ascites fluid was described before [23]. Briefly, the vesicle cleared ascites fluid from ovarian carcinoma patients was adsorbed to Sepharoselinked mAb L1-11A and bound L1 was eluted with 0.1 M Glycin/HCl buffer pH 2.8. Eluted fractions were neutralized and an aliquot of the samples was analyzed by SDS-PAGE. Cell pellets were lysed in lysis buffer (20 mM Tris/HCl pH 8.0 containing 1% Triton X-100, 150 mM NaCl, 1 mM PMSF), cleared by centrifugation and mixed with two fold-concentrated reducing SDS-sample buffer.

### SiRNA Transfection

Transfection of siRNAs was described before [22]. L1 (5′-AGGGAUGG-UGUCCACCUUCAAAUU-3′) siRNA (SEQ ID NO: 1) was synthesized by MWG-Biotech (Ebersberg, Germany). Cells were transfected with annealed siRNAs using Oligofectamine (Life Technologies) and analyzed after the indicated time points.

### Statistical Analysis

For the analysis of statistical significance the student's t-test was used.

### Results

### Expression of L1 Enhances Apoptosis Resistance in HEK293 Cells

We initially analyzed the role of L1 in apoptosis resistance using stably transfected cell lines. HEK293 and HEK293-hL1 cells were treated with C2-ceramide or staurosporine under serum-free conditions and apoptosis was analyzed by Nicoletti staining. L1-expressing cells were more resistant against apoptosis induced through both stimuli (FIGS. 1B and C). 93% of the cells were still viable after 24 h treatment with C2-ceramide, compared to only 63% of wild-type HEK293 cells. Similar differences were observed at later time points (FIG. 1B) or after treatment with staurosporine. Under these conditions, 77% of L1-expressing cells were viable in contrast to only 59% of the parental cells. No differences in the viability could be observed under serum free conditions after 24 hours (FIGS. 1B and C). Cancer cells are often resistant to chemotherapeutic agents due to protection from apoptotis. To study a possible influence of L1 expression on apoptosis induced through the chemotherapeutic drug cisplatin, we treated HEK293 and HEK293-hL1 cells for various length of time. Under these conditions, L1 expressing cells again showed a more resistant phenotype than L1 negative cells. Approximately 70% of L1 positive cells survived the treatment with cisplatin as opposed to only 40% of L1-negative cells (FIG. 1D). These experiment showed a clear correlation between L1 expression, and apoptosis resistance/chemoresistance of treated cells. In malignant tumors, the rate of apoptosis is high in under-vascularized areas. It is known that low oxygen pressure or hypoxia can directly induce apoptosis of tumor cells. We used hypoxic conditions as a physiological inducer of apoptosis in HEK293 and HEK293-hL1 cells and compared the rate of apoptosis. Approximately 30% more L1 positive than negative cells survived hypoxic conditions. Although the incubation of both cell lines under serum-free conditions for 48 hours induced cell death, no differences could be observed between both cell lines. To confirm these data for another cell line, we compared the rate of apoptosis of CHO and CHO-hL1 cells under hypoxic conditions (FIG. 1E, left). At all time points, there were significantly more L1 positive CHO cells alive than negative ones (FIG. 1E, right). These findings indicated that the effects of L1 expression on apoptosis resistance could also be observed in CHO cells.

### Expression of L1 Alters FAK, PAK and ERK Phosphorylation in HEK293 Cells

Some L1 functions are known to involve ERK1/2 activation [15,26]. Indeed, a recent study has demonstrated that L1 expression leads to sustained ERK activation, leading to enhanced motility of cells and augmented activity of ERK1/2-dependent genes [12,16,19]. Indeed, we observed that ERK and FAK were phosphorylated in L1 expressing HEK293 cells (FIG. 2A). L1-negative cells showed lower FAK activation. PAK 1, a downstream target of FAK, was also activated in L1 expressing cells (FIG. 2A). A recent study showed that L1-mediated neuroprotection involved enhanced Bcl-2 expression [27]. Indeed, it has also been established that triggering via integrins upregulates Bcl-2 expression [28]. We examined this question for HEK293 cells. Indeed, as shown in FIG. 2B, whereas the normalized levels of Bcl-2 protein were quite similar without apoptotic stimulus, these values became significantly higher in L1 expressing cells after induction of apoptosis.

### Soluble L1 has Little Protective Effect in HEK293 Cells

It is known that soluble L1 can stimulate cell migration and trigger ERK-phosphorylation by binding to integrins [23]. In addition, the release of soluble L1 is increased by apoptotic stimuli [23]. Therefore, we investigated the role of soluble L1 on apoptosis protection. We incubated HEK293 and HEK293-hL1 cells in the absence or presence of purified L1 for 12 hours with staurosporine to induce apoptosis. As shown in FIG. 3A, soluble L1 enhanced survival of both cell lines to a similar degree. Importantly, soluble L1 could only partially rescue HEK293 cells from apoptosis and the rate was not increased when higher amounts of soluble L1 were added (data not shown). Chen et al. also demonstrated, that soluble L1 triggers FAK phosphorylation [27]. We looked for the effect of soluble L1 on FAK phosphorylation in HEK293 cells and observed that the addition of soluble L1 indeed enhanced FAK phosphorylation (FIG. 3B). Collectively, the results suggested that soluble L1 only partially contributed to apoptosis protection and that neural and carcinoma cells showed similar responses to soluble L1.

Knockdown of L1 with siRNA Sensitizes OVMz Cells to Apoptosis

We extended our analysis to ovarian carcinoma cell lines. In previous work, we investigated a panel of L1 positive and L1 negative ovarian carcinoma cell lines for apoptosis resistance towards cisplatin [29]. For further analysis, we chose the L1 high expressing cell line OVMz (IC50 for cisplatin >14 μM) and the L1 negative cell line m130 (IC50 for cisplatin=7 μM) [29]. To further examine the role of L1 expression in apoptotic resistance, we used siRNA-mediated knockdown of L1 in OVMz cells. As detected by FACS analysis, the L1-specific siRNAs caused a downregulation of L1 expression at the cell surface after 48 hours (FIG. 4A). The loss of L1 expression could also be observed by Western blot in cell lysates. We noticed a depletion of full-length L-220 and the cleavage fragments of L1-32 (FIG. 4B). Transfection of a control siRNA (si scr) had no effect on L1 expression. Importantly, when the phosphorylation of ERK and FAK was examined in L1-depleted OVMz cells, no significant reduction was observed (FIG. 4C). However, the sensitivity to apoptosis induced by cisplatin or staurosporine was significantly enhanced compared to control siRNA treated cells (FIG. 4D, E). In contrast, there was no difference in the rate of apoptosis under normal culture conditions or under serum-free conditions. These data suggested, that in OVMz cells, ERK activation is constitutive and not dependent on L1 expression but L1 seems to be essential for apoptosis protection.

Long-Term Cisplatin Treatment Augments L1 Expression in m130 Cells

We studied whether long-term cisplatin treatment of L1-negative m130 cells would augment L1 expression. M130 cells were treated cells with increasing amounts of cisplatin over a time period of 3 weeks (10 μM first week, 15 μM second week, 20 μM third week). The long-term treatment altered the morphological phenotype of the cells (FIG. 5A) and clearly decreased cell proliferation (data not shown). After three weeks of treatment, the expression of L1 was strongly enhanced as revealed by FACS (FIG. 5B, lower panel) and Western Blot analysis (FIG. 5C). Similar results were obtained in the L1 negative colon carcinoma cells SW707 (FIG. 5D). These results suggest that enhanced apoptosis resistance by continuous exposure to cisplatin augments the expression levels of L1.

### Discussion

High-grade ovarian carcinoma is a life-threatening disease with a low five-year survival rate. Currently, the preferred treatment regimen after surgery is combined chemotherapy comprising usually a platinum based drug, such as cisplatin or carboplatin, coupled with paclitaxel. While this treatment course shows promising effects in a high percentage of cases, the development of chemoresistance is a hurdle that significantly reduces successful treatment outcomes. We previously reported that the expression of L1-CAM is associated with poor outcome in ovarian and endometrial carcinomas [13]. Here we examined the effects of L1 expression on apoptosis and chemoresistance using transfected cell lines and established ovarian carcinoma cell lines. We observed that (i) the expression of L1 augments apoptosis resistance and chemoresistance in carcinoma cells; (ii) in HEK293 cells, enhanced resistance was accompanied by activation of ERK, FAK and enhanced expression of Bcl-2; (iii) soluble L1 was only partially able to rescue cells from apoptosis; (iv) in ovarian carcinoma cells the depletion of L1 sensitized cells for apoptosis and (v) selection for chemoresistance to cisplatin upregulated L1 expression. A role for L1 in protection from apoptosis was previously studied in neural cells. Cerebellar granule neurons of mouse and hippocampal neurons of the rat embryo undergo apoptosis when cultured in serum-free medium. Both the addition of soluble L1 in the form of an L1-FC fusion protein or the cultivation on L1-substrates could enhance the survival of neurons [27,30]. A similar effect of soluble L1 and the related molecule CHL1 was also shown for cultured purified motoneurons from E14 rat embryos [31]. A recent study by Loers at al. showed that L1-substrate triggered neuritogenisis and neuroprotection depended on distinct but overlapping signal transduction pathways [30]. It was found that inhibitors for PI3 kinase, src family kinases and the MAP-kinase pathway could block neuroprotection [30]. L1 promoted neuroprotection was associated with increased phosphorylation of ERK, Akt and Bad as well as inhibition of caspases [30]. Previous work in ovarian carcinoma cells has also linked activation of ERK and FAK to apoptosis protection [32]. Our results show that expression of L1 and the addition of soluble L1 can activate these signalling pathways in HEK293 cells. However, in OVMz ovarian carcinoma cells, ERK activation was independent of L1 as its depletion sensitized the cells to apoptosis induction without changing the activation of ERK and FAK.

In many ovarian carcinoma patients, considerable amounts of soluble L1 are present in ascites fluid and serum [13,23]. In the light of previous publications in neural cells [27,30], it was therefore conceivable to assume a role for soluble L1 in apoptosis protection for carcinoma cells. Our results show, that the addition of soluble L1 to HEK293 cells and to m130 cells (A. Stoeck, unpublished results) cannot rescue cells from apoptosis. The finding that in the presence of soluble L1, HEK293-hL1 cells were more resistant to apoptosis than non-transfected HEK293 cells, argued against a prominent role of soluble L1. It appears, that the membrane bound form of L1 is more efficient than soluble L1. If soluble L1 does not have this importance, is L1 cleavage from the membrane then indispensable?

It was shown before that metalloproteinase inhibitors, that block the ectodomain cleavage of membrane-bound L1 or the EGF receptor ligand HB-EGF, render cells susceptible to apoptosis induction [23,33]. Similarily, for both L1-promoted neuritogensis and neuroprotection, the proteolytic cleavage of L1 (or its interaction partners) was necessary [30]. Interestingly, our recent data have shown that ADAM-mediated ectodomain cleavage is instrumental for L1-mediated gene regulation [18]. A model for the function of L1 in tumors is emerging from the previous study and the results presented in this report that is depicted in FIG. 6. We observed that the initial ADAM10 cleavage product L1-32 is further processed by γ-secretase [30,18], can translocates to the nucleus and was identified by chromatin-IP in transcriptional complexes [18]. By differential hybridisiation and q-RT-PCR, we identified genes that are upregulated in HEK293-hL1 expressing cells in comparison to HEK293 cells.

We observed upregulation of genes such as cathepsin B, β3integrin and the transcription factors HOX A9 and AP2α. Interestingly, the apoptosis related gene Mdm 2 was also upregulated whereas downregulation was noticed for the retinoic acid binding protein CRABPII, and the apoptosis-inducing genes STK 39 and IER 3 [18]. Earlier data by Siletti had already indicated that L1 expression can cause up-regulation of the anti-apoptotic gene XIAP [30]. Thus, it is possible that L1-mediated gene regulation controls enhanced resistance to apoptosis and sensitivity to chemotherapeutic drugs. The depletion of OVMz cells with L1-specific siRNAs reduced full-length L1 and the ADAM10 cleavage product L1-32 and may thereby block L1-signalling. It remains to be investigated whether L1-mediated gene regulation is also operative in human ovarian carcinomas in situ.

Finally, our observation that long-term treatment with cisplatin upregulated L1 expression might be of some clinical relevance. If such a selection would happen also in situ during chemotherapy, it would enrich for tumor cells with enhanced motility, invasiveness and better growth characteristics. This would be of great disadvantage for the patient. Recent work has shown that antibodies to L1 have therapeutical potential and can reduce cell proliferation in vitro [12,34], and in vivo growth in a xenograft mouse model for human ovarian carcinoma [35]. Thus, L1 might be a novel target for antibody-based therapy as second line therapy against aggressive human ovarian tumors. It is feasible that upregulation of L1 by chemotherapeutic drugs like cisplatin might improve the targeting and efficacy of L1-antibodies.

### REFERENCES

[1] Cannistra S A. Cancer of the ovary. N Engl J Med 2004; 351:2519-29.
[2] Vergote I, van Gorp T, Amant F, Neven P, Berteloot P. Neoadjuvant chemotherapy for ovarian cancer. Oncology (Williston Park) 2005; 19:1615-22
[3] Moos M, Tacke R, Scherer H, Teplow D, Fruh K, Schachner M. Neural adhesion molecule L1 as a member of the immunoglobulin superfamily with binding domains similar to fibronectin. Nature 1988; 334:701-3.
[4] Hortsch M. Structural and functional evolution of the L1 family: are four adhesion molecules better than one? Mol Cell Neurosci 2000; 15:1-10.
[5] Schachner M. Neural recognition molecules and synaptic plasticity. Curr Opin Cell Biol 1997; 9:627-34.
[6] Katayama M, Iwamatsu A, Masutani H, Furuke K, Takeda K, Wada H, et al. Expression of neural cell adhesion molecule L1 in human lung cancer cell lines. Cell Struct Funct 1997; 22:511-6.
[7] Senner V, Kismann E, Puttmann S, Hoess N, Baur I, Paulus W. L1 expressed by glioma cells promotes adhesion but not migration. Glia 2002; 38:146-54. [8] Thies A, Schachner M, Moll I, Berger J, Schulze H J, Brunner G, et al. Overexpression of the cell adhesion molecule L1 is associated with metastasis in cutaneous malignant melanoma. Eur J Cancer 2002; 38:1708-16.
[9] Fogel M, Mechtersheimer S, Huszar M, Smimov A, Abu D A, Tilgen W, et al. L1 adhesion molecule (CD 171) in development and progression of human malignant melanoma. Cancer Lett 2003; 189:237-47.
[10] Meli M L, Carrel F, Waibel R, Amstutz H, Crompton N, Jaussi R, Moch H, Schubiger P A, Novak-Hofer I. Anti-neuroblastoma antibody chCE7 binds to an isoform of L1-CAM present in renal carcinoma cells. Int J Cancer, 1999; 83: 401-408.
[11] Allory Y, Matsuoka Y, Bazille C, Christensen E I, Ronco P, Debiec H. The L1 cell adhesion molecule is induced in renal cancer cells and correlates with metastasis in clear cell carcinomas. Clin Cancer Res 2005; 11:1190-7.
[12] Gavert N, Conacci-Sorrell M, Gast D, Schneider A, Altevogt P, Brabletz T, et al. L1, a novel target of beta-catenin signaling, transforms cells and is expressed at the invasive front of colon cancers. J Cell Biol 2005; 168:633-42.
[13] Fogel M, Gutwein P, Mechtersheimer S, Riedle S, Stoeck A, Smimov A, et al. L1 expression as a predictor of progression and survival in patients with uterine and ovarian carcinomas. Lancet 2003; 362:869-75.
[14] Mechtersheimer S, Gutwein P, Agmon L N, Stoeck A, Oleszewski M, Riedle S, et al. Ectodomain shedding of L1 adhesion molecule promotes cell migration by autocrine binding to integrins. J Cell Biol 2001; 155:661-74.
[15] Thelen K, Kedar V, Panicker A K, Sclmiid R S, Midkiff B R, Maness P F. The neural cell adhesion molecule L1 potentiates integrin-dependent cell migration to extracellular matrix proteins. J Neurosci 2002; 22:4918-31.
[16] Silletti S, Yebra M, Perez B, Cirulli V, McMahon M, Montgomery A M. Extracellular signal-regulated kinase (ERK)-dependent gene expression contributes to L1 cell adhesion molecule-dependent motility and invasion. J Biol Chem 2004; 279:28880-8.
[17] Meier F, Busch S, Gast D, Goppert A, Altevogt P, Maczey E, et al. The adhesion molecule L1 (CD171) promotes melanoma progression. Int J Cancer 2006; 119:549-55.
[18] Gast D, Riedle S, Issa Y, Beckhove P, Sanderson M P, Lemmon V, et al. The cytoplasmic portion of L1-CAM controls gene expression in tumor cells that is reversed by therapeutic L1 antibodies. 2006; submitted.
[19] Gast D, Riedle S, Schabath H, Schlich S, Schneider A, Issa Y, et al. L1 augments cell migration and tumor growth but not beta3 integrin expression in ovarian carcinomas. Int J Cancer 2005; 115:658-65.
[20] Gutwein P, Mechtersheimer S, Riedle S, Stoeck A, Gast D, Joumaa S, et al. ADAM10 mediated cleavage of L1 adhesion molecule at the cell surface and in released membrane vesicles. Faseb J 2003; 17:292-4.
[21] Maretzky T, Schulte M, Ludwig A, Rose-John S, Blobel C, Hartmann D, et al. L1 is sequentially processed by two differently activated metalloproteases and presenilin/gamma-secretase and regulates neural cell adhesion, cell migration, and neurite outgrowth. Mol Cell Biol 2005; 25:9040-53.
[22] Stoeck A, Keller S, Riedle S, Sanderson M P, Runz S, Le Naour F, et al. A role for exosomes in the constitutive and stimulus-induced ectodomain cleavage of L1 and CD44. Biochem J 2006; 393:609-18.
[23] Gutwein P, Stoeck A, Riedle S, Gast D, Runz S, Condon T P, et al. Cleavage of L1 in exosomes and apoptotic membrane vesicles released from ovarian carcinoma cells. Clin Cancer Res 2005; 11:2492-501.
[24] Nicoletti I, Migliorati G, Pagliacci M C, Grignani F, Riccardi C. A rapid and simple method for measuring thymocyte apoptosis by propidium iodide staining and flow cytometry. J Immunol Methods 1991; 139:271-9.
[25] Gutwein P, Oleszewski M, Mechtersheimer S, Agmon-Levin N, Krauss K, Altevogt P. Role of Src kinases in the ADAM-mediated release of L1 adhesion molecule from human tumor cells. J Biol Chem 2000; 275:15490-7.
[26] Schaefer A W, Kamiguchi H, Wong E V, Beach C M, Landreth G, Lemmon V. Activation of the MAPK signal cascade by the neural cell adhesion molecule L1 requires L1 internalization. J Biol Chem 1999; 274:37965-73.
[27] Chen S, Mantei N, Dong L, Schachner M. Prevention of neuronal cell death by neural adhesion molecules L1 and CHL1. J Neurobiol 1999; 38:428-39.
[28] Matter M L, Ruoslahti E. A signaling pathway from the alpha5beta1 and alpha(v) beta3 integrins that elevates bcl-2 transcription. J Biol Chem 2001; 276:27757-63.
[29] Zhang C, Marme A, Wenger T, Gutwein P, Edler L, Rittgen W, et al. Glucocorticoid-mediated inhibition of chemotherapy in ovarian carcinomas. Int J Oncol 2006; 28:551-8.
[30] Loers G, Chen S, Grumet M, Schachner M. Signal transduction pathways implicated in neural recognition molecule L1 triggered neuroprotection and neuritogenesis. J Neurochem 2005; 92:1463-76.
[31] Nishimune H, Bernreuther C, Carroll P, Chen S, Schachner M, Henderson C E. Neural adhesion molecules L1 and CHL1 are survival factors for motoneurons. J Neurosci Res 2005; 80:593-9.
[32] Villedieu M, Deslandes E, Duval M, Heron J F, Gauduchon P, Poulain L. Acquisition of chemoresistance following discontinuous exposures to cisplatin is associated in ovarian carcinoma cells with progressive alteration of FAK, ERK and p38 activation in response to treatment. Gynecol Oncol 2006; 101:507-19.
[33] Fischer O M, Hart S, Gschwind A, Prenzel N, Ullrich A. Oxidative and osmotic stress signaling in tumor cells is mediated by ADAM proteases and heparin-binding epidermal growth factor. Mol Cell Biol 2004; 24:5172-83.
[34] Primiano T, Baig M, Maliyekkel A, Chang B D, Fellars S, Sadhu J, et al. Identification of potential anticancer drug targets through the selection of growth-inhibitory genetic suppressor elements. Cancer Cell 2003; 4:41-53.
[35] Arlt M J, Novak-Hofer I, Gast D, Gschwend V, Moldenhauer G, Grunberg J, et al. Efficient inhibition of intra-peritoneal tumor growth and dissemination of human ovarian carcinoma cells in nude mice by anti-L1-cell adhesion molecule monoclonal antibody treatment. Cancer Res 2006; 66:936-43.

### EXAMPLE 2

### Abbreviations

ADAM: A Disintegrin And Metalloprotease. BOG: β-octylglycopyranoside. CRABPII: cellular retinoic acid-binding protein II. CTF: C-terminal fragment. ERK: extracellular-signal regulated kinase. hL1wt: human L1 wild type. hL1mutS: human L1 with a mutation of S1248A hL1mutTS: human L1 with mutations of T1247A and S1248A. PAK 1: p21 activated kinase 1. RA: retinoic acid. RAR: retinoic acid receptor. RIP: regulated intramembrane proteolysis. SH3: Src homology 3. TF AP2α: transcription factor activator protein-2.

### Summary

The L1 cell adhesion molecule plays an important role in cell migration, axon growth and guidance in the nervous system. Recent work has also implicated L1 in human carcinoma progression and revealed that L1-expression augmented cell motility, invasion and tumor growth in nude mice, and upregulated proinvasive genes. In the present study we investigated the mechanism of L1 signaling using a cytoplasmic mutant (hL1mutTS) devoid of activity. We demonstrate that the C-terminal fragment of L1 (L1-CTF) is cleaved by ADAM10 and γ-secretase and then translocates to the nucleus. The L1-CTF cooperates with activated ERK to regulate transcription of L1-dependent genes. Antibodies to L1 but also hL1mutTS could suppress ERK-activation, L1-processing and reversed L1-mediated gene regulation. These findings highlight the role of L1 in carcinomas and offer an explanation for the efficacy of L1 antibodies in experimental therapy models for ovarian carcinoma.

### Introduction

L1 cell adhesion molecule (L1-CAM) is a 200-220 kDa transmembrane glycoprotein of the immunoglobulin (Ig) superfamily. It is composed of six Ig-like domains and five fibronectin type III repeats followed by a transmembrane region and a highly conserved cytoplasmic tail (1). L1 is involved in the regulation of cell migration, axon outgrowth and guidance during the development of the nervous system (2-5). Recent studies have shown that the L1 molecule also plays an important role in the ontogeny of human tumors (6-13). In melanoma and ovarian/endometrial carcinoma, L1 expression is associated with poor prognosis (8-10). The mechanism by which L1 contributes to tumor progression has not been clearly established. Meanwhile, antibodies to L1 were shown to have therapeutical potential and can reduce cell proliferation in vitro (11,13), and in vivo growth in a xenograft mouse model for human ovarian carcinoma (14). Thus, L1 might be a novel target for antibody-based therapy against aggressive human tumors. A better understanding of L1 signaling in carcinoma cells and the mode of action of L1 antibodies is therefore urgently needed.

Previous studies have demonstrated that the ectopic expression of L1 can augment tumor growth in NOD/SCID mice (13,15), can enhance cell motility on extracellular matrix proteins (16-18) and invasiveness in matrigel invasion assays (13,19). Interference with L1 expression by genetic manipulation was found to be growth inhibitory in vitro (11). Importantly, a recent study has demonstrated that L1 can induce ERK-dependent gene regulation (18). As revealed by gene chip analysis, the presence of L1 upregulated expression of the motility and invasion related proteins Rac and Rho but also the proteases cathepsin B and L and the β3 integrin subunit (18). Although ERK activation appears to be a crucial element, it remains unclear whether activated ERK alone or only in cooperation with L1 could lead to the expression of these genes.

We demonstrated previously that L1 is cleaved and released from the cell membrane by the metalloprotease ADAM10 (16,20). The soluble L1 ectodomain is also detectable in serum and ascites from ovarian carcinoma patients (9). The involvement of ADAM10 in L1 shedding was recently confirmed in a study using a battery of ADAM-deficient fibroblastic cell lines established from knock-out mice (21). This study showed for the first time that proteolytic cleavage of the extracellular domain of L1 by ADAM10 is followed by intramembrane presenilin-dependent γ-secretase cleavage leading to the generation of a L1 cytoplasmic domain missing the transmembrane region (21). This process, named regulated intramembrane proteolysis (RIP), is an essential step in a variety of signaling pathways. The nuclear translocation of proteins such as Notch, CD44 and the amyloid precursor protein (APP) are known to require ADAM-mediated cleavage (22). All of these proteins are able to translocate to the nucleus after presenilin processing and regulate gene transcription (22). This raised the possibility, that L1-cleavage fragments, in addition to activated ERK, might be required for L1-dependent gene regulation.

In this report, we have analyzed closer the role of L1 cleavage in gene regulation. Using human carcinoma cells as model, we observed that L1 expression indeed led to altered gene expression, enhanced motility, invasiveness in vitro and tumor growth in NOD/SCID mice. Inhibition of RIP via treatment with the γ-secretase inhibitor DAPT blocked L1 proteolytic processing but also L1-dependent gene expression. In addition, activation of the MAPK ERK1/2 seems to be critical for L1-mediated gene regulation. We identified a mutant form of L1 with an alteration of the T1247/S1248 motif in the intracellular domain (hL1mutTS) that failed to activate ERK1/2 and could not serve as substrate for ERK1/2-dependent phosphorylation. Concomitantly, hL1mutTS expressing cells lost the ability to regulate L1-dependent gene transcription and to augment tumor growth. Our results suggest that L1-CTF translocates to the nucleus after processing by ADAMs and γ-secretase and cooperates with activated ERK in transcriptional regulation. Strikingly, L1 antibodies showed similar effects as hL1mutTS. They prevented ERK activation and interfered with L1 processing and, most importantly, were able to reverse the L1-dependent gene expression pattern. These findings provide a rationale for the mode of action of L1 antibodies and suggest that interference with L1 function could become a valuable target for therapy.

### Results

### Functional Role of the Cytoplasmic Part of L1

Previous studies have shown that L1 expression increased cellular motility on ECM components (15-18) and augmented cell invasiveness in matrigel (13,19). Compared HEK293 cells stably transduced with wild-type human L1 (hL1wt) or a deletion mutant missing the cytoplasmic portion of L1 (L1-1247) (23), we initially observed that these effects were lost in the deletion mutant (data not shown). These results suggested that an intact cytoplasmic portion was required for L1-augmented cell motility and invasiveness.

We searched for amino acids within the cytoplasmic portion of L1 responsible for these effects. The cytoplasmic part contains a putative SH3 binding domain with the consensus sequence PINP (Position 1249-1252). The proceeding amino acid S1248 was previously identified as a phosphorylation site for ERK2 (24). Given the known role of ERK in L1-mediated gene regulation (18) and L1-promoted cell motility (17), we altered the ERK-phosphorylation site (S1248A) by site-directed alanine mutagenesis (hL1mutS). A second mutant was constructed including the adjacent threonine (T1247A, S1248A) and was termed hL1mutTS (see FIG. 7A). Both mutants were stably expressed in HEK293 cells. FACS analysis revealed expression of all L1 constructs at the cell surface (FIG. 7B).

Cells expressing hL1mutS showed enhanced motility similar to hL1wt (FIG. 7C). In contrast, cells expressing hL1mutTS lost the ability to augment cell motility and behaved similar to the L1-1247 deletion construct (FIG. 7C). Similar results were observed in matrigel invasion assays (FIG. 7D).

To exclude changes in integrin expression as the mechanism behind motility effects, the adhesion to fibronectin and laminin was examined. No difference in the adhesion to fibronectin between HEK293 cells expressing hL1wt or hL1mutTS was detected (data not shown). There was a slightly reduced adhesion to laminin in HEK293-hL1mutTS cells compared to HEK293-hL1wt cells (data not shown), Analysis of hL1wt and hL1mutTS in stably transfected SW707 cells showed comparable results to HEK293 cells. Thus, using site-directed mutagenesis, we were ably to define amino acids in the cytoplasmic part of L1 that mimicked the effect of the deletion mutant. We concentrated on the characterization of hL1mutTS.

### Analysis of Tumorigenicity in NOD/SCID Mice

The presence of L1 can augment tumor growth in xenotransplanted mice (13,15). We analyzed the effects of hL1mutTS on tumor growth in NOD/SCID mice. To allow side by side comparison, we injected HBEK293 into the right flank and HEK293-hL1wt into the left flank of mice. Likewise, HEK293-hL1wt cells were injected into the right flanks and HEK293-hL1mutTS cells were injected into the left flanks of mice. As expected, we observed significantly augmented growth of HEK293-hL1wt tumors in comparison to untransfected HEK293 tumors (FIG. 7E). Strikingly, HEK293-hL1mutTS cells did not enhance tumor growth in NOD/SCID mice (FIG. 7E).

An important parameter of tumor growth is cell proliferation. It was previously shown that L1 positive cells proliferate to a greater extent than L1 negative cells under low serum conditions (13,18). We observed that hL1mutTS compared to hL1wt decreased cell proliferation under low (0.5% FCS) serum conditions. This effect was prominent after 48 hr of culture (data not shown).

We verified these results in a second model system composed of SW707 colon carcinoma cells. SW707-hL1wt cells augmented tumor growth in vivo in agreement with previous results (13). Cells expressing hL1mutTS showed similar in vivo growth as mock-transfected SW707 cells. We concluded that the T1247/S1248 motif in the CTF of hL1 has a significant impact on tumor growth in vivo.

Further Characterization of hL1mutTS

Biochemical analysis confirmed the presence of full-length L1-220 and the cleavage fragments L1-85, L1-42 and L1-32 in both hL1wt and hL1mutTS expressing cells (FIG. 8A). The release of soluble L1-200 into the supernatant was comparable either under constitutive or PMA-induced conditions (FIG. 8B).

Soluble L1 is able to stimulate cell migration (16,21). Indeed, a recombinant L1-Fc protein enhanced cell migration of untransfected HEK293 cells (four-fold increase) and weakly augmented cell migration of hL1wt expressing cells (FIG. 8C). In contrast, cells expressing hL1mutTS showed no L1-Fc stimulated migration (FIG. 8C).

We proposed previously that soluble L1, released by cells, could drive migration by an autocrine/paracrine loop (9,16). Since hL1wt and hL1mutTS were cleaved from the membrane and released into the supernatant to a similar extent (FIG. 8B), we investigated whether soluble hL1mutTS was also functional. We analyzed the ability to stimulate HEK293 cell migration. There was no difference between soluble hL1wt or hL1mutTS forms (FIG. 8D). These results suggest that the failure of mutant L1 to promote migration was not due to a defect in shedding or a non-active soluble form.

### Analysis of L1-Dependent Signaling

Some L1 functions have been shown to involve ERK1/2 activation (17,24). Indeed, a recent study has demonstrated that L1 expression causes sustained ERK activation, leading to enhanced motility of cells and augmented the activity of ERK1/2-dependent genes (18). We observed that under serum conditions, HEK293 and HEK293-hL1wt cells showed constitutive phosphorylation of ERK (FIG. 8E). In contrast, in hL1mutTS expressing cells no phosphorylation of ERK was observed (FIG. 8F).

Several pathways can cause ERK phosphorylation. Therefore, we examined possible mechanisms for the L1-dependent ERK phosphorylation. We proposed before that soluble L1 binds to integrins and therefore stimulates cell migration. Activated integrins can phosphorylate the focal adhesion kinase (FAK). Indeed, we could show that FAK was phosphorylated with no difference in hL1wt or hL1mutTS expressing cells (FIG. 8E). L1 negative cells showed lower FAK activation. PAK 1, a downstream target of FAK, was also activated in hL1wt and hL1mutTS expressing cells (FIG. 8E). This suggested that signaling by integrins is not altered in HEK293-hL1mutTS cells.

Thelen et al. (17) could demonstrate that Src plays a crucial role in L1-mediated cell migration. Dominant-negative Src suppressed L1-mediated cell motility. Therefore, we analyzed the phosphorylation status of Src and observed enhanced activation in HEK293-hL1wt cells compared to HEK293-hL1mutTS and HEK293 cells (FIG. 8E). These results indicate that L1 mediates activation of Src. We concluded that L1-dependent activation of ERK involves Src and/or other molecules in HEK293-hL1wt cells. This pathway is blocked in cells expressing hL1mutTS.

The amino acid S1248, that is mutated in hL1mutTS, comprises an ERK2 phosphorylation site (24). To confirm that ERK2 could indeed not phoshorylate L1 in this position, we made use of GST-fusion proteins encoding the cytoplasmic part of hL1wt and hL1mutTS. Recombinant Src-kinase could readily phosphorylate both GST-fusion proteins whereas ERK2 could only phosphorylate the GST-hL1wt construct (FIG. 8F).

Taken together, these results showed that hL1mutTS cannot activate ERK in a constitutive manner. At the same time, hL1mutTS cannot serve as acceptor for ERK2 phosphorylation.

### Mutant L1 Acts in a Dominant Negative Fashion

Next, we examined whether hL1mutTS overexpression could suppress cell motility of wild type L1 expressing cells. Therefore, we transfected transiently HEK293-hL1wt or HEK293 cells with hL1mutTS. Overexpression of hL1mutTS inhibited the migration of HEK293-hL1wt but not HEK293 cells (FIG. 9A). Inhibition of cell migration was also observed with a dominant-negative form of ADAM10 (ADAM10-DN) and in the presence of the MEK inhibitor PD59098. Transfection with the empty vector did not affect cell migration. Overexpression of ADAM10 enhanced cell motility (data not shown).

To allow efficient expression of hL1mutTS in carcinoma cells, we constructed a recombinant adenovirus. Transduction of L1-negative KS breast carcinoma cells led to a dose-dependent expression of hL1mutTS (data not shown). We next transduced KS and the L1 positive cell lines OVMz, SKOV3ip and M068 with hL1wt or hL1mutTS encoding adenoviruses and analyzed the effect on cell migration. In agreement with previous results (15), transduction with hL1wt enhanced migration, whereas hL1mutTS decreased the migration only of L1 positive cells (FIG. 9B). YFP-adenovirus used as control did not affect cell motility.

The hL1mutTS-adeno also strongly suppressed the matrigel invasion of OVMz and SKOV3ip cells (FIG. 9C). Thus, we concluded that hL1mutTS possesses a dominant-negative activity which is specific towards L1 expressing cells.

To find out whether the dominant-negative function of the hL1mutTS could interfere with ERK activation, we transduced OVMz cells with the hL1mutTS encoding adenovirus. This transduction clearly suppressed ERK phosphorylation (FIG. 9D), whereas ADAM10 or ADAM10-DN adenoviruses were without effect (FIG. 9D). We concluded that ERK activation was impaired due to the dominant-negative form of L1 in carcinoma cells expressing endogenous L1, whereas modification of L1 cleavage by overexpression of ADAM10 does not influence ERK phosphorylation under the experimental conditions.

hL1mutTS Alters Gene Expression in HEK293 Cells

Sustained ERK activation leads to nuclear translocation of ERK and the induction of ERK-dependent genes (25,26). We employed gene chip analysis to investigate differential gene expression in HEK293, HEK293-hL1wt and EK293-hL1mutTS cells. Differential hybridization revealed that of the 1920 cDNAs on the chip, appr. 448 were up- or downregulated more than onefold in HEK293-hL1wt cells (data not shown). We concentrated our analysis on genes important for invasion, motility and tumor growth regulation. Several identified genes were confirmed by qPCR for differential expression at the mRNA level. In HEK293-hL1wt cells, we observed upregulation of genes such as cathepsin B, β3 integrin and the transcription factors HOX A9, AP2α. The apoptosis related gene Mdm 2 was also upregulated whereas downregulation was noticed for the retinoic acid (RA) binding protein CRABPII, and the apoptosis-inducing genes STK 39 and IER 3 (FIG. 10A). The same set of genes remained unchanged in hL1mutTS expressing cells and was similar to non-transfected cells (FIG. 10A). Similar observations were made in the colon carcinoma cell line SW707.

The expression of CRABPII, that is essential for the nuclear transport of RA and tumor growth suppression (27), was dramatically reduced. We observed CRABPII downregulation in hL1wt cells compared to parental or hL1mutTS cells (FIG. 10A). The qPCR results for cathepsin B and CRABPII were confirmed by Western blot (FIG. 10B) and for cathepsin B and β3-integrin by FACS analysis (FIG. 10C).

CRABPII channels RA to the nucleus. There, RA binds to its specific receptor RAR and regulates gene expression of RAR elements leading to a decrease in cell proliferation. Therefore, we treated cells with RA and then determined the level of cell proliferation. As expected from the expression analysis, hL1wt expressing cells were more resistant to RA-mediated growth inhibition than hL1mutTS expressing cells (FIG. 10D). Similar results were obtained in SW707 cells. Thus, our results demonstrated that L1 expression causes changes in gene expression leading to altered properties of carcinoma cells. The T1247/S1248 site in L1 is essential for this gene regulation.

### Metalloprotease and Presenilin Cleavage are Essential for L1-Mediated Gene Regulation

A recent publication has demonstrated that L1 is processed by γ-secretase following initial ectodomain cleavage by ADAM10 (21). Consecutive cleavage by both enzymes is a hallmark of Notch, APP and CD44 signaling that is followed by translocation of the intracellular portion to the nucleus (22). To verify this observation for tumor cells, we analyzed the processing of L1-32 in more detail. CHO-hL1wt cells were cultivated for 48 h in the presence of the presenilin inhibitor IX (DAPT). Microsomal membranes were then isolated and assayed for in vitro γ-secretase activity as described (28). For this, membranes were incubated for 2 h at 37° C. and then the membranous and soluble fractions were separated by ultracentrifugation. Presenilin cleavage is expected to release the L1-28 from the membrane into the supernatant (21). Indeed, we detected L1-28 in the supernatant fraction (FIG. 11A, lane 2). Cells preincubated with the presenilin inhibitor could not release this fragment into the supernatant. In these pretreated cells we could detect the L1-32 in the membrane pellet fraction (FIG. 1A, lane 3). These findings demonstrate that L1 undergoes ADAM10 and presenilin cleavage.

In cell lysate, we noticed that L1-28 was difficult to detect most likely due to rapid degradation and/or low abundancy. The treatment of cells with DAPT lead to a strong increase in the metalloprotease cleavage fragment L1-32 (FIG. 11B and see below). This was not due to enhanced L1 cleavage by ADAM10 but rather the effected of blocked L1-32 processing as the amount of soluble L1 in the medium was not increased by the treatment (FIG. 11B). In agreement with this notion, accumulation of L1-32 by DAPT was also seen in the presence of the metalloproteinase inhibitor TAPI-0 (data not shown).

We next investigated whether L1-mediated gene regulation was dependent on L1-processing, by carrying out inhibition experiments. We preincubated HEK293 and HEK293-hL1wt cells for 96 h with DAPT, TAPI-0 or both inhibitors together. QPCR analysis showed that the L1-dependent regulation of transcription for cathepsin B and CRABPII was blocked by TAPI-0 or DAPT or both (FIG. 11C). Importantly, both compounds did not interfere with ERK activation in treated cells (FIG. 11D). These results clearly show that metalloprotease and presenilin-mediated cleavage of L1, independent of ERK activation, play an essential role L1-mediated gene regulation.

### The C-Terminal Fragment of L1 Translocates to the Nucleus

To further demonstrate that the L1-cleavage fragments were involved in transcriptional regulation, we performed ChIP assays on chromatin samples from HEK293, HEK293-hL1wt and HEK293-hL1mutTS cells. As prototype genes regulated by L1, we chose to analyze the cathepsin B, CRABPII and β3-integrin promoters. To rule out non-specific effect, the β-actin promoter was used as negative control. Occupancy of the promoter was tested after chromatin-IP with pcytL1 followed by PCR analysis of the associated DNA extractions with primers specific for the respective promoters. As shown in FIG. 12A, specific bands of the expected size were only noted in hL1wt expressing cells. No signal was obtained using β-actin primers. These findings indicate that the hL1wt-CTF but not the hL1mutTS-CTF can interact with the cathepsin B, β3 integrin and CRABPII promoters and in doing so may regulate transcription.

Confocal microcopy of CHO-hL1wt cells with pcytL1 was used to confirm the presence of L1-CTF in the nucleus. As depicted in FIG. 12B, staining was detected within the cytoplasm and the nucleus of CHO-hL1wt but not in CHO cells. Strikingly, CHO-hL1mutTS cells showed a different staining pattern. Staining was mostly present in the cytoplasm and at the nuclear membrane but was less pronounced in the nucleus (FIG. 12B).

We next investigated the presence of L1-CTF in the nucleus by biochemical means. Nuclei from HEK293-hL1wt, HEK293-hL1mutTS and HEK293 cells were purified using a well established protocol (29). The purity of the nuclear fractions was examined by Western blot using marker proteins. A representative purification is shown in FIG. 12C. We observed that the isolated nuclei were not contaminated by cytosolic proteins (marker moesin) or ER specific proteins (BiP/GRP78 marker). Nucleoporin (nuclear marker) was exclusively present in the nuclear fraction.

Using again the pcytL1 antibody to the cytoplasmic portion of L1, we detected two C-terminal fragments with sizes of appr. 32 kDa and 28 kDa, respectively, in the nuclear fractions (FIG. 12D) of serum grown cells. In contrast, in hL1mutTS expressing cells and under serum-free conditions only L1-32 was present. In L1-negative cells, no signal was detected (FIG. 12D). We concluded from these results that L1 processing was dependent on serum factors. Most importantly, hL1mutTS was not processed to L1-28 and therefore most likely cannot act in transcriptional regulation.

Antibodies to L1 can Reverse L1-Dependent Gene Regulation by Interfering with L1 Signaling

Antibodies to L1 were shown to prevent tumor cell proliferation in vitro (II) and tumor growth in vivo in a xenograft mouse model for human ovarian carcinoma (14). We investigated whether the suppressive effect of L1-antibodies might be mechanistically similar to the effect observed here for L1mutTS.

We examined in more detail the mode of action of L1 antibodies using mAb L1-11A, an antibody that was found to be effective in vivo (14). Strikingly, mAb L1-I 1A efficiently inhibited the serum-induced activation of ERK in SKOV3ip cells in vitro (FIG. 13A). There was no inhibition seen with isotype matched control antibodies or the control mAb 38-12. Analysis of cell lysate from antibody treated cells also revealed an increase in L1-32 but no increase in soluble L1-200 (FIG. 13B). This effect was also seen in the presence of TAPI-0 suggesting that the antibody did not activate shedding per se (FIG. 13B). These findings matched the results obtained with the presinilin inhibitor DAPT (see FIG. 11B).

We next examined whether mAb L1-11A could affect the gene expression profile of SKOV3ipcells. Indeed, qRT-PCR analysis of cells treated with mAb L1-11A antibody versus control antibody showed significant changes in expression of prototype L1-regulated genes such as HOX A9, β3-integrin, IER 3 and STK 39 (FIG. 13C).

To analyze whether the observed effects were unique for the epitope recognized by mAb L1-11A, we produced additional antibodies to L1. The novel antibody was specific for L1 as confirmed by FACS analysis on SKOV3ip cells and Western blot analysis on tumor cell lysates (FIGS. 8A and B). As shown in FIG. 7A, mAb L1-14.10 could efficiently inhibit ERK activation in the presence of serum factors. Also, both mAbs to L1 blocked the invasion of SKOV3ip cells in matrigel (FIG. 14C).

Finally, the novel antibody L1-14.10 was tested in comparison to mAb L1-11A and control antibodies to EpCAM (HEA125) for the inhibition of tumor growth in nude mice. The novel mAb L1-14.10 was equal in suppressing tumor growth in vivo compared to L1-11A, whereas mAb HEA125 had no effect on tumor growth (FIG. 14C).

### Discussion

L1 is a type 1 transmembrane protein that is expressed by human carcinomas and melanomas and has been linked to poor prognosis in several studies (8-10,12). L1 undergoes regulated proteolysis that takes place at the cell surface and in released exosomes and involves the metalloprotease ADAM10 (16,20,30). Recent studies have shown that L1 is also cleaved by the γ-secretase complex (21). Here we provide evidence that the process of regulated proteolysis is important for L1-dependent signaling in human tumors. We demonstrate that i) the proteolytically processed cytoplasmic fragments of L1 are present in the nucleus; ii) the expression of hL1wt augments cell motility, invasiveness and tumor growth; iii) the presence of hL1wt causes sustained ERK activation and augments transcriptional activity of proinvasive genes; iv) hL1mutTS carrying a mutation in the cytoplasmic portion of amino acids T1247A/S1248A abolishes all effects seen with hL1wt; v) Gene regulation by L1 is suppressed in cells treated with specific inhibitors for ADAMs and presenilins; vi) antibodies to L1 mimicked the effects of hL1mutTS by suppressing ERK activation, inhibiting L1-32 processing and reverting the L1-induced transcriptional program. These results strongly suggest that ERK activation and L1-CTF nuclear translocation are required for L1-dependent signaling that can be targeted with L1 antibodies.

Recently, Maretzky et al. (21) demonstrated that L1-mediated neural cell migration was influenced by RIP of L1. The authors showed that RIP takes place after initial metalloprotease cleavage by ADAM10. This study in mouse fibroblastic cell lines did not investigate nuclear translocation and gene regulation. In our study, we detected L1-32, the initial ADAM10 cleavage fragment, in cell lysate, microsomal membranes and in the nucleus of L1-expressing cells. We observed that L1-32 was converted into a soluble fragment of appr. 28 kDa. This step could be blocked in the presence of a presenilin inhibitor. These results are consistent with the data obtained in mouse embryonic fibroblasts (21) and demonstrate for the first time that a similar processing of L1 is operating in human carcinoma cells.

Our study also shows that the expression of L1 lead to global changes in gene expression profiles. In more detail, we observed that transcription factors such as AP2α and HOX A9 were upregulated in hL1wt expressing cells. Based on their role in gene regulation, these transcription factors could perform a relevant role in tumor progression (31). In agreement with Silletti et al. (18), we could also show that proinvasive proteins such as the cysteine protease cathepsin B and the motility associated β3 integrin were upregulated in hL1wt expressing cells. Furthermore, the cellular retinoic acid binding protein CRABPII, that is known to be a tumor suppressor, was significantly downregulated in hL1wt expressing cells. CRABPII channels RA to RAR, thereby enhancing the transcriptional activity of the receptor. RA can suppress cell proliferation and transcription (32). Collectively, our gene expression analysis, together with that of others (18), indicates that global changes induced by hL1wt could favor a more tumorigenic and invasive phenotype in carcinoma cells.

As demonstrated in our study for cathepsin B and CRABPII, L1-mediated gene regulation was dependent on ADAM and presenilin processing as it was blocked in the presence of the respective inhibitors. Chromatin-IP demonstrated that the L1-CTF was associated with promoter regions of the cathepsin B, β3 integrin and CRABPII genes but not with β-actin promoter. This clearly established a link between L1-CTF nuclear translocation and L1-mediated gene regulation.

Of great value for our study was the discovery of a cytoplasmic mutant of L1 that abrogated L1 mediated effects. In all functional aspects (motility, invasiveness, gene regulation and tumor growth), hL1mutTS expressing cells behaved like cells that express no L1. This suggested that the point mutations had rendered the L1 protein functionally inactive. How does hL1mutTS cause these effects? Our results suggest at least two mechanisms by which hL1mutTS causes these effects: (i) the mutant protein efficiently suppressed L1-mediated ERK activation and (ii) it abolished the function of the L1-CTF to serve as a transcription factor.

We observed, in hL1mutTS expressing cells that the ERK1/2 phosphorylation was strongly diminished (FIG. 8E). The hL1mutTS-adenovirus could act in a dominant-negative fashion by suppressing ERK activation in L1 expressing carcinoma cells (FIG. 9). It remains to be investigated how hL1mutTS mediates this effect.

ERK1/2 are serine-threonine kinases which can phosphorylate many proteins including transcription factors, cytoskeletal proteins, membrane proteins and other kinases (33). ERK1/2 activation can be distinguished into either transient or sustained modes. The latter mode is required for the translocation of activated ERK1/2 from the cytoplasm to the nucleus where it can regulate gene transcription (25,26,33). Recent reports have demonstrated a close association between L1 and sustained ERK1/2 activation in carcinoma cells (13,18). Recombinant ERK2 could phosphorylate S1248 and S1204 in the cytoplasmic domain of L1 (24) and both sites were phosphorylated in postnatal rat brain (34). Indeed, we also found that a GST-fusion protein comprising the hL1mutTS cytoplasmic part could not be phosphorylated by recombinant ERK as S1248 is missing (FIG. 8F). This suggested that hL1mutTS is defective in ERK1/2 activation but also cannot serve as a substrate for ERK.

Why is hL1mutTS defective in ERK activation? Thelen et al. (17) could show that Src is required for enhanced cell motility of L1 expressing cells. Expression of a dominant-negative Src (K295M) mutant in L1-transfected HEK293 cells decreased L1-potentiated migration to the level of untransfected cells. C-Src is required for endocytosis of L1 (24,35), the regulation of neurite outgrowth on L1 coated surfaces (36) and L1-induced ERK activation (17,24,33). In our study we observed higher Src activation in hL1wt cells compared to L1-negative cells and this activation was decreased in hL1mutTS cells. Therefore, the mutated amino acid motif in the cytoplasmic portion of L1 is involved in Src activation. ERK1/2 is a downstream target of Src. Our data suggest that the loss of the T1247/S1248 motif prevented Src-dependent ERK1/2 activation. Another possibility is that the interaction with RanBPM is effected in the hL1mutTS expressing cells. RanBPM is a novel L1-interacting protein that acts as an adaptor protein linking L1 to the ERK pathway (37). It remains to be investigated whether hL1mutTS has lost the ability to bind efficiently to RanBPM.

The ability of L1 to support sustained ERK activation was previously found to be critically dependent upon cell-cell contact and the presence of serum (13,18). In this context, it is interesting to note that in HEK293-hL1wt cells the nuclear L1-28 fragment was only detectable when cells were grown under serum conditions. In contrast, in hL1mutTS expressing cells only the L1-32 fragment was present and L1-28 was not detected even under serum conditions. Moreover, the Chromatin IP with pcytL1, an antibody to the C-terminus of L1, suggested that only in HEK293-L1wt cells the L1-CTF was able to join a transcriptional complex. This raises the possibility that hL1mutTS was not properly processed and therefore was functionally inactive. Indeed, recent studies have shown that γ-secretase activity is under control of the MAP-kinase pathway (38). For L1 signaling, further experiments are needed to investigate why hL1mutTS is not transcriptionally active.

We observed that mAbs to L1 efficiently mimicked the effects seen with hL1mutTS. Antibodies to L1 could drastically reduce ERK phosphorylation and this was observed for all L1 antibodies tested suggesting that there was no dependency on a particular L1-epitope. It is interesting to note that in neuronal cells, L1-crosslinking with antibodies lead to ERK activation (24,39) whereas in carcinoma cells just the opposite is observed. The presence of antibodies to L1, similar to the presinilin inhibitor DAPT, caused an accumulation of L1-32 that was not due to enhanced cleavage as it was not associated with increased levels of soluble L1 (FIG. 13B). It is possible that the crosslinking effect of the antibody at the cell membrane rendered the L1-32 cleavage fragment inaccessible to further presenilin processing. The effects on L1 processing by the mAb were accompanied by changes in the expression pattern of L1 regulated genes. Thus, for the first time a picture is emerging that offers an explanation for the efficacy of L1 antibodies in experimental therapy models for ovarian carcinoma.

Based on our results, we propose the following model: L1 undergoes sequential cleavage by ADAM10 and presenilin and both proteolytic products can be detected in the nucleus. Concomitantly, L1 promotes sustained ERK activation leading to nuclear translocation of ERK1/2. L1-CTF is phosphorylated by activated ERK2 and can join a transcriptional complex that in our example was found to associate with several promoter sites. The hL1mutTS and L1 antibodies reduce sustained activation of ERK and prevent L1-dependent gene regulation. This offers the possibility to target L1 in positive human carcinomas. The inactivation of L1 might be beneficial for blocking the growth and dissemination of tumors.

### Materials and Methods

### Cells and DNAs

The ovarian tumor cell lines OVMz, SKOV3ip, the breast cancer cell line KS and SW707 colon carcinoma cells were described before (13,20). The primary ovarian carcinoma cell line M068 was obtained from Dr. Ingrid Herr (DKFZ, Heidelberg). The human epithelial kidney cell line HEK293, chinese hamster ovary (CHO) cells and SW707 cells stably expressing human L1 (hL1wt) and mutant L1 (hL1mutS, hL1mutTS) were established by transfection with Superfect (Stratagene, Heidelberg, Germany). All cells were cultivated in DMEM supplemented with 10% FCS at 37° C., 5% CO₂ and 100% humidity. L1 mutagenesis was performed with the QuikChange™ Site-Directed Mutagenesis Kit essentially as described by the manufacturer (Stratagene, Heidelberg, Germany). All constructs were verified by sequencing.

### Adenovirus Production

Recombinant adenovirus was produced as described before (15). YFP-TM adenovirus was a kind gift of Dr. P. Keller (MPI for Cell Biology, Dresden).

### Chemicals and Antibodies

Antibodies to the ectodomain (L1-11A, subclone of mAb UJ 127.11) or cytoplasmic domain (pcyt-L1) of human L1 were described (16). The mAb HEA-125 to EpCAM was previously described (40). Novel mAb to L1 (mAb L1-14.10) was obtained after immunization of mice with human L1-Fc protein comprising the ectodomain of L1 as described (41). Antibodies to ERK1, phospho-ERK1/2, FAK and phospho-FAK (p125) were purchased from BD-Transduction (Heidelberg, Germany). The Antibody to phospho-PAK 1 was purchased from Cell Signaling (New England Biolabs, Frankfurt, Germany) and antibodies to Src and Phospho-Src were purchased from Abeam (Biozol Diagnostica, Eching, Germany). The antibody against cathepsin B was from Zymed (Invitrogen, Karlsruhe, Germany) and the antibody to CRABPII was from Santa Cruz (Santa Cruz, Heidelberg, Germany). Secondary antibodies were obtained from Dianova (Dianova, Hamburg, Germany). Antibodies to nucleoporin and BiP/GRP78 were from the organelle kit (BD-Transduction, Heidelberg, Germany). Retinoic acid was obtained from Sigma. The MEK inhibitor PD59098 was obtained from Calbiochem (Bad Soden, Germany). The human L1-Fc protein has been described (16).

### Analysis of L1 Shedding

Assays were carried out as described previously (42). Briefly, cell monolayers in serum-free medium were stimulated at 37° C. with or without PMA (50 ng/ml). Supernatants were collected and the cells were removed from the tissue culture plastic surface by treatment with PBS/5 mM EDTA. Cell pellets were lysed in lysis buffer (20 mM Tris/HCl pH 8.0 containing 1% β-octylglycopyranoside (BOG), 150 mM NaCl, 1 mM PMSF), cleared by centrifugation and mixed with two-fold concentrated reducing SDS-sample buffer. The detection of soluble L1 in the supernatant by L1-specific capture ELISA has been described before (Mechterheimer et al, 2001).

### DNA Chip Analysis and Quantitative PCR

mRNA was isolated using the Quiagen RNAeasy mini kit (Quiagen Hilden, Germany). The cDNA array contained 1540 DNA fragments of oncological relevance and 60 control genes (http://www.rzpd.de/products/microarrays/oncochip.shtml). After exposing of the hybridized membranes, the PhosphorImager screens were scanned (Fuji FLA-3000, 100 μm resolution, Fuji BAS-reader software). The primary image analysis (estimation of nVol grey level values for each individual spot) was performed using the ArrayVision software package (Interfocus), which had been adjusted to the 5×5 array before. The background was corrected locally in each 5×5 field by subtracting the empty spot signal (average signal of 3 spots, see above). Normalization was performed via the average signal intensity (without empty spots) on the whole membrane. Two independent hybridizations were performed for each experiment. For qPCR the cDNA was purified on Microspin G-50 columns (Amersham Biosciences, Freiburg, Germany) and quantitated by Nanoprop spectrophotometer (ND-1000, Kisker-Biotechnology, Steinfurt, Germany). Primers for qPCR were designed with the DNA Star Program and were produced by MWG (Ebersberg, Germany). β-actin was used as an internal standard. The PCR reaction was performed with the SYBRgreen mastermix (Applied Biosystems, Darmstadt, Germany). The sequence of primers used is available on request.

### GST Fusion Proteins

Fusion proteins comprising the cytoplasmic portion of hL1wt and hL1mutTS (beginning with F1142) were constructed using conventional techniques. For kinase reactions, 2 μg of purified fusion protein was labelled using ³²P-labelled γ-ATP and recombinant SRC (Biomol, Hamburg, Germany) or recombinant ERK2 (Calbiochem). The reactions were carried out as suggested by the manufacturers.

### Biochemical Analysis

SDS-PAGE under reducing conditions and transfer of separated proteins to Immobilon membranes using semi-dry blotting were described before (42). After blocking with 5% skim milk in TBS, the blots were incubated with the respective primary antibody followed by peroxidase conjugated secondary antibody and ECL detection. Chromatin IP assays were done essentially as described (43).

### Immunofluorescence Staining and FACS Analysis

The staining of cells with mAbs and PE-conjugated secondary antibodies has been described (16). Cells were analysed with a FACScan using Cellquest software (Becton & Dickinson. Heidelberg, Germany).

### Transmigration and Matrigel Invasion Assays

This assay has been described before (16). Briefly, ECM proteins or BSA for control were coated on the backside of Transwell chambers (Costar, 6.5 mm diameter, 5 μm pore size). Cells in RPMI 1640 medium containing 0.5% BSA were seeded into the upper chamber and allowed to transmigrate to the lower compartment. Transmigrated cells, adherent to the bottom of the membrane, were stained with crystal violet solution. Cell associated dye was eluted with 10% acetic acid and the OD was determined at 595 nm using a plate reader. Tumor cell invasion in vitro was determined in a double-filter assay as described (44). Each experiment was done in quadruplicate and the mean values±SD are presented.

### Adhesion Assay and Cell Proliferation

96-well plates were coated overnight with ECM substrates (fibronectin, laminin or vitronectin) or BSA for control. After blocking with BSA, 1×10⁵ cells were filled into the chambers and allowed to bind. Unbound cells were removed with 80% Percoll and adherent cells were fixed with glutardialdehyde in 90% Percoll. Fixed cells were stained with crystal violet and then extensively washed with ddH₂O. The dye was eluted in 10% acetic acid and OD was measured at 595 nm using an ELISA plate reader. Each experiment was performed in triplicate and the mean values±SD are presented. Cell proliferation under low serum was measured by Coulter Counter after 24, 48 and 72 hr.

### γ-Secretase Cleavage Assay

The assay was carried out as described (28).

### Nucleus Purification

Nuclei purification was done as described (29). Briefly, adherent cells (10⁷) were trypsinized and washed twice with PBS and buffer A (10 mM Tris-HCl, pH 7.4, 8.3 mM KCl, 1.5 mM MgSO₄, 1.3 mM NaCl). The cells were resuspended in buffer A and swollen for 30 min on ice. After centrifugation, cells were resuspended in buffer B (Buffer A supplemented with 0.5% NP-40 and 1 mM PMSF). Nuclei and cytosol were prepared by passing the suspension through a 23-gauge needle followed by 20 dounces in a homogenizer. Crude nuclei were sedimented (10 min, 1000×g), resuspended in buffer C (buffer A containing 1 mM PMSF), sedimented again and resuspended in buffer S1 (0.25M sucrose, 1.5 mM MgSO₄, 1 mM PMSF). The suspension was underlayered with buffer S2 (0.88 M sucrose, 0.05 mM MgSO₄) and centrifuged (15 min, 2500×g). The sediment containing the purified nuclei were resuspended in 60 oil of buffer S3 (0.34 M sucrose, 0.05 mM MgSO₄, 1 mM PMSF), sonicated briefly and prepared for SDS-PAGE.

### In Vivo Experiments

6-week-old NOD/SCID female mice (4 animals per group) were injected s.c. with 10⁷ cells stably expressing hL1wt or hL1mutTS. For control, untransfected HEK293 or mock-transfected SW707 cells were used. One type of cells was injected into the right flank and the other type was injected into the left flank of the same animal. Tumor growth was monitored for the indicated length of time at which point the experiment was terminated and tumors were collected for histological evaluation. At different time points the tumor was measured and the volume was calculated using the formula: V=(L×W²)π/6. All experiments were carried out under the German animal protection law and were approved by local authorities.

### Statistical Analysis

For the analysis of statistical significance the Student's t test was used.

### REFERENCES

1. Moos, M., Tacke, R., Scherer, H., Teplow, D., Fruh, K., and Schachner, M. 1988. Neural adhesion molecule L1 as a member of the immunoglobulin superfamily with binding domains similar to fibronectin. Nature, 334: 701-703.
2. Kamiguchi, H. 2003. The mechanism of axon growth: what we have learned from the cell adhesion molecule L1. Mol. Neurobiol. 28: 219-228.
3. Kenwrick, S., Watkin,s A., and DeAngelo, E. 2000. Neural cell recognition molecule L1: relating biological complexity to human disease mutations. Hum. Mol. Genet. 9: 879-886.
4. Hortsch, M. 2000. Structural and functional evolution of the L1 family: are four adhesion molecules better than one? Mol. Cell. Neurosci. 15: 1-10.
5. Schachner, M. 1997. Neural recognition molecules and synaptic plasticity. Curr Opin Cell Biol, 9: 627-634.
6. Katayama, M., Iwamatsu, A., Masutani, H., Furuke, K., Takeda, K., Wada, H., Masuda, T., and Ishii, K. 1997. Expression of neural cell adhesion molecule L1 in human lung cancer cell lines. Cell Struct. Funct. 22: 511-516.
7. Senner, V., Kismann, E., Puttmann, S., Hoess, N., Baur, I., and Paulus, W. 2002. L1 expressed by glioma cells promotes adhesion but not migration. Glia 38: 146-154.
8. Thies, A., Schachner, M., Moll, I., Berger, J., Schulze, H. J., Brunner, G, and Schumacher, U. 2002. Overexpression of the cell adhesion molecule L1 is associated with metastasis in cutaneous malignant melanoma. Eur. J. Cancer 38: 1708-1716.
9. Fogel, M., Gutwein, P., Mechtersheimer, S., Riedle, S., Stoeck, A., Smirnov, A., Edler, L., Ben-Arie, A., Huszar, M., and Altevogt, P. 2003. L1 expression as a predictor of progression and survival in patients with uterine and ovarian carcinomas. Lancet 362: 869-875.
10. Fogel, M., Mechtersheimer, S., Huszar, M., Smimov, A., Abu, D. A., Tilgen, W., Reichrath, J., Georg, T., Altevogt, P., and Gutwein, P. 2003. L1 adhesion molecule (CD 171) in development and progression of human malignant melanoma. Cancer Lett. 189: 237-247.
11. Primiano, T., Baig, M., Maliyekkel, A., Chang, B. D., Fellars, S., Sadhu, J., Axenovich, S. A., Holzmayer, T. A. and Roninson, I. B. 2003. Identification of potential anticancer drug targets through the selection of growth-inhibitory genetic suppressor elements. Cancer Cell 4: 41-53.
12, Allory, Y., Matsuoka, Y, Bazille, C., Christensen, E. I., Ronco, P., and Debiec, H. 2005. The L1 cell adhesion molecule is induced in renal cancer cells and correlates with metastasis in clear cell carcinomas. Clin Cancer Res. 11: 1190-1197.
13. Gavert, N., Conacci-Sorrell, M., Gast, D., Schneider, A., Altevogt, P., Brabletz, T., and Ben-Ze'ev, A. 2005. L1, a novel target of beta-catenin signaling, transforms cells and is expressed at the invasive front of colon cancers. J. Cell Biol. 168: 633-642.
14. Arlt, M. J., Novak-Hofer, I., Gast, D., Gschwend, V., Moldenhauer, G., Grunberg, J., Honer, M., Schubiger, P. A., Altevogt, P. and Kruger, A. 2006. Efficient inhibition of intra-peritoneal tumor growth and dissemination of human ovarian carcinoma cells in nude mice by anti-L1-cell adhesion molecule monoclonal antibody treatment. Cancer Res. 66: 936-943.
15. Gast, D., Riedle, S., Schabath, H., Schlich, S., Schneider, A., Issa, Y., Stoeck, A., Fogel, M., Joumaa, S., Wenger, T., Herr, I., Gutwein, P., and Altevogt, P. 2005. L1 augments cell migration and tumor growth but not beta3 integrin expression in ovarian carcinomas. Int. J. Cancer 115: 658-665.
16. Mechtersheimer, S., Gutwein, P., Agmon-Levin, N., Stoeck, A., Oleszewski, M., Riedle, S., Postina, R., Fahrenholz, F., Fogel, M., Lemmon, V., and Altevogt, P. 2001. Ectodomain shedding of L1 adhesion molecule promotes cell migration by autocrine binding to integrins. J. Cell Biol. 155: 661-674.
17. Thelen, K., Kedar, V., Panicker, A. K., Schmid, R. S., Midkiff, B. R., and Maness, P. F. 2002. The neural cell adhesion molecule L1 potentiates integrin-dependent cell migration to extracellular matrix proteins. J. Neurosci. 22: 4918-4931.
18. Silletti, S., Yebra, M., Perez, B., Cirulli, V., McMahon, M., and Montgomery, A. M. 2004. Extracellular signal-regulated kinase (ERK)-dependent gene expression contributes to L1 cell adhesion molecule-dependent motility and invasion. J. Biol. Chem. 279: 28880-28888.
19. Meier, F., Busch, S., Gast, D., Goppert, A., Altevogt, P., Maczey, E., Riedle, S., Garbe, C. and Schittek, B. 2006. The adhesion molecule L1 (CD171) promotes melanoma progression. Int. J. Cancer 119: 549-555.
20. Gutwein, P., Mechtersheimer, S., Riedle, S., Stoeck, A., Gast, D., Joumaa, S., Zentgraf. H., Fogel, M., and Altevogt, P. 2003. ADAM10-mediated cleavage of L1 adhesion molecule at the cell surface and in released membrane vesicles. FASEB J. 17: 292-294.
21. Maretzky, T., Schulte, M., Ludwig, A., Rose-John, S., Blobel, C., Hartmann, D., Altevogt, P., Saftig, P., and Reiss, K. 2005. L1 Is Sequentially Processed by Two Differently Activated Metalloproteases and Presenilin/{gamma}-Secretase and Regulates Neural Cell Adhesion, Cell Migration, and Neurite Outgrowth. Mol. Cell. Biol. 25: 9040-9053.
22. Landman, N., and Kim, T. W. 2004. Got RIP? Presenilin-dependent intramembrane proteolysis in growth factor receptor signaling. Cytokine Growth Factor Rev. 15: 337-351.
23. Cheng, L., Itoh, K., and Lemmon, V. 2005. L1-mediated branching is regulated by two ezrin-radixin-moezin (ERM)-binding sites, the RSLE region and a novel juxtamembrane ERM-binding region. J Neurosci. 25: 395-403.
24. Schaefer, A. W., Kamiguchi, H., Wong, E. V., Beach, C. M., Landreth, G., and Lemmon, V. 1999. Activation of the MAPK signal cascade by the neural cell adhesion molecule L1 requires L1 internalization. J. Biol. Chem. 274: 37965-37973.
25. Schaeffer, H. J., and Weber, M. J. 1999. Mitogen-activated protein kinases: specific messages from ubiquitous messengers. Mol. Cell. Biol. 19: 2435-2444.
26. Colucci-D'Amato, L, Perrone-Capano, C., and di Porzio, U. 2003. Chronic activation of ERK and neurodegenerative diseases. Bioessays. 25: 1085-1095.
27. Manor, D., Shmidt, E. N., Budhu, A., Flesken-Nikitin, A., Zgola, M., Page, R., Nikitin, A. Y., and Noy, N. 2003. Mammary carcinoma suppression by cellular retinoic acid binding protein-II. Cancer Res. 63: 4426-33.
28. Marambaud, P., Wen, P. H., Dutt, A., Shio,i J., Takashima, A., Siman, R., and Robakis, N. K. 2003. A CBP binding transcriptional repressor produced by the PS1/epsilon-cleavage of N-cadherin is inhibited by PSI FAD mutations. Cell 114: 635-645.
29. Schmidt-Zachmann, M. S., Knecht, S., and Kramer, A. 1998. Molecular characterization of a novel, widespread nuclear protein that colocalizes with spliceosome components. Mol. Biol. Cell 9: 143-160.
30. Gutwein, P., Stoeck, A., Riedle, S., Gast, D., Runz, S., Condon, T. P., Marme, A., Phong, M. C., Linderkamp, O., Skorokhod, A., and Altevogt, P. 2005. Cleavage of L1 in exosomes and apoptotic membrane vesicles released from ovarian carcinoma cells. Clin. Cancer Res. 11: 2492-2501.
31. Bar-Eli, M. 2001. Gene regulation in melanoma progression by the AP-2 transcription factor. Pigment Cell Res. 14: 78-85.
32. Lotan, R. 1996. Retinoids in cancer chemoprevention. FASEB J., 10: 1031-1039.
33. Morrison, D. K., and Davis, R. J. 2003. Regulation of MAP kinase signaling modules by scaffold proteins in mammals. Ann.u Rev. Cell Dev. Biol. 19: 91-118.
34. Wong, E. V., Schaefer, A. W., Landreth, G., and Lemmon, V. 1996. Casein kinase II phosphorylates the neural cell adhesion molecule L1. J. Neurochem. 66: 779-786.
35. Schmid, R. S., Pruitt, W. M., and Maness, P. F. 2000. A MAP kinase-signaling pathway mediates neurite outgrowth on L1 and requires Src-dependent endocytosis. J. Neurosci. 20: 4177-4188.
36. Ignelzi, M. A., Miller, D. R., Soriano, P., and Maness PF 1994. Impaired neurite outgrowth of src-minus cerebellar neurons on the cell adhesion molecule L1. Neuron 12:873-884.
37. Cheng, L., Lemmon, S., and Lemmon, V. 2005. RanBPM is an L1-interacting protein that regulates L1-mediated mitogen-activated protein kinase activation. J. Neurochem. 94: 1102-1110.
38. Kim, S. K., Park, H. J., Hong, H. S., Baik, E. J., Jung, M. W. and Mook-Jung, I. 2006. ERK1/2 is an endogenous negative regulator of the gamma-secretase activity. FASEB J. 20:157-159.
39. Tang, N., He, M., O'Riordan, M. A., Farkas, C., Buck, K., Lemmon, V. and Bearer, C. F. 2006. Ethanol inhibits L1 cell adhesion molecule activation of mitogen-activated protein kinases. J. Neurochem. 96: 1480-1490.
40. Moldenhauer, G., Momburg, F., Moller, P., Schwartz, R. and Hammerling, G. J. 1987. Epithelium-specific surface glycoprotein of Mr 34,000 is a widely distributed human carcinoma marker. Br. J. Cancer 56: 714-721.
41. Huszar, M., Moldenhauer, G., Gschwend, V., Ben-Arie, A., Altevogt, P. and Fogel, M. 2006. Expression profile analysis in multiple human tumors identifies L1 (CD171) as molecular marker for differential diagnosis and targeted therapy. Human Pathol. 37:1000-8.
42. Gutwein, P., Oleszewski, M., Mechtersheimer, S., Agmon-Levin, N., Krauss, K., and Altevogt, P. 2000. Role of Src kinases in the ADAM-mediated release of L1 adhesion molecule from human tumor cells. J. Biol. Chem. 275: 15490-15497.
43. Current protocols in Molecular Biology 1999. 21.3.1-21.3.12.
44. Brunner, G., Reimbold, K., Meissauer, A., Schirrmacher, V., and Erkell, L. J. 1998. Sulfated glycosaminoglycans enhance tumor cell invasion in vitro by stimulating plasminogen activation. Exp Cell Res. 239: 301-310.

### EXAMPLE 3

### Abbreviations

IL1β—Interleukin 1 beta; IL1-RA—Interleukin 1 receptor antagonist; iNOS—inducible nitric oxide synthase; NO—nitric oxide; PDAC—pancreatic ductal adenocarcinoma; PI—propidium iodide; RT—Reverse transcriptase; SNAP—S-Nitroso-N-acetyl-D,L-penicillamine

### Abstract

Pancreatic ductal adenocarcinoma (PDAC) is characterized by rapid tumor progression, high metastatic potential and profound chemoresistance. We recently reported that induction of a chemoresistant phenotype in the PDAC cell line PT45-P1 by long term chemotherapy involves an increased IL1β-dependent secretion of nitric oxide (NO) accounting for efficient caspase inhibition. In the present study we elucidated the involvement of L1CAM, an adhesion molecule previously found in other malignancies, in this NO-dependent chemoresistance. Chemoresistant PT45-P1res cells, but not chemosensitive parental PT45-P1 cells, express high levels of L1CAM in an ILβ-dependent fashion. PT45-P1res cells subjected to siRNA mediated L1CAM knock-down exhibited reduced iNOS expression and NO secretion as well as a significant increase of anti-cancer drug induced caspase activation, an effect reversed by the NO donor SNAP. Conversely, overexpression of L1CAM in PT45-P1 cells conferred anti-apoptotic protection to anti-cancer drug treatment. Interestingly, L1CAM ectodomain shedding, i.e. by ADAM10, as reported for other L1CAM related activities, seemed to be dispensable for anti-apoptotic protection by L1CAM. Neither the shedded L1CAM ectodomain was detected in chemoresistant L1CAM expressing PT45-P1 cells nor did the administration of various metalloproteinase inhibitors affect L1CAM-dependent chemoresistance. Immunohistochemical analysis revealed L1CAM expression in 80% of pancreatic cancer specimens supporting a potential role of L1CAM in the malignancy of this tumor. These findings substantiate our understanding of the molecular mechanisms leading to chemoresistance in PDAC cells and indicate the importance of L1 CAM in this scenario.

### Introduction

Pancreatic ductal adenocarcinoma (PDAC) is 4-5^{th} in the rank order of fatal tumor diseases in Western Countries with a 5 year survival rate <2% and a still increasing prevalence (Lockhart et al., 2005; Schneider et al., 2005). Due to its largely symptomeless progression, PDAC is diagnosed in an already advanced stage with widespread metastasis, and for 80-90% of the patients no option for a curative surgical resection exist anymore at the time of diagnosis. For these patients, current therapeutical options rely on chemotherapy treatment with 5-fluoruracil or gemcitabine, but solely with palliative intention. The failure of all chemotherapeutic strategies is largely based on the profound chemoresistance of PDAC cells that either results from preexisting intrinsic mechanisms or from an extrinsic induction by anti cancer drug treatment itself. Irrespective of these mechanisms, the capability of PDAC cells to evade the effect of cytostatic drugs mainly results from an efficient protection against drug induced apoptosis. We have previously shown (Muerkoster et al., 2004) that intense double paracrine interactions of PDAC cells with surrounding stromal fibroblasts led to the induction and manifestation of anti-apoptotic protection in these tumor cells involving an elevated IL1β dependent release of nitric oxide (NO). Both cellular mediators are also induced in PDAC cells after extended cytostatic drug exposure that similarly results in a chemoresistant phenotype (Sebens Muerkoster et al., 2006). Furthermore, the IL1β dependent NO secretion led to a broad inhibition of caspases i.e. caspase-3, -7, -8 and -9 in long-term drug treated PT45-P1res cells (Sebens Muerkoster et al., 2006). Since chemoresistant PT45-P1res cells also show altered adhesion properties, we elucidated whether the expression of certain adhesion molecules is functionally related to the gain of chemoresistance in PDAC. Meanwhile, a number of studies show that the chemosensitivity of cancer cells is affected by the extent of cell adhesion and expression of intercellular adhesion molecules (reviewed in St Croix & Kerbel, 1997). Miyamoto et al showed that acquired chemoresistance of pancreatic cancer cells depends on the expression of and adhesion to extracellular matrix proteins (Miyamoto et al., 2004). Recently, the adhesion molecule L1CAM/CD171 has attracted much attention since its expression is found in an increasing number of tumors, i.e. melanoma, glioma, ovarial and colon cancer, gastrointestinal stromal tumors or neuroendocrine pancreatic carcinoma (Gast et al., 2005; Gavert et al., 2005; Izumoto et al., 1996; Kaifi et al., 2006a; Kaifi et al., 2006b; Meier et al., 2006). In several tumors, high L1CAM expression could be associated with poor prognosis and short survival times (Fogel et al., 2003; Kaifi et al., 2006a; Kaifi et al., 2006b). L1 CAM was initially detected in neuronal cells where it is involved in several biological processes like neuron-neuron adhesion, neurite fasciculation, synaptogenesis, neurite outgrowth on Schwann cells and neuronal cell migration (Brumendorf et al., 1998; Hortsch, 2000; Schachner, 1997).

L1 CAM is a 200-220 kD glycoprotein and a member of the immunoglobulin superfamily. It consists of six immunoglobulin like domains at the amino terminal end of the molecule followed by five fibronectin type III homologous repeats, a single transmembrane region and a short intracellular domain (Moos et al., 1988). Beside its cell surface localization, L1CAM can also be cleaved by several proteases, i.e. the matrix metalloproteinases ADAM10 and ADAM17 or by γ-secretases (Maretzky et al., 2005). Soluble L1CAM has been reported to be important for migration of neuronal as well as of tumor cells (Maretzky et al., 2005; Mechtersheimer et al., 2001), and several studies support a role for L1CAM in tumor growth (Arlt et al., 2006), tumor cell invasion and metastasis of melanoma, ovarial and colon cancer (Fogel et al., 2003; Gavert et al., 2005; Mechtersheimer et al., 2001). Up to now, no data exist on L1CAM expression in PDAC and its role in the protection of drug induced apoptosis. Since Loers et al. showed that L1CAM mediated neuroprotection is associated with caspase inhibition (Loers et al., 2005), the aim of the present study was to investigate whether L1CAM is expressed in PDAC and whether it is involved in reduced caspase activation and, thereby, in chemoresistance of PDAC cells.

### Results

### L1CAM Expression in Chemoresistant PT45-P1res Cells is IL10 Dependent.

Chemoresistant PT45-P1res cells yielded from a six week treatment with low dose etoposide show altered adhesive properties in comparison with the parental chemosensitive cell line PT45-P1 (unpublished observations). We therefore analysed the involvement of adhesion molecules in the chemoresistance of these cells. Interestingly, PT45-P1res cells exhibit a much higher expression of the adhesion molecule L1CAM than PT45-P1 cells, as shown by western blotting (FIG. 15a) and real-time PCR (FIG. 16a). Furthermore, immunostaining and subsequent flow cytometry revealed a distinct L1CAM surface expression in PT45-P1res and PT45-P1 cells (FIG. 15b). Since we have recently shown, that IL1β is an important mediator of chemoresistance in PT45-P1res cells (Sebens Muerkoster et al., 2006), we next analysed whether L1CAM expression is IL1, dependent. Inhibition of IL1 signalling by treatment with the IL1-receptor-antagonist (IL1-RA) reduced L1CAM expression in PT45-P1res cells on mRNA (FIG. 16a) as well as on protein level (FIG. 16b) indicating IL1β dependent upregulation of L1CAM expression after long-term drug exposure. In support of these data, treatment of chemosensitive PT45-P1 cells with 20 ng/mL IL1β enhanced L1CAM expression (FIG. 16a+b).

### L1 CAM is Involved in the Mediation of Chemoresistance in PT45-P1res Cells.

To verify that L1CAM is directly involved in the mediation of chemoresistance, its expression in PT45-P1res cells was blocked by siRNA treatment. Two different L1CAM specific siRNAs were positively tested for reducing L1CAM expression along with an increase of etoposide induced caspase-3/-7 activity (FIG. 17a). Owing to its greater sensitizing potential, the L1CAM specific siRNA-2 was used for further experiments. As shown by L1CAM immunostaining and flow cytometry (FIG. 17b) treatment with this siRNA also reduced L1CAM surface expression. The specificity of L1CAM siRNA was verified by the detection of av integrin expression in PT45-P1res cells exhibiting unaltered levels after transfection with control or L1CAM siRNA (FIG. 17c).

Moreover, L1CAM knock down led to a significant apoptosis induction in these cells after treatment with anti-cancer drugs as determined by annexinV staining (FIG. 17d, left panel) or by a luminescent caspase-3/-7 activity assay (FIG. 17d, right panel). In comparison with control siRNA transfectants, etoposide and gemcitabine induced caspase-3/-7 activity was increased by 48% and 50%, respectively, and annexinV staining was raised by 63% and 67%, respectively. Furthermore, treatment with an anti L1CAM antibody prior to exposure to the cytostatic drug similarly abolished chemoresistance of PT45-P1res cells as analysed by annexinV staining and caspase-3/-7 activity, respectively (FIG. 17e).

Similar findings were obtained with two other chemoresistant PDAC cell lines. In Colo357 and Panc1 cells that exhibit high L1CAM expression, the siRNA mediated knock down of L1CAM (FIG. 18a) led to the sensitization towards treatment with etoposide (FIG. 18b) as shown by an increased (64% and 85%, respectively) casapse-3/-7 activity.

In accordance with these data, overexpression of L1CAM (FIG. 19a, b) significantly reduced the sensitivity of PT45-P1 cells towards cytostatic drug induced apoptosis in comparison with mock transfected cells (FIG. 19c). In response to treatment with etoposide and gemcitabine, annexinV staining was decreased by 34% and 30%, respectively, and caspase-3/-7 activity was reduced by 32% and 38%, respectively. These findings support a role of L1 CAM in the gain and manifestation of chemoresistance.

### L1CAM Cleavage is Dispensable for Induction of Chemoresistance in PT45-P1res Cells.

Since several biological functions of L1CAM depend on its ectodomain cleavage by certain proteinases, yielding soluble L1CAM, we investigated whether L1CAM cleavage is essential for chemoresistance induction. For this purpose, PT45-P1res cells were either left untreated or treated with the matrix metalloproteinase inhibitors Tapi-0, Tapi-1 or GM6001 or with the γ-secretase inhibitor L685,458. After 24 hours, cellular lysates were analysed for L1 CAM cleavage by using either the monoclonal antibody UJ127 from Acris, detecting the extracellular part of the protein or the pcytL1 antibody recognizing the cytoplasmic part of the full length form of L1CAM and of the C-terminal fragment emerging from proteinase cleavage. Incubation of PT45-P1res cells with neither of the inhibitors changed L1 CAM expression as indicated by the constant amounts of the full-length form (220 kDa) of L1CAM as well as of its cytoplasmic 32 kD fragment (FIG. 20a). Furthermore, these inhibitors did not significantly affect apoptosis induction after etoposide treatment as determined by caspase-3/-7 assay (FIG. 20b). In line with these findings, incubation of L1CAM transfected PT45-P1 cells with the matrix metalloproteinase inhibitors or L685,458 did neither influence the expression of full-length L1CAM nor increased the level of the 32 kD fragment (FIG. 20c) nor affect the apoptosis inducing effect of etoposide (FIG. 20d). These data indicate that L1CAM cleavage is dispensable for mediation of chemoresistance.

L1 CAM Mediates iNOS Induction and NO Release in PT45-P1res Cells.

In order to proof whether L1CAM mediated chemoresistance is linked to enhanced NO release and subsequent caspase inhibition in PT45-P1res cells, as we have recently demonstrated (Sebens Muerkoster et al., 2006), iNOS mRNA expression and NO release were analyzed in PT45-P1res cells subjected to L1CAM knock down. As shown in FIG. 21a, transfection of PT45-P1res cells with L1CAM siRNA clearly reduced the amount of iNOS mRNA. Accordingly, NO levels were significantly diminished in cell culture supernatants of PT45-P1res cells after L1CAM knock down compared to control transfected PT45-P1res cells (from 4.9 to 0.9 μmol/10⁵ cells; FIG. 21b). Furthermore, NO levels could be decreased in control siRNA transfected PT45-P1res cells by IL1-RA treatment, whereas in these cells with already diminished NO formation during L1CAM knock down no further reducing effect of the IL1-RA on NO levels was observed (FIG. 21b). Accordingly, etoposide induced caspase activation was increased in control transfected PT45-P1res cells by ILL-RA treatment but not in L1CAM siRNA transfected cells (FIG. 21c). These data underscore the involvement of L1CAM in the IL1β dependent NO secretion and chemoresistance induction in PT45-P1res cells.

### Chemoresistance of PT45-P1res Cells Depends on L1 CAM Mediated NO Secretion.

Next, it was further analysed whether the induction of chemoresistance by L1CAM depends on L1CAM mediated NO secretion. For this purpose, L1CAM expression was suppressed by siRNA transfection in PT45-P1res cells subjected to treatment with etoposide in the absence or presence of the NO donor S-Nitroso-N-acetyl-D,L-penicillamine (SNAP). As shown by caspase-3/-7 assay, SNAP treatment restored the chemoresistant phenotype in PT45-P1res cells after L1CAM knock down (FIG. 22). Whereas L1CAM siRNA transfected cells showed a 2.3-fold induction in caspase-3/-7 activity after etoposide treatment compared to 1.6-fold induction in control-siRNA transfectants, additional SNAP treatment completely reversed the increased caspase activity during L1CAM knock-down, thus restoring the chemoresistant phenotype. These data indicate that L1 CAM mediated NO release contributes to the induction of chemoresistance in PT45-P1 cells.

### L1 CAM is Expressed Ductal Pancreatic Adenocarcinoma.

Finally, tissue sections of human pancreatic adenocarcinomas from 20 patients were analysed for L1CAM expression. In 16 tumor samples (=80%) L1CAM expression was detectable, showing moderate or strong expression in 5 sections (Table 1, FIG. 23). In peritumoral areas, nerves and germinal centers of lymph nodes were intensely stained, whereas normal epithelial cells exhibited no L1CAM expression, at all. Interestingly, the strongest L1CAM expression could be detected in grade 3 tumors (Table 1). These data imply that L1CAM is an important mediator in the malignant transformation and thereby in chemoresistance of pancreatic adenocarcinoma.

### Discussion

The present study shows, for the first time, that the adhesion molecule L1CAM plays an important role in the induction and manifestation of chemoresistance in PDAC cells. Until now, chemoresistance is the major reason for the desperately poor therapeutical outcome in the treatment of this tumor. Hence, a better understanding of the precise mechanisms leading to drug resistance is a prerequisite for substantial improvement of current therapeutical strategies. Recently, we identified a complex mechanism by which PDAC cells gain efficient protection from drug induced apoptosis involving the increased secretion of IL1 (Arlt et al., 2002; Muerkoster et al., 2004; Sebens Müerkoster et al., 2006). Thus in long-term drug treated PDAC cells elevated IL1β levels induce iNOS expression and subsequent release of NO resulting in cysteine nitrosylation of caspases and, thereby, in their inhibition (Sebens Muerkoster et al., 2006). As shown by the present data, the adhesion molecule L1CAM is involved in IL1β mediated NO release and the resulting caspase inhibition, thus providing an interesting link between cell adhesion and apoptosis protection. Whereas chemosensitive PT45-P1 cells express only little L1CAM, long-term drug treatment increased expression levels of L1CAM in chemoresistant PT45-P1res cells. Interestingly, increased L1CAM expression has been similarly seen in chemoresistant Colo357 and Panel cells as well as in PT45-P1 and T3M4 cells derived from continuous coculture with pancreatic stromal fibroblasts, thereby gaining a chemoresistant phenotype (unpublished observations). Drug-induced L1CAM expression seems to be dependent on IL1β since treatment with the IL1-RA diminished L1CAM levels in PT45-P1res cells and knock down experiments with specific L1CAM siRNA underlined the importance of L1CAM in the induction of chemoresistance in these cells. This close relation between the expression of L1CAM and IL1β is also indicated by the fact that particularly grade-2 and grade-3 tumors exhibit most intensive immunostaining not only for L1CAM (table 1) but also for IL1β, as shown recently (Müerköster et al., 2004). Moreover, L1CAM transfection of PT45-P1 cells significantly decreased chemosensitivity towards cytostatic drug treatment supporting the role for L1CAM in protection from drug-induced apoptosis. Loers et al. could demonstrate that neuritogenesis and neuroprotection from oxidative stress and staurosporine treatment are both dependent on L1CAM expression (Loers et al., 2005).

Interestingly, proteolytic cleavage of L1CAM which is prerequisite for neuroprotection and also for mediation of cell migration and invasion (Gast et al., 2005; Mechtersheimer et al., 2001) is obviously not essential for mediation of chemoresistance. Neither the broad spectrum matrix metalloproteinase inhibitors Tapi-0, Tapi-1 and GM6001, respectively, nor the γ-secretase inhibitor L685,458 affected the expression of the full-length membrane bound form of L1CAM. Additionally, the faint expression of the cytoplasmic 32 kD L1CAM fragment which is originated after cleavage did not differ between PT45-P1res and PT45-P1 cells and did not change upon treatment with these inhibitors. Furthermore, none of the soluble fragments could be detected in supernatants of these differently treated PT45-P1res cells (data not shown).

L1 CAM triggered neuroprotection has been shown to be associated with increased phosphorylation of ERK1/2, Akt und Bad as well as inhibition of caspase-9 (Loers et al., 2005). In contrast, PT45-P1res cells that exhibit increased L1CAM expression and an impaired activity of the initiator caspases-8 and -9 as well as the effector caspases -3 and -7, accounting for anti-apoptotic protection against cytostatic drugs, do not show significant changes in Akt and ERK1/2 phosphorylation (data not shown).

As we recently demonstrated, this broad caspase inhibition in PT45-P1res cells is apparently caused by nitrosylation of cysteine residues in the active site of these enzymes (Sebens Muerkoster et al., 2006). Nitrosylation is mediated by IL1β induced NO levels in chemoresistant PDAC cells, a process obviously dependent on L1CAM. Inhibition of L1CAM expression significantly reduced iNOS induction and NO release in PT45-P1res cells thereby enhancing caspase activation and apoptosis induction. Furthermore, L1CAM knock down clearly impaired induction of iNOS mRNA level and NO secretion by IL1β, Castellani et al. demonstrated that interaction of L1CAM with Neuropilin-1, a receptor for semaphorins and a coreceptor for VEGF, leads to enhanced NO synthesis (Castellani et al., 2002). NO synthesis can be induced by interaction of L1CAM and Neuropilin-1 on one cell (cis) or by interaction of both molecules on different cells (trans). Moreover, it has been shown that PDAC cells highly express neuropilin-1 (Fukahi et al., 2004) and that overexpression of neuropilin-1 is able to induce chemoresistance in PDAC cells (Wey et al., 2005). Since PT45-P1res and PT45-P1 cells both exhibit Neuropilin-1 expression (unpublished observation), it seems likely that cis and trans interactions of Neuropilin-1 with L1CAM increase iNOS expression and NO synthesis, thereby leading to chemoresistance in PT45-P1res cells. The fact that immunohistochemical analysis of PDAC sections revealed strong L1 CAM expression in 80% of the tumor samples and that chemoresistant PDAC cells derived from coculture with stromal fibroblasts also exhibit increased L1CAM expression underscore the role of L1CAM in the induction of chemoresistance in this tumor entity.

Besides its role in the gain of chemoresistance, L1CAM might also be of importance for invasion and metastasis of PDAC cells, a role which has to be defined yet. Taking all these findings into account, L1CAM represents an interesting therapeutic target to overcome chemoresistance and to concomitantly interfere with the process of metastasis.

### Material and Methods

### Cell Lines and Culture.

The human PDAC cell line PT45-P1 as well as its handling were described previously (Kalthoff et al., 1993). PT45-P1 and PT45-P1res cells were kept in culture (37° C., 5% CO₂, 85% humidity) using RPMI 1640 medium (PAA Laboratories, Colbe, Germany) supplemented with 1% glutamine (Life Technologies, Eggenstein, Germany) and 10% FCS (Biochrom KG, Berlin, Germany). The generation of PT45-P1res cells was done as described elsewhere (Sebens Muerkoster et al., 2006). The human PDAC cell lines Colo357 and Panc1 were kindly provided by H. Kalthoff (UKSH-Campus Kiel) and kept in culture using RPMI 1640 medium supplemented with 1% glutamine, 10% FCS and 1% sodium pyrovate (Biochrom).

Antibodies and Reagents.

Recombinant human IL-1β and the IL-1 receptor antagonist (IL1-RA) were obtained from R&D Systems (Wiesbaden, Germany). The matrix metalloproteinase inhibitors GM6001, Tapi-0 and Tapi-1 were obtained from Calbiochem (via Merck Biosciences, Schwalbach/Ts, Germany) and the γ-secretase inhibitor L685,458 was purchased from Sigma-Aldrich Chemie (Taufkirchen, Germany). S-Nitroso-N-acetyl-D,L-penicillamine (SNAP) was purchased from Alexis (Grunberg, Germany). Etoposide was purchased from Bristol Myers Squibb (München, Germany) and gemcitabine from Lilly (Bad Homburg, Germany). The following antibodies were used for the detection of L1CAM by western blotting: Mouse monoclonal anti L1CAM detecting the full-length 220 kD molecule, soluble 85 kD and 200 kD fragments (clone UJ127 from Acris Antibodies, Hiddenhausen, Germany) and a rabbit polyclonal anti pcytL1 antibody detecting the cytoplasmic part of L1CAM (220 kD, 85 kD, 32 kD fragments) as described previously (Mechtersheimer et al., 2001). For blocking experiments and flow cytometry analysis, the monoclonal mouse L1-11A antibody anti L1CAM (subclone of UJ127.11) was used (Mechtersheimer et al., 2001). Maus IgG control antibody was obtained from Chemicon (Hampshire, United Kingdom).

### AnnexinV/PI Staining and Caspase-3/-7 Assay.

As described elsewhere (Sebens Müerköster et al., 2006), apoptosis was determined by staining with annexinV/propidium iodide (Biocarta, Hamburg, Germany) and subsequent fluorescence flow cytometry (GalaxyArgon Plus; DAKO Cytomation, Hamburg, Germany) using the FLOMAX software, and by the detection of caspase-3/-7 activity using a homogeneous luminescent assay (Promega, Mannheim, Germany). All samples were measured in duplicates.

### Cell Transfection.

For L1CAM transfection, PT45-P1 cells were seeded into 6 well plates (2×10⁵ cells/well), were grown overnight, followed by transfection with 5 μL/well DIMRIE reagent (Invitrogen) and 0.6 μg/well of the following plasmids: pcDNA3.1 (mock) or pcDNA3.1-L1CAM (L1CAM). Upon transfection for 18 hours, 1 mL medium containing 20% FCS was added and cells were left untreated or were treated as indicated for further 24 hours. For knock down of L1CAM, PT45-P1res cells were seeded into 12 well plates (1×10⁵ cells/well), were grown overnight followed by transfection with 12 μL/well RNAiFect reagent (Invitrogen) and 2 μg/well of either Stealth negative control siRNA (Invitrogen) or Stealth L1CAM siRNA (Invitrogen). After overnight transfection, cells were either left untreated or treated as indicated for further 24 hours.

### L1 CAM Surface Staining and Flow Cytometry.

After washing in PBS, cells grown in 6-well culture plates were detached with 5 mmol/L EDTA in PBS and then washed with PBS once again. Blocking was conducted in 1% BSA/PBS for 60 min. at room temperature followed by incubation with the anti L1CAM antibody L1-11A or an isotype matched control antibody (1:1000, in 1% BSA/PBS) at 4° C. overnight. Then, cells were washed three times in PBS followed by incubation (60 min, 37° C.) with a goat-anti mouse antibody conjugated with Alexa flour 488 (Dianova, Hamburg, Germany) diluted 1:500 in 1% BSA/PBS. After washing in PBS, cells were resuspended in 500 μL PBS and analysed by fluorescence flow cytometry.

### NO-Assay.

Cells were seeded for transfection and cultured as described above. 48 hours after transfection, supernations were taken and precleared by centrifugation (5000 rpm, 10 min.) prior to analysis. NO secreted into cell culture supernatants was quantified using the Total nitric oxide (NO) calorimetric assay (R&D Systems). The assay was performed following the manufacturer's instructions. Concentrations of measured NO were normalized to the cell numbers determined in parallel.

### Western Blotting.

Cells were seeded into 6 well and 12 well plates, respectively, and transfected or treated as indicated. Then, cells were washed once with PBS and lysed with 1 volume of 2×SDS sample buffer (128 mmol/L Tris-Base, 4.6% SDS, 10% glycerol). Samples were heated for 5 minutes at 95° C. and put on ice for 2 minutes. Protein concentrations were determined using the D_{c} Protein assay (BioRad). Ten μg of protein adjusted to an appropriate volume of SDS sample buffer containing 0.2 mg/mL bromphenolblue (Serva, Heidelberg, Germany) and 2.5% β-mercaptoethanol (Biomol, Hamburg, Germany) were submitted to electrophoresis on a 4-20% ProGel-Tris-glycin-gel (Anamed, Darmstadt, Germany) and immunoblotting was performed as described previously (Arlt et al, 2001). For detection of L1CAM, a monoclonal antibody (clone UJ127 from Acris Antibodies) was diluted at a concentration of 0.4 μg/mL in 5% nonfat milk powder and 0.05% Tween in TBS (blotto) and incubated overnight at 4° C. For detection of the cytoplasmic domain of L1CAM, the pcytL1 antibody (Mechtersheimer et al., 2001) was used at a concentration of 1 μg/mL in blotto and incubated overnight at 4° C. As control of equal protein load, a polyclonal rabbit antibody for HSP90 (Santa Cruz, Heidelberg, Germany) was diluted 1:2000 in blotto. The mouse anti CD51 antibody from Beckman Coulter GmbH (Krefeld, Germany) was used at a concentration of 1:500 in blotto for detection of human αv integrin. Incubation with the primary antibodies was performed overnight at 4° C. For detection of the primary antibodies, anti-mouse and anti-rabbit HRP-linked antibodies (Cell Signaling), respectively, were used at a dilution of 1:2000 in blotto-TBST at room temperature for 1 hour. After washing in TBST, blots were developed using the LumiGlo peroxidase detection kit (Cell Signaling).

### Real-Time PCR.

2 μg of total RNA were reverse-transcribed into single-stranded cDNA, as described previously (Schafer et al., 1999). Two μL of cDNA and 0.2 μmol/L gene-specific primers were adjusted with RNAse-free water to a volume of 15 μL. To this mixture, 15 μL of iQ SYBR Green Supermix (BioRad) were added. Primers for the detection of L1CAM (Gavert et al., 2005) were used under the following conditions: 95° C./1 min; 95° C./1 min, 52° C./30 see, 72° C./30 see for 40 cycles; 72° C./10 min. Primers for the detection of iNOS were from Biosource (Ratingen, Germany) and used under the following PCR conditions: 95° C./5 min; 95° C./45 sec, 60° C./45 sec, 72° C./45 sec for 40 cycles; 72° C./10 min. For control, β-actin was amplified in parallel using primers from BD Biosciences Clontech. The Real-time PCR was performed with a MyiQ Single Color Real-time PCR Detection System (BioRad). Data were collected during annealing steps and were further analysed by using the i-Cycler iQ Optical system software (BioRad). All samples were analysed in duplicates and data are expressed as amount of mRNA in arbitrary units.

### Immunohistochemistry.

20 ductal PDAC tissues obtained from surgical specimens according to a protocol approved by the ethics committee of the University Hospitals, Kiel (Permission number 110/99) were investigated. Routinely processed formalin-fixed sections of human ductal adenocarcinomas were blocked with 0.03% H₂O₂, followed by heat-mediated antigen retrieval using TRIS-EDTA-citrate buffer in a pressure cooker for 3 min. Ten μg/mL mouse monoclonal anti-L1CAM antibody (Acris Antibodies) were applied as primary antibody. The reaction was detected by avidin-biotin-peroxidase using the Vectastain-ABC Kit (Vector Laboratories, Burlingame, Calif., USA). For negative control, the primary antibody was omitted. The immunohistochemical reactions were semiquantitatively scored as mild (<10% of the tumor cells stained), moderate (10-50%), and strong (>50%).

### Statistics.

Data are presented as mean±SD and analyzed by Student's t-test. A p-value <0.05 (indicated as * in the figures) was considered as statistically significant.

### LITERATURE

Arlt A, Vomdamm J, Muerkoster S, Yu H, Schmidt W E, Folsch U R, et al. (2002). Autocrine production of interleukin 1beta confers constitutive nuclear factor kappaB activity and chemoresistance in pancreatic carcinoma cell lines. Cancer Res, 62, 910-6.
Arlt M J, Novak-Hofer I, Gast D, Gschwend V, Moldenhauer G, Grunberg J, et al. (2006). Efficient inhibition of intra-peritoneal tumor growth and dissemination of human ovarian carcinoma cells in nude mice by anti-L1-cell adhesion molecule monoclonal antibody treatment. Cancer Res, 66, 936-43.
Brummendorf T, Kenwrick S and Rathjen F G. (1998). Neural cell recognition molecule L1: from cell biology to human hereditary brain malformations. Curr Opin Neurobiol, 8, 87-97.
Castellani V, De Angelis E, Kenwrick S and Rougon G. (2002). Cis and trans interactions of L1 with neuropilin-1 control axonal responses to semaphorin 3A. Embo J, 21, 6348-57.
Fogel M, Gutwein P, Mechtersheimer S, Riedle S, Stoeck A, Smirnov A, et al. (2003). L1 expression as a predictor of progression and survival in patients with uterine and ovarian carcinomas. Lancet, 362, 869-75.
Fukahi K, Fukasawa M, Neufeld G, Itakura J and Korc M. (2004). Aberrant expression of neuropilin-1 and -2 in human pancreatic cancer cells. Clin Cancer Res, 10, 581-90.
Gast D, Riedle S, Schabath H, Schlich S, Schneider A, Issa Y, et al. (2005). L1 augments cell migration and tumor growth but not beta3 integrin expression in ovarian carcinomas. Int J Cancer, 115, 658-65.
Gavert N, Conacci-Sorrell M, Gast D, Schneider A, Altevogt P, Brabletz T, et al. (2005). L1, a novel target of beta-catenin signaling, transforms cells and is expressed at the invasive front of colon cancers. J Cell Biol, 168, 633-42.
Hortsch M. (2000). Structural and functional evolution of the L1 family: are four adhesion molecules better than one? Mol Cell Neurosci, 15, 1-10.
Izumoto S, Ohnishi T, Arita N, Hiraga S, Taki T and Hayakawa T. (1996). Gene expression of neural cell adhesion molecule L1 in malignant gliomas and biological significance of L1 in glioma invasion. Cancer Res, 56, 1440-4.
Kaifi J T, Strelow A, Schurr P G, Reichelt U, Yekebas E F, Wachowiak R, et al. (2006a). L1 (CD171) is highly expressed in gastrointestinal stromal tumors. Mod Pathol, 19, 399-406.
Kaifi J T, Zinnkann U, Yekebas E F, Schurr P G, Reichelt U, Wachowiak R, et al. (2006b). L1 is a potential marker for poorly-differentiated pancreatic neuroendocrine carcinoma. World J Gastroenterol, 12, 94-8.
Kalthoff H, Schmiegel W, Roeder C, Kasche D, Schmidt A, Lauer G, et al. (1993). p53 and K-RAS alterations in pancreatic epithelial cell lesions. Oncogene, 8, 289-98.
Lockhart A C, Rothenberg M L and Berlin J D. (2005). Treatment for pancreatic cancer: current therapy and continued progress. Gastroenterology, 128, 1642-54.
Loers G, Chen S, Grumet M and Schachner M. (2005). Signal transduction pathways implicated in neural recognition molecule L1 triggered neuroprotection and neuritogenesis. J Neurochem, 92, 1463-76.
Maretzky T, Schulte M, Ludwig A, Rose-John S, Blobel C, Hartmann D, et al. (2005). L1 is sequentially processed by two differently activated metalloproteases and presenilin/gamma-secretase and regulates neural cell adhesion, cell migration, and neurite outgrowth. Mol Cell Biol, 25, 9040-53.
Mechtersheimer S, Gutwein P, Agmon-Levin N, Stoeck A, Oleszewski M, Riedle S, et al. (2001). Ectodomain shedding of L1 adhesion molecule promotes cell migration by autocrine binding to integrins. J Cell Biol, 155, 661-73.
Meier F, Busch S, Gast D, Goppert A, Altevogt P, Maczey E, et al. (2006). The adhesion molecule L1 (CD171) promotes melanoma progression. Int J Cancer, 119, 549-55.
Miyamoto H, Murakami T, Tsuchida K, Sugino H, Miyake H and Tashiro S. (2004). Tumor-stroma interaction of human pancreatic cancer: acquired resistance to anticancer drugs and proliferation regulation is dependent on extracellular matrix proteins. Pancreas, 28, 38-44.
Moos M, Tacke R, Scherer H, Teplow D, Fruh K and Schachner M. (1988). Neural adhesion molecule L1 as a member of the immunoglobulin superfamily with binding domains similar to fibronectin. Nature, 334, 701-3.
Muerkoster S, Wegehenkel K, Arlt A, Witt M, Sipos B, Kruse M L, et al. (2004). Tumor stroma interactions induce chemoresistance in pancreatic ductal carcinoma cells involving increased secretion and paracrine effects of nitric oxide and interleukin-1beta. Cancer Res, 64, 1331-7.
Schachner M. (1997). Neural recognition molecules and synaptic plasticity. Curr Opin Cell Biol, 9, 627-34.
Schafer H, Arlt A, Trauzold A, Hunermann-Jansen A and Schmidt WE. (1999). The putative apoptosis inhibitor IEX-1L is a mutant nonspliced variant of p22(PRG1/IEX-1) and is not expressed in vivo. Biochem Biophys Res Commun, 262, 139-45.
Schneider G, Siveke J T, Eckel F and Schmid R M. (2005). Pancreatic cancer: basic and clinical aspects. Gastroenterology, 128, 1606-25.
Sebens Muerkoster S, Lust J, Arlt A, Hasler R, Witt M, Sebens T, et al. (2006). Acquired chemoresistance in pancreatic carcinoma cells: induced secretion of IL-1beta and NO lead to inactivation of caspases. Oncogene, 25, 4628.
St Croix B and Kerbel R S. (1997). Cell adhesion and drug resistance in cancer. Curr Opin Oncol, 9, 549-56.
Wey J S, Gray M J, Fan F, Belcheva A, McCarty M F, Stoeltzing O, et al. (2005). Overexpression of neuropilin-1 promotes constitutive MAPK signalling and chemoresistance in pancreatic cancer cells. Br J Cancer, 93, 233-41.

TABLE 1
L1CAM expression in ductal pancreatic adenocarcinoma.

| Record | | | | T- | N- | |
|---|---|---|---|---|---|---|
| No. | L1CAM | Age | Gender | stage* | stage* | Grade |
| 1 | + | 63 | m | 3 | 0 | 2 |
| 2 | − | 49 | m | 3 | 1 | 2 |
| 3 | ++ | 62 | f | 3 | 1 | 2 |
| 4 | + | 69 | f | 3 | 0 | 2 |
| 5 | + | 65 | m | 3 | 1 | 2 |
| 6 | + | 68 | m | 3 | 1 | 3 |
| 7 | +++ | 60 | m | 3 | 1 | 3 |
| 8 | + | 51 | m | 3 | 1 | 3 |
| 9 | ++ | 62 | f | 3 | 1 | 2 |
| 10 | + | 50 | m | 3 | 1 | 2 |
| 11 | + | 63 | f | 3 | 1 | 2 |
| 12 | + | 76 | f | 3 | 1 | 2 |
| 13 | − | 64 | f | 3 | 1 | 2 |
| 14 | − | 38 | f | 3 | 1 | 1 |
| 15 | ++ | 59 | f | 3 | 0 | 3 |
| 16 | + | 76 | f | 3 | 0 | 3 |
| 17 | + | 36 | f | 3 | 0 | 2 |
| 18 | − | 71 | f | 3 | 1 | 1 |
| 19 | + | 80 | m | 3 | 0 | 3 |
| 20 | +++ | 68 | m | 3 | 0 | 3 |

The immuno-histochemical L1CAM staining intensity:
+ = <10%,
++ = 10-50%,
+++ = >50% of the tumor cells stained.
− = no L1CAM staining in tumor cells.
*according to L.H. Sobin, Ch. Wittekind (eds.): TNM Classification of Malignant Tumors Sixth Edition 2002, Wiley-Liss Incorp.

### EXAMPLE 4

### Material and Methods

### Cell Lines and Culture

The human colon adenocarcinoma cell line CaCo2 were purchased from the German Collection of Microorganisms and Cell Cultures (Braunschweig, Germany) and the human glioblastoma cell line α98g was kindly provided by Peter Altevogt (Heidelberg, Germany). Both cell lines were kept under the following cell culture conditions: 37° C., 5% CO₂, 85% humidity. For the culture of CaCo2 cells, MEM medium (PAA Laboratories, Colbe, Germany) supplemented with 1% glutamine (Gibco Life Technologies, Eggenstein, Germany), 20% FCS (Biochrom KG, Berlin, Germany) and 1% nonessential amino acids (Gibco Life Technologies) and α98 g cells were cultured in DMEM medium (PAA Laboratories) supplemented with 1% glutamine (Gibco Life Technologies) and 10% FCS (Biochrom KG).

### AnnexinV/PI Staining and Caspase-3/-7 Assay

As described elsewhere (Sebens Müerköster et al., 2006), apoptosis was determined by staining with annexinV/propidium iodide (Biocarta, Hamburg, Germany) and subsequent fluorescence flow cytometry (GalaxyArgon Plus; DAKO Cytomation, Hamburg, Germany) using the FLOMAX software, and by the detection of caspase-3/-7 activity using a homogeneous luminescent assay (Promega, mannheim, Germany). All samples were measured in duplicates.

### Results

Pre-treatment of α98 g cells with the anti-L1 antibody L1-11A led to a sensitization towards etoposide and gemcitabine induced apoptosis as determined by caspase-3/-7 assay (FIG. 24) and AnnexinV binding (FIG. 25). In CaCo2 cells, L1-11A increased etoposide induced caspase-3/-7 activity compared to CaCo2 cells treated with a control antibody (FIG. 24).

1. Use of an L1 interfering molecule for the preparation of a medicament for sensitizing tumor cells in a patient for the treatment with a chemotherapeutic drug or with radiotherapy.

2. The method of claim 1, wherein the cells are at least partially resistant to the treatment with said chemotherapeutic drug or to radiotherapy.

3. The method of claim 1, wherein after the sensitization with the L1 interfering molecule the patient is further treated with said chemotherapeutic drug or with radiotherapy.

4. The method of claim 1, wherein during the sensitization with the L1 interfering molecule the patient is further treated with said chemotherapeutic drug or with radiotherapy.

5. The method of claim 1, wherein said L1 interfering molecule is selected from the group consisting of anti-L1 antibodies, antibody fragments thereof, siRNA, antisense RNA or DNA, ribozymes, low molecular weight molecules, and anticalins.

6. The method of claim 5, wherein said L1 interfering molecule is an anti-L1 antibody or an antibody fragment thereof.

7. The method of claim 6, wherein the antibody binds both soluble and membrane bound L1.

8. The use of method of claim 1, wherein the tumor cells are of a type selected from the group consisting of astrocytoma, oligodendroglioma, meningioma, neurofibroma, glioblastoma, ependymoma, Schwannoma, neurofibrosarcoma, medulloblastoma, melanoma cells (e.g. malignant melanoma), pancreatic cancer cells, prostate carcinoma cells, head and neck cancer cells, breast cancer cells, lung cancer cells (e.g. small cancer, non-small cancer), colon cancer cells (e.g. adenocarcinoma of the colon), colorectal cancer cells, gastrointestinal stromal tumor cells, ovarian cancer cells, endometrial cancer cells, renal cancer cells, neuroblastomas, squamous cell carcinomas, medulloblastomas, hepatoma cells and mesothelioma, epidermoid carcinoma, clear cell adenocarcinoma cells and serous adenocarcinoma of the uterine corps cells, cervix carcinoma cells, urinary tract adenocarcinoma cells, Pheochromocytoma cells, neuroma cells, neurillemoma cells, and paranganglioma cells.

9. The method of claim 1, wherein the tumor cells are epithelial tumor cells, preferably ovarian cancer cells, endometrial cancer cells, adenocarcinoma of the colon, pancreatic carcinoma cells or small cell lung cancer cells, or wherein the tumor cells are melanoma cells.

10. Use of an L1 interfering molecule for the preparation of a medicament for the treatment of tumor cells in a patient previously treated with a chemotherapeutic drug or with radiotherapy.

11. The method of claim 10, wherein the patient is at least partially resistant to the treatment with said chemotherapeutic drug or with radiotherapy.

12. Use of an L1 interfering molecule for the preparation of a medicament for the treatment of tumor cells in a patient at least partially resistant to treatment with a given chemotherapeutic drug or with radiotherapy.

13. Use of a L1 interfering molecule for the preparation of a medicament for the treatment of tumor cells in a patient, wherein the L1 interfering molecule is administered in combination with a chemotherapeutic drug or with radiotherapy.

14. The method of claim 13, wherein the chemotherapeutic drug or the radiotherapy is administered prior to the L1 interfering molecule.

15. The method of claim 13, wherein said L1 interfering molecule is selected from the group consisting of anti-L I antibodies, antibody fragments thereof, siRNA, antisense RNA or DNA, ribozymes, low molecular weight molecules, and anticalins.

16. The method of claim 15, wherein said L1 interfering molecule is anti-L1 antibody or an antibody fragment thereof.

17. The method of claim 16, wherein the antibody binds both soluble and membrane bound L1.

18. The method of claim 13, wherein the L1 interfering molecule is further linked to a toxin.

19. The method of claim 13, wherein the tumor cells are of a type selected from the group consisting of astrocytoma, oligodendroglioma, meningioma, neurofibroma, glioblastoma, ependymoma, Schwannoma, neurofibrosarcoma, medulloblastoma, melanoma cells (e.g. malignant melanoma), pancreatic cancer cells, prostate carcinoma cells, head and neck cancer cells, breast cancer cells, lung cancer cells (e.g. small cancer, non-small cancer), colon cancer cells (e.g. adenocarcinoma of the colon), colorectal cancer cells, gastrointestinal stromal tumor cells, ovarian cancer cells, endometrial cancer cells, renal cancer cells, neuroblastomas, squamous cell carcinomas, medulloblastomas, hepatoma cells and mesothelioma, epidermoid carcinoma, clear cell adenocarcinoma cells and serous adenocarcinoma of the uterine corps cells, cervix carcinoma cells, urinary tract adenocarcinoma cells, Pheochromocytoma cells, neuroma cells, neurillemoma cells, and paranganglioma cells.

20. The method of claim 13, wherein the tumor cells are epithelial tumor cells, preferably ovarian cancer cells, endometrial cancer cells, adenocarcinoma of the colon, pancreatic carcinoma cells or small cell lung cancer cells, or wherein the tumor cells are melanoma cells.

21. The method of claim 13, wherein the chemotherapeutic drug is a DNA damaging agent, preferably selected from the group consisting of actinomycin-D, mitomycin C, cisplatin, doxorubicin, etoposide, gemcitabine, verapamil, podophyllotoxin, 5-FU, taxans, preferably paclitaxel, carboplatin, cyclophosphamide, vinorelbine, oxaliplatin, capecitabine, doxorubicin, and ifosfamide.

22. The method of claim 13, wherein the radiotherapy is selected from the group consisting of X-ray radiation, UV-radiation, γ-irradiation and microwaves.

23. The method of claim 13, wherein the L1 interfering molecule is an anti-L1 antibody or an antibody fragment thereof.

24. The method of claim 1, wherein the L1 interfering molecule promotes apoptosis in the tumor cells.
